(19) United States
(12) Patent Application Publication
(10) Pub. No.: US 2010/0226881 A1
(43) Pub. Date: Sep. 9, 2010

(54) PYRAZOLO[1,5-a]PYRIDINES AND THEIR USE IN CANCER THERAPY

(75) Inventors: Jackie Diane Kendall, Auckland (NZ); Andrew James Marshall, Drury (NZ)

Correspondence Address: EDWARDS ANGELL PALMER & DODGE LLP, P.O. BOX 55874, BOSTON, MA, 02205, US

(73) Assignee: Auckland Uniservices Limited, Auckland (NZ)

(21) Appl. No.: 12/452,536

(22) PCT Filed: Jul. 11, 2008
(86) PCT No.: PCT/NZ2008/000164

§ 371 Date: May 24, 2010

Related U.S. Application Data
(60) Provisional application No. 60/949,203, filed on Jul. 11, 2007.

(51) Int. Cl. A61K 31/437; C07D 471/04; A61K 33/24; A61K 31/4985; A61K 31/7034; A61K 38/21; A61P 35/00

(52) U.S. Cl. 424/85.5; 546/121; 514/300; 424/649; 514/249; 514/34; 424/85.6; 424/85.7

(57) ABSTRACT

Pyrazolo[1,5-a]pyridines are described, including methods for their preparation, and their use as agents or drugs for cancer therapy, both alone or in combination with radiation and/or other anticancer drugs.

### TECHNICAL FIELD

The present invention relates to pyrazolo[1,5-a]pyridines, to their preparation, to their use as agents or drugs for cancer therapy, both alone or in combination with radiation and/or other anticancer drugs.

### BACKGROUND TO THE INVENTION

Phosphoinositide-3-kinases (PI3Ks) are a group of lipid kinases which phosphorylate the 3-hydroxyl of phosphoinositides. They are split into three classes (Class I, II and III) and play an important role in cellular signalling [Stephens et al., Curr. Opin. Pharmacol. 2005, 5, 357]. The Class I enzymes are further split into Class Ia and Ib based on their mechanism of activation; the Class Ia PI3Ks are heterodimeric structures consisting of a catalytic subunit (p110α, p110β or p110δ) in complex with a regulatory p85 subunit, while the class-IB PI3K (p110γ) is structurally similar but lacks a regulatory subunit linking and instead is activated by βγ subunits of heterotrimeric G-proteins [Walker et al., Mol. Cell., 2000, 6, 909].

PI3Ks play a variety of roles in normal tissue physiology [Foukas & Shepherd, Biochem. Soc. Trans., 2004, 32, 330; Shepherd, Acta Physiol. Scand,. 2005, 183, 3], with p110α having a specific role in cancer growth, p110β in thrombus formation mediated by integrin α_{II}β₃ [Jackson et al., Nat. Med., 2005, 11, 507], and p110γ in inflammation, rheumatoid arthritis [Camps et al., Nat. Med., 2005, 11, 936] and other chronic inflammation states [Barber et al., Nat. Med., 2005, 11, 933]. The PI3K enzymes produce phosphoinositide 3,4,5-triphosphate (PIP3) from the corresponding diphosphate (PIP2), thus recruiting AKT (protein kinase B) through its PH domain, to the plasma membrane. Once bound, AKT is phosphorylated and activated by other membrane bound kinases, and is central to a cascade of events that lead to inhibition of apoptosis [Berrie, Exp. Opin. Invest. Drugs, 2001, 10, 1085].

The p110α isoform is selectively amplified and activated, in a number of cancer types [Stephens et al., Curr. Opin. Pharmacol., 2005, 5, 357; Stauffer et al., Curr. Med. Chem—Anti-Cancer Agents, 2005, 5, 449], and in addition there is a high frequency of non-random mutations in specific sites (primarily in the C2 domain and or the activation loop) of the kinase in several human cancer cell lines, including colon, brain, breast and stomach. This results in a constitutively active enzyme [Ikenoue et al., Cancer Res., 2005, 65, 4562; Kang et al., Proc. Natl. Acad. Sci. USA, 2005, 102, 802], making p110α one of the most highly mutated oncogenes found in human tumours.

While PI3K isoenzymes play important roles in many cellular processes, published experimental studies in mice with human tumour xenografts show that the pan-PI3K inhibitor LY294002 is well-tolerated, reduces signalling through the PI3K pathway, and causes reduction of tumour volume, and is more active in cell lines over-expressing mutant forms of p110α than parental control cells [Semba et al., Clin. Cancer Res., 2002, 8, 1957; Hu et al., Cancer Res., 2002, 62, 1087].

Thus PI3K, and especially the p110α isoenzyme, is an interesting target for drug intervention, and several classes of compounds have been identified as inhibitors, as exemplified by LY240002 (non-selective) [Walker et al., Mol. Cell., 2000, 6, 909], PI103 (slightly α-selective) [Knight et al., Cell, 2006, 125, 733], ZSTK474 (non-selective) [Yaguchi et al., J. Natl. Cancer Inst., 2006, 98, 545], TGX221 (β-selective) [Jackson et al., Nat. Med., 2005, 11, 507], oxazines (γ-selective) [Lanni et al., Bioorg. Med. Chem. Lett., 2007, 17, 756], IC87144 (δ-selective) [Sadhu et al., PCT Int Appl. WO 0181346, November 2001], AS605240 (γ-selective [Camps et al., Nat. Med., 2005, 11, 936] and the imidazo[1,2-a]pyridines (α-selective) [Hayakawa et al., U.S. Pat. No. 6,403,588, 2002]. A systematic study of analogues of the latter chemotype indicated tight structure-activity relationships for the two-ring moiety, with no compounds superior to the imidazo[1,2-a]pyridine [Kendall et al., Bioorg. Med. Chem., 2007, 15, 7677].

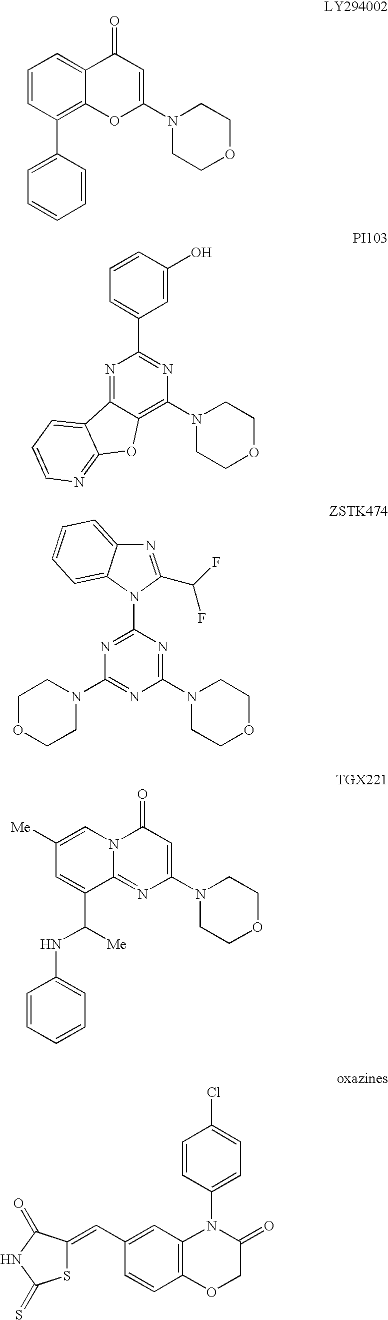

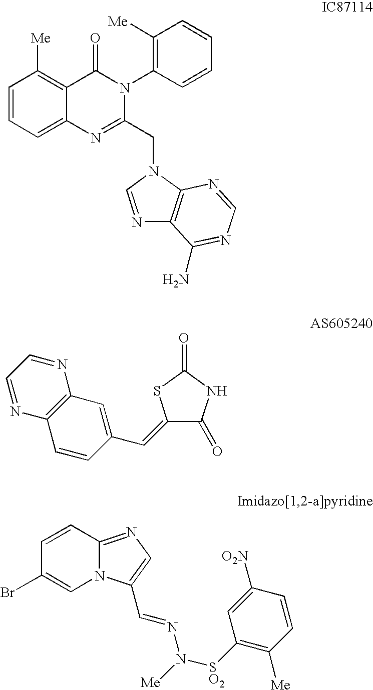

It is an object of the present invention to provide a class of pyrazolo[1,5-a]pyridines as anticancer drugs, or to at least provide the public with a useful alternative.

### SUMMARY OF THE INVENTION

In a first aspect there is provided a compound of Formula (I),

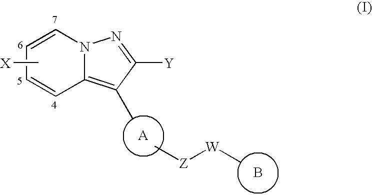

wherein;
X may represent up to two of R, F, Cl, Br, I, OR, OCOR, CONR₂, CO₂R, SO₂R, SO₂NR₂, CN, CF₃, OCF₃, NO₂, NR₂, NHCOR or optionally substituted aryl, placed at any of the available positions 4-, 5-, 6-, 7;
R may be H or C1-C6 saturated or unsaturated alkyl optionally substituted with halogen, OH, OR¹, NHR¹, NR¹₂, or optionally substituted aryl or heteroaryl, or in the case where R forms part of NR₂ this may form an optionally substituted 4-7 membered saturated ring optionally containing one additional heteroatom from the group O, S, NR²;
R¹ is H, C1-C6 saturated or unsaturated alkyl, or optionally substituted aryl or heteroaryl, or in the case when R¹ forms part of NR¹₂ this may form an optionally substituted 4-7 membered saturated ring optionally containing one additional heteroatom from the group O, S, NR²;
R² is H or C1-C6 saturated or unsaturated alkyl;

### Y may be H or CH₃;

A represents [symbol] -CH═N—N(R)- [symbol] (where [symbol] is linked to the 3-position of the pyrazole ring of formula I and [symbol] is linked to Z), or any 5-membered heterocylic ring containing up to three of the atoms S, O or N in the ring, and optionally substituted with R, as defined above.
Z represents SOₓ (where x=0-2), CH₂ or CO;
W is absent or (CH₂)_{y} where y=1, 2 or 3;
B is phenyl, naphthyl, or 5- or 6-membered heterocycle or benzoheterocycle, where the heterocylic ring contains up to two of the atoms S, O or N, optionally substituted at any available position with T, which is up to two of F, Cl, Br, I, R, OR, CONR₂, CO₂R, SO₂R, SO₂NHR, CN, CF₃, OCF₃, NO₂, NR₂, NHCOR, where R is defined as above;
or a physiologically acceptable salt or phosphate prodrug or carboxylic acid or amino acid ester prodrug thereof.

In one embodiment, X is substituted at the 5-position with R, halogen, OR, OCOR, CONR₂, CO₂R, SO₂R, SO₂NR₂, CN, CF₃, OCF₃, NO₂, NR₂, NHCOR or optionally substituted aryl, where R is defined as above.

In another embodiment, Z is SO₂ and W is absent.

In a further embodiment, B is phenyl, optionally substituted at any position with T.

In a further embodiment, A is selected from formulae IIa-IIe, where [symbol] is linked to the 3-position of the pyrazole ring of formula (I) and [symbol] is linked to Z, where R is defined as above:

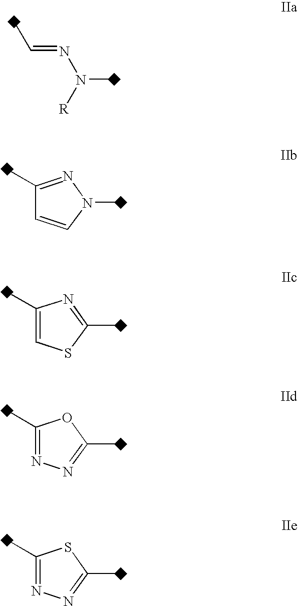

In one embodiment, the compound of formula I as defined above is selected from:
N,2-Dimethyl-5-nitro-N′-(pyrazolo[1,5-a]pyridin-3-ylmethylene)benzenesulfonohydrazide
N,2-Dimethyl-N′-((5-methylpyrazolo[1,5-a]pyridin-3-yl)methylene)-5-nitrobenzene-sulfonohydrazide
N,2-Dimethyl-5-nitro-N′-((5-(trifluoromethyl)pyrazolo[1,5-a]pyridin-3-yl)methylene)-benzenesulfonohydrazide
N′-((2,5-Dimethylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N,2-dimethyl-5-nitrobenzene-sulfonohydrazide
Methyl 3-((2-methyl-2-(2-methyl-5-nitrophenylsulfonyl)hydrazono)methyl)pyrazolo[1,5-a]pyridine-5-carboxylate
3-((2-Methyl-2-(2-methyl-5-nitrophenylsulfonyl)hydrazono)methyl)pyrazolo[1,5-a]pyridine-5-carboxamide
N′-((5-(2-Hydroxyethyl)pyrazolo[1,5-a]pyridin-3-yl)methylene)-N,2-dimethyl-5-nitro-benzenesulfonohydrazide
N′-((5-Methoxypyrazolo[1,5-a]pyridin-3-yl)methylene)-N,2-dimethyl-5-nitrobenzene-sulfonohydrazide
3-((2-Methyl-2-(2-methyl-5-nitrophenylsulfonyl)hydrazono)methyl)pyrazolo[1,5-a]pyridin-5-ylacetate
N′-((5-Hydroxypyrazolo[1,5-a]pyridin-3-yl)methylene)-N,2-dimethyl-5-nitrobenzene-sulfonohydrazide
N′-((5-Aminopyrazolo[1,5-a]pyridin-3-yl)methylene)-N,2-dimethyl-5-nitrobenzene-sulfonohydrazide
N′-((5-Chloropyrazolo[1,5-a]pyridin-3-yl)methylene)-N,2-dimethyl-5-nitrobenzene-sulfonohydrazide
N′-((5-Bromopyrazolo[1,5-a]pyridin-3-yl)methylene)-N,2-dimethyl-5-nitrobenzene-sulfonohydrazide
N′-((5-Iodopyrazolo[1,5-a]pyridin-3-yl)methylene)-N,2-dimethyl-5-nitrobenzene-sulfonohydrazide
N,2-Dimethyl-5-nitro-N′-((5-vinylpyrazolo[1,5-a]pyridin-3-yl)methylene)benzene-sulfonohydrazide
N′-((5-Cyclopropylpyrazolo[1,5-a]pyridin-3-yl)methylene)-N,2-dimethyl-5-nitro-benzenesulfonohydrazide
N′-((5-Ethynylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N-methyl-3-nitrobenzenesulfonohydrazide
3-Cyano-N′-((5-cyanopyrazolo[1,5-a]pyridin-3-yl)methylene)-N-methylbenzene-sulfonohydrazide
5-Cyano-N′-((5-cyanopyrazolo[1,5-a]pyridin-3-yl)methylene)-N,2-dimethylbenzene-sulfonohydrazide
N′-((5-Cyanopyrazolo[1,5-a]pyridin-3-yl)methylene)-N-methyl-3-(trifluoromethyl)-benzenesulfonohydrazide
N′-((5-Cyanopyrazolo[1,5-a]pyridin-3-yl)methylene)-N,2-dimethyl-5-(trifluoromethyl)-benzenesulfonohydrazide
N′-((5-Cyanopyrazolo[1,5-a]pyridin-3-yl)methylene)-N,2-dimethylbenzenesulfonohydrazide
N′-((5-Cyanopyrazolo[1,5-a]pyridin-3-yl)methylene)-N-methyl-4-nitrobenzenesulfonohydrazide
N′-((5-Cyanopyrazolo[1,5-a]pyridin-3-yl)methylene)-N,2-dimethyl-4-nitrobenzenesulfonohydrazide
N′-((5-Cyanopyrazolo[1,5-a]pyridin-3-yl)methylene)-5-fluoro-N,2-dimethylbenzene-sulfonohydrazide
5-Bromo-N′-((5-cyanopyrazolo[1,5-a]pyridin-3-yl)methylene)-N,2-dimethyl-benzene-sulfonohydrazide
3-Bromo-N′-((5-cyanopyrazolo[1,5-a]pyridin-3-yl)methylene)-N-methylbenzene-sulfonohydrazide
Methyl 3-(2-((5-cyanopyrazolo[1,5-a]pyridin-3-yl)methylene)-1-methylhydrazinylsulfonyl)-4-methylbenzoate
N′-((5-Cyanopyrazolo[1,5-a]pyridin-3-yl)methylene)-N,2-dimethyl-5-(methylsulfonyl)-benzenesulfonohydrazide
N′-((5-Cyanopyrazolo[1,5-a]pyridin-3-yl)methylene)-2-ethyl-N-methyl-5-nitrobenzene-sulfonohydrazide
N′-((5-Cyanopyrazolo[1,5-a]pyridin-3-yl)methylene)-2-isopropyl-N-methyl-5-nitro-benzenesulfonohydrazide
2-Chloro-N′-((5-cyanopyrazolo[1,5-a]pyridin-3-yl)methylene)-N-methyl-5-nitro-benzenesulfonohydrazide
N′-((5-Cyanopyrazolo[1,5-a]pyridin-3-yl)methylene)-2-methoxy-N-methyl-5-nitro-benzenesulfonohydrazide
N′-((5-Cyanopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-5-cyano-N,2-dimethylbenzene-sulfonohydrazide
N′-((5-Cyanopyrazolo[1,5-a]pyridin-3-yl)methylene)-N-(2-hydroxyethyl)-2-methyl-5-nitrobenzenesulfonohydrazide
N′-((5-Bromopyrazolo[1,5-a]pyridin-3-yl)methylene)-N-(2-hydroxyethyl)-2-methyl-5-nitrobenzenesulfonohydrazide
5-Cyano-N′-((5-cyanopyrazolo[1,5-a]pyridin-3-yl)methylene)-N-(2-hydroxyethyl)-2-methylbenzenesulfonohydrazide
N-(2-Hydroxyethyl)-2-methyl-5-nitro-N′-(pyrazolo[1,5-a]pyridin-3-ylmethylene)-benzenesulfonohydrazide
N′-((5-Cyanopyrazolo[1,5-a]pyridin-3-yl)methylene)-2-methyl-5-nitrobenzenesulfonohydrazide
N-Benzyl-N′-((5-cyanopyrazolo[1,5-a]pyridin-3-yl)methylene)-2-methyl-5-nitro-benzenesulfonohydrazide
N′-((5-Cyanopyrazolo[1,5-a]pyridin-3-yl)methylene)-N-ethyl-2-methyl-5-nitrobenzene-sulfonohydrazide
N′-((5-Cyanopyrazolo[1,5-a]pyridin-3-yl)methylene)-N-(2-(diethylamino)ethyl)-2-methyl-5-nitrobenzenesulfonohydrazide
N′-((5-Cyanopyrazolo[1,5-a]pyridin-3-yl)methylene)-N-(2-(dimethylamino)ethyl)-2-methyl-5-nitrobenzenesulfonohydrazide
N′-((5-Cyanopyrazolo[1,5-a]pyridin-3-yl)methylene)-2-methyl-N-(2-morpholinoethyl)-5-nitrobenzenesulfonohydrazide
N′-((5-Cyanopyrazolo[1,5-a]pyridin-3-yl)methylene)-2-methyl-5-nitro-N-(2-(piperidin-1-yl)ethyl)benzenesulfonohydrazide
N′-((5-Cyanopyrazolo[1,5-a]pyridin-3-yl)methylene)-2-methyl-5-nitro-N-(2-(pyrrolidin-1-yl)ethyl)benzenesulfonohydrazide
N′-((5-Bromopyrazolo[1,5-a]pyridin-3-yl)methylene)-2-methyl-5-nitrobenzenesulfonohydrazide
N′-((5-Bromopyrazolo[1,5-a]pyridin-3-yl)methylene)-2-methyl-5-nitro-N-(2-(piperidin-1-yl)ethyl)benzenesulfonohydrazide hydrochloride
N′-((5-Bromopyrazolo[1,5-a]pyridin-3-yl)methylene)-2-methyl-5-nitro-N-(3-(piperidin-1-yl)propyl)benzenesulfonohydrazide hydrochloride
N′-((5-Bromopyrazolo[1,5-a]pyridin-3-yl)methylene)-2-methyl-N-(3-morpholinopropyl)-5-nitrobenzenesulfonohydrazide hydrochloride
2-Methyl-N′-((5-methylpyrazolo[1,5-a]pyridin-3-yl)methylene)-5-nitrobenzenesulfonohydrazide
N′-((5-Cyanopyrazolo[1,5-a]pyridin-3-yl)methylene)-2-fluoro-N-methyl-5-nitro-benzenesulfonohydrazide
N′-((5-Cyanopyrazolo[1,5-a]pyridin-3-yl)methylene)-2-((2-(dimethylamino)ethyl)-(methyl)amino)-N-methyl-5-nitrobenzenesulfonohydrazide
N′-((5-Cyanopyrazolo[1,5-a]pyridin-3-yl)methylene)-2-(2-(dimethylamino)ethylamino)-N-methyl-5-nitrobenzenesulfonohydrazide hydrochloride
N′-((5-Cyanopyrazolo[1,5-a]pyridin-3-yl)methylene)-N-methyl-2-(2-morpholinoethylamino)-5-nitrobenzenesulfonohydrazide hydrochloride
N′-((5-Cyanopyrazolo[1,5-a]pyridin-3-yl)methylene)-N-methyl-2-(methyl(2-morpholinoethyl)amino)-5-nitrobenzenesulfonohydrazide hydrochloride
N′-((5-Cyanopyrazolo[1,5-a]pyridin-3-yl)methylene)-N-methyl-2-(methyl(2-(piperidin-1-yl)ethyl)amino)-5-nitrobenzenesulfonohydrazide hydrochloride
N′-((5-Cyanopyrazolo[1,5-a]pyridin-3-yl)methylene)-N-methyl-2-(3-morpholinopropyl-amino)-5-nitrobenzenesulfonohydrazide hydrochloride
N′-((5-Cyanopyrazolo[1,5-a]pyridin-3-yl)methylene)-N-methyl-2-(methyl(2-(methyl-amino)ethyl)amino)-5-nitrobenzenesulfonohydrazide hydrochloride
2-(2-(1H-Imidazol-4-yl)ethylamino)-N′-((5-cyanopyrazolo[1,5-a]pyridin-3-yl)methylene)-N-methyl-5-nitrobenzenesulfonohydrazide hydrochloride
N′-((5-Cyanopyrazolo[1,5-a]pyridin-3-yl)methylene)-N-methyl-5-nitro-2-(pyridin-2-yl-methylamino)benzenesulfonohydrazide hydrochloride
N′-((5-Cyanopyrazolo[1,5-a]pyridin-3-yl)methylene)-N-methyl-5-nitro-2-(pyridin-3-yl-methylamino)benzenesulfonohydrazide hydrochloride
N′-((5-Cyanopyrazolo[1,5-a]pyridin-3-yl)methylene)-N-methyl-5-nitro-2-(pyridin-4-yl-methylamino)benzenesulfonohydrazide hydrochloride
N′-((5-Cyanopyrazolo[1,5-a]pyridin-3-yl)methylene)-N-methyl-2-(methyl(pyridin-3-yl-methyl)amino)-5-nitrobenzenesulfonohydrazide hydrochloride
N′-((5-Cyanopyrazolo[1,5-a]pyridin-3-yl)methylene)-2-(2-(dimethylamino)ethoxy)-N-methyl-5-nitrobenzenesulfonohydrazide hydrochloride
N′-((5-Cyanopyrazolo[1,5-a]pyridin-3-yl)methylene)-N-methyl-2-(2-morpholinoethoxy)-5-nitrobenzenesulfonohydrazide hydrochloride
N′-((5-Cyanopyrazolo[1,5-a]pyridin-3-yl)methylene)-N-methyl-5-nitro-2-(2-(pyrrolidin-1-yl)ethoxy)benzenesulfonohydrazide hydrochloride
N′-((5-Cyanopyrazolo[1,5-a]pyridin-3-yl)methylene)-N-methyl-5-nitro-2-(pyridin-2-yl-methoxy)benzenesulfonohydrazide hydrochloride
N′-((5-Bromopyrazolo[1,5-a]pyridin-3-yl)methylene)-2-fluoro-N-methyl-5-nitro-benzenesulfonohydrazide
N′-((5-Bromopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-N-methyl-2-(2-morpholinoethoxy)-5-nitrobenzenesulfonohydrazide hydrochloride
5-Cyano-N′-((5-cyanopyrazolo[1,5-a]pyridin-3-yl)methylene)-2-fluoro-N-methyl-benzenesulfonohydrazide
5-Cyano-N′-((5-cyanopyrazolo[1,5-a]pyridin-3-yl)methylene)-2-(dimethylamino)-N-methylbenzenesulfonohydrazide
5-Cyano-N′-((5-cyanopyrazolo[1,5-a]pyridin-3-yl)methylene)-2-((2-(dimethylamino)-ethyl)(methyl)amino)-N-methylbenzenesulfonohydrazide hydrochloride
5-Cyano-N′-((5-cyanopyrazolo[1,5-a]pyridin-3-yl)methylene)-N-methyl-2-(2-morpholinoethylamino)benzenesulfonohydrazide hydrochloride
5-Cyano-N′-((5-cyanopyrazolo[1,5-a]pyridin-3-yl)methylene)-N-methyl-2-(2-morpholinoethoxy)benzenesulfonohydrazide hydrochloride
2-Chloro-N′-((5-cyanopyrazolo[1,5-a]pyridin-3-yl)methylene)-N-methyl-5-nitropyridine-3-sulfonohydrazide
N′-((5-Cyanopyrazolo[1,5-a]pyridin-3-yl)methylene)-2-((2-(dimethylamino)ethyl)-(methyl)amino)-N-methyl-5-nitropyridine-3-sulfonohydrazide hydrochloride
N′-((5-Cyanopyrazolo[1,5-a]pyridin-3-yl)methylene)-N-methyl-3-nitrobenzohydrazide
N′-((5-Cyanopyrazolo[1,5-a]pyridin-3-yl)methylene)-N,2-dimethyl-5-nitrobenzohydrazide
3-((2-Methyl-2-(2-methyl-5-nitrobenzyl)hydrazono)methyl)pyrazolo[1,5-a]pyridine-5-carbonitrile
3-(1-(2-Methyl-5-nitrophenylsulfonyl)-1H-pyrazol-3-yl)pyrazolo[1,5-a]pyridine
3-(1-(3-Nitrophenylsulfonyl)-1H-pyrazol-3-yl)pyrazolo[1,5-a]pyridine
3-(3-(Pyrazolo[1,5-a]pyridin-3-yl)-1H-pyrazol-1-ylsulfonyl)benzonitrile
5-Bromo-3-(1-(2-methyl-5-nitrophenylsulfonyl)-1H-pyrazol-3-yl)pyrazolo[1,5-a]pyridine
4-(5-Bromopyrazolo[1,5-a]pyridin-3-yl)-2-(3-nitrophenylsulfinyl)thiazole
4-(5-Bromopyrazolo[1,5-a]pyridin-3-yl)-2-(3-nitrophenylsulfonyl)thiazole
2-(5-Bromopyrazolo[1,5-a]pyridin-3-yl)-5-(2-methyl-5-nitrophenylthio)-1,3,4-oxadiazole
2-(5-Bromopyrazolo[1,5-a]pyridin-3-yl)-5-(2-methyl-5-nitrophenylsulfinyl)-1,3,4-oxadiazole
2-(5-Bromo-2-methylphenylsulfonyl)-5-(5-bromopyrazolo[1,5-a]pyridin-3-yl)-1,3,4-thiadiazole

It is appreciated that compounds of Formula I may occur in different geometric and enantiomeric forms, and that both pure forms and mixtures of these separate isomers are included, and any physiologically acceptable salts or phosphate or carboxylic acid or amino acid ester prodrugs thereof.

In a second aspect there is provided a method of cancer prevention or therapy for treating cancers including the step of administering a compound of Formula I as defined above.

In one embodiment the method further includes administering one or more chemotherapeutic agents and/or therapies. In one embodiment the agents and/or therapies are selected from:
Alkylation agents (eg cisplatin, carboplatin)
Antimetabolites (eg methotrexate, 5-FU)
Antitumour antibiotics (eg adriamymycin, bleomycin)
Antitumour vegetable alkaloids (eg taxol, etoposide)
Antitumor hormones (eg dexamethasone, tamoxifen)
Antitumour immunological agents (eg interferon α, β, γ)
Radiation therapy
Surgery

In one embodiment the method further includes the step of administering one or more chemotherapeutic agents to the subject before, during or after the administration of the compound of Formula I as defined above in the second aspect of the invention to the subject.

While these compounds of Formula I will typically be used in cancer prevention or cancer therapy of human subjects, they can be used to target cancer cells in other warm blooded animal subjects such as other primates, farm animals such as cattle, and sports animals and pets such as horses, dogs, and cats. It will be appreciated that reference to cancer prevention or cancer therapy is not intended to be a reference to a cure for cancer or to absolute prevention. The reference is intended to include reference to a reduction in the likelihood of contraction of cancer or a mitigation of development, or like outcome.

In a third aspect there is provided a pharmaceutical composition including a compound of Formula I as defined above in the first aspect, and a pharmaceutically acceptable excipient, adjuvant, carrier, buffer or stabiliser.

The pharmaceutically acceptable excipient, adjuvant, carrier, buffer or stabiliser should be non-toxic and should not interfere with the efficacy of the active ingredient. The precise nature of the carrier or other material will depend on the route of administration, which may be oral, or by injection, such as cutaneous, subcutaneous, or intravenous injection. The composition may therefore be in a tablet, capsule, powder, or liquid form. In one embodiment the composition will is suitable for oral or parenteral administration.

In one embodiment, the pharmaceutical compositions further include one or more chemotherapeutic agents as defined in the second aspect.

In a fourth aspect there is provided the use of a compound of Formula I in the manufacture of a medicament for the treatment of cancer. In one embodiment, the medicament is in tablet, capsule, powder or liquid form. In one embodiment, the composition is suitable for oral or parenteral administration.

In a fifth aspect there is provided a method of making a compound of Formula I as defined above, the method including the step of modifying a pyrazolo[1,5-a]pyridine-3-carbonyl compound of Formula III

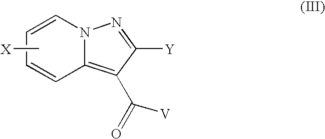

wherein variables X and Y are as defined above for Formula I and V is H, CH₃ or alkoxy.

In one embodiment, where V is alkoxy is it ethoxy (OEt).

In one embodiment, compounds according to Formula I can be prepared according to any one of routes (I) to (IX) as described later herein in Scheme 1, in which A, R, T, X, Y, Z are as defined for Formula 1.

In one embodiment, when X and Y are as defined above for Formula I and V is CH₃, the method of preparing compound of Formula I from compound of Formula III involves one of the following:
(i) reaction with DMFdma then cyclisation with hydrazine, followed by sulfonylation as illustrated in Scheme 27 below; or
(ii) bromination then cyclisation with dithiocarbamate, coupling with a boronic acid followed by oxidation as illustrated in Schemes 28 and 29 below.

In one embodiment, when X and Y are as defined above for Formula I and V is OEt, the method of preparing compound of Formula I from compound of Formula III involves one of the following:
(i) reaction with hydrazine then cyclisation with CS₂, coupling with a boronic acid followed by oxidation as illustrated in Scheme 30 below; or
(ii) basic hydrolysis, then reaction with thiosemicarbazide, cyclisation with H₂SO₄, followed by diazotisation and chlorination, then substitution with a sulfinate salt as illustrated in Schemes 3 and 31 below.

In one embodiment, the method of preparing compound of Formula I from compound of Formula III involves reaction with DMFdma then cyclisation with hydrazine and proceeds via an intermediate of Formula IV

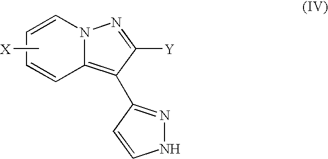

wherein variables X and Y are as defined above for Formula I.

Alternatively, when X and Y are as defined above for Formula I and V is H, the method for preparing compound of Formula I from compound of Formula III involves one of the following:
(i) condensation with a hydrazine followed by sulfonylation as illustrated in Schemes 15 and 21 below;
(ii) condensation with a sulfonohydrazide followed by alkylation as illustrated in Schemes 16 and 22 below;
(iii) reaction with methylhydrazine sulphate followed by acylation as illustrated in Scheme 24 below;
(iv) condensation with a hydrazine as illustrated in Scheme 25 below; or,
(v) condensation with methylhydrazine sulphate followed by sulfonylation and then nucleophilic substitution as illustrated in Scheme 23 below.

In one embodiment, the method of preparing a compound of Formula I from a compound of Formula III include a route via the:
(a) preparation of compounds of Formula V and VI substantially in accordance with Scheme 28 (defined later herein); or
(b) preparation of compounds of Formula VII and VIII substantially in accordance with Scheme 30 (defined later herein); or
(c) preparation of compounds of Formula IX and X substantially in accordance with Scheme 31 (defined later herein); wherein

Formula V to X (in which X and Y are as defined for Formula I) are:

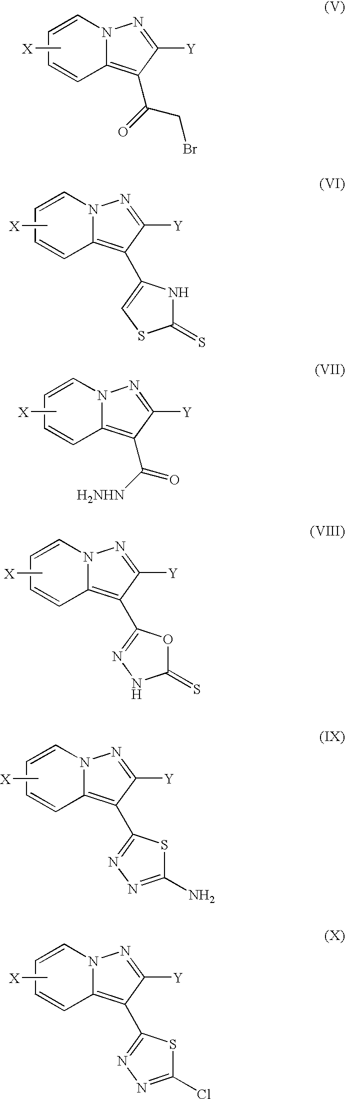

In a sixth aspect there is provided a compound of Formula I obtained by the methods of the fifth aspect.

In one embodiment the compound of Formula I obtained by a method of the fifth aspect is selected from one or more of the following:
N,2-Dimethyl-5-nitro-N′-(pyrazolo[1,5-a]pyridin-3-ylmethylene)benzenesulfonohydrazide
N,2-Dimethyl-N′-((5-methylpyrazolo[1,5-a]pyridin-3-yl)methylene)-5-nitrobenzene-sulfonohydrazide
N,2-Dimethyl-5-nitro-N′-((5-(trifluoromethyl)pyrazolo[1,5-a]pyridin-3-yl)methylene)-benzenesulfonohydrazide
N′-((2,5-Dimethylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N,2-dimethyl-5-nitrobenzene-sulfonohydrazide
Methyl 3-((2-methyl-2-(2-methyl-5-nitrophenylsulfonyl)hydrazono)methyl)pyrazolo[1,5-a]pyridine-5-carboxylate
3-((2-Methyl-2-(2-methyl-5-nitrophenylsulfonyl)hydrazono)methyl)pyrazolo[1,5-a]pyridine-5-carboxamide
N′-((5-(2-Hydroxyethyl)pyrazolo[1,5-a]pyridin-3-yl)methylene)-N,2-dimethyl-5-nitro-benzenesulfonohydrazide
N′-((5-Methoxypyrazolo[1,5-a]pyridin-3-yl)methylene)-N,2-dimethyl-5-nitrobenzene-sulfonohydrazide
3-((2-Methyl-2-(2-methyl-5-nitrophenylsulfonyl)hydrazono)methyl)pyrazolo[1,5-a]pyridin-5-yl acetate
N′-((5-Hydroxypyrazolo[1,5-a]pyridin-3-yl)methylene)-N,2-dimethyl-5-nitrobenzene-sulfonohydrazide
N′-((5-Aminopyrazolo[1,5-a]pyridin-3-yl)methylene)-N,2-dimethyl-5-nitrobenzene-sulfonohydrazide
N′-((5-Chloropyrazolo[1,5-a]pyridin-3-yl)methylene)-N,2-dimethyl-5-nitrobenzene-sulfonohydrazide
N′-((5-Bromopyrazolo[1,5-a]pyridin-3-yl)methylene)-N,2-dimethyl-5-nitrobenzene-sulfonohydrazide
N′-((5-Iodopyrazolo[1,5-a]pyridin-3-yl)methylene)-N,2-dimethyl-5-nitrobenzene-sulfonohydrazide
N,2-Dimethyl-5-nitro-N′-((5-vinylpyrazolo[1,5-a]pyridin-3-yl)methylene)benzene-sulfonohydrazide
N′-((5-Cyclopropylpyrazolo[1,5-a]pyridin-3-yl)methylene)-N,2-dimethyl-5-nitro-benzenesulfonohydrazide
N′-((5-Ethynylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N-methyl-3-nitrobenzenesulfonohydrazide
3-Cyano-N′-((5-cyanopyrazolo[1,5-a]pyridin-3-yl)methylene)-N-methylbenzene-sulfonohydrazide
5-Cyano-N′-((5-cyanopyrazolo[1,5-a]pyridin-3-yl)methylene)-N,2-dimethylbenzene-sulfonohydrazide
N′-((5-Cyanopyrazolo[1,5-a]pyridin-3-yl)methylene)-N-methyl-3-(trifluoromethyl)-benzenesulfonohydrazide
N′-((5-Cyanopyrazolo[1,5-a]pyridin-3-yl)methylene)-N,2-dimethyl-5-(trifluoromethyl)-benzenesulfonohydrazide
N′-((5-Cyanopyrazolo[1,5-a]pyridin-3-yl)methylene)-N,2-dimethylbenzenesulfonohydrazide
N′-((5-Cyanopyrazolo[1,5-a]pyridin-3-yl)methylene)-N-methyl-4-nitrobenzenesulfonohydrazide
N′-((5-Cyanopyrazolo[1,5-a]pyridin-3-yl)methylene)-N,2-dimethyl-4-nitrobenzene-sulfonohydrazide
N′-((5-Cyanopyrazolo[1,5-a]pyridin-3-yl)methylene)-5-fluoro-N,2-dimethylbenzene-sulfonohydrazide
5-Bromo-N′-((5-cyanopyrazolo[1,5-a]pyridin-3-yl)methylene)-N,2-dimethyl-benzene-sulfonohydrazide
3-Bromo-N′-((5-cyanopyrazolo[1,5-a]pyridin-3-yl)methylene)-N-methylbenzene-sulfonohydrazide
Methyl 3-(2-((5-cyanopyrazolo[1,5-a]pyridin-3-yl)methylene)-1-methylhydrazinylsulfonyl)-4-methylbenzoate
N′-((5-Cyanopyrazolo[1,5-a]pyridin-3-yl)methylene)-N,2-dimethyl-5-(methylsulfonyl)-benzenesulfonohydrazide
N′-((5-Cyanopyrazolo[1,5-a]pyridin-3-yl)methylene)-2-ethyl-N-methyl-5-nitrobenzene-sulfonohydrazide
N′-((5-Cyanopyrazolo[1,5-a]pyridin-3-yl)methylene)-2-isopropyl-N-methyl-5-nitro-benzenesulfonohydrazide
2-Chloro-N′-((5-cyanopyrazolo[1,5-a]pyridin-3-yl)methylene)-N-methyl-5-nitro-benzenesulfonohydrazide
N′-((5-Cyanopyrazolo[1,5-a]pyridin-3-yl)methylene)-2-methoxy-N-methyl-5-nitro-benzenesulfonohydrazide
N′-((5-Cyanopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-5-cyano-N,2-dimethylbenzene-sulfonohydrazide
N′-((5-Cyanopyrazolo[1,5-a]pyridin-3-yl)methylene)-N-(2-hydroxyethyl)-2-methyl-5-nitrobenzenesulfonohydrazide
N′-((5-Bromopyrazolo[1,5-a]pyridin-3-yl)methylene)-N-(2-hydroxyethyl)-2-methyl-5-nitrobenzenesulfonohydrazide
5-Cyano-N′-((5-cyanopyrazolo[1,5-a]pyridin-3-yl)methylene)-N-(2-hydroxyethyl)-2-methylbenzenesulfonohydrazide
N-(2-Hydroxyethyl)-2-methyl-5-nitro-N′-(pyrazolo[1,5-a]pyridin-3-ylmethylene)-benzenesulfonohydrazide
N′-((5-Cyanopyrazolo[1,5-a]pyridin-3-yl)methylene)-2-methyl-5-nitrobenzenesulfonohydrazide
N-Benzyl-N′-((5-cyanopyrazolo[1,5-a]pyridin-3-yl)methylene)-2-methyl-5-nitro-benzenesulfonohydrazide
N′-((5-Cyanopyrazolo[1,5-a]pyridin-3-yl)methylene)-N-ethyl-2-methyl-5-nitrobenzene-sulfonohydrazide
N′-((5-Cyanopyrazolo[1,5-a]pyridin-3-yl)methylene)-N-(2-(diethylamino)ethyl)-2-methyl-5-nitrobenzenesulfonohydrazide
N′-((5-Cyanopyrazolo[1,5-a]pyridin-3-yl)methylene)-N-(2-(dimethylamino)ethyl)-2-methyl-5-nitrobenzenesulfonohydrazide
N′-((5-Cyanopyrazolo[1,5-a]pyridin-3-yl)methylene)-2-methyl-N-(2-morpholinoethyl)-5-nitrobenzenesulfonohydrazide
N′-((5-Cyanopyrazolo[1,5-a]pyridin-3-yl)methylene)-2-methyl-5-nitro-N-(2-(piperidin-1-yl)ethyl)benzenesulfonohydrazide
N′-((5-Cyanopyrazolo[1,5-a]pyridin-3-yl)methylene)-2-methyl-5-nitro-N-(2-(pyrrolidin-1-yl)ethyl)benzenesulfonohydrazide
N′-((5-Bromopyrazolo[1,5-a]pyridin-3-yl)methylene)-2-methyl-5-nitrobenzenesulfonohydrazide
N′-((5-Bromopyrazolo[1,5-a]pyridin-3-yl)methylene)-2-methyl-5-nitro-N-(2-(piperidin-1-yl)ethyl)benzenesulfonohydrazide hydrochloride
N′-((5-Bromopyrazolo[1,5-a]pyridin-3-yl)methylene)-2-methyl-5-nitro-N-(3-(piperidin-1-yl)propyl)benzenesulfonohydrazide hydrochloride
N′-((5-Bromopyrazolo[1,5-a]pyridin-3-yl)methylene)-2-methyl-N-(3-morpholinopropyl)-5-nitrobenzenesulfonohydrazide hydrochloride
2-Methyl-N′-((5-methylpyrazolo[1,5-a]pyridin-3-yl)methylene)-5-nitrobenzenesulfonohydrazide
N′-((5-Cyanopyrazolo[1,5-a]pyridin-3-yl)methylene)-2-fluoro-N-methyl-5-nitro-benzenesulfonohydrazide
N′-((5-Cyanopyrazolo[1,5-a]pyridin-3-yl)methylene)-2-((2-(dimethylamino)ethyl)-(methyl)amino)-N-methyl-5-nitrobenzenesulfonohydrazide
N′-((5-Cyanopyrazolo[1,5-a]pyridin-3-yl)methylene)-2-(2-(dimethylamino)ethylamino)-N-methyl-5-nitrobenzenesulfonohydrazide hydrochloride
N′-((5-Cyanopyrazolo[1,5-a]pyridin-3-yl)methylene)-N-methyl-2-(2-morpholinoethylamino)-5-nitrobenzenesulfonohydrazide hydrochloride
N′-((5-Cyanopyrazolo[1,5-a]pyridin-3-yl)methylene)-N-methyl-2-(methyl(2-morpholinoethyl)amino)-5-nitrobenzenesulfonohydrazide hydrochloride
N′-((5-Cyanopyrazolo[1,5-a]pyridin-3-yl)methylene)-N-methyl-2-(methyl(2-(piperidin-1-yl)ethyl)amino)-5-nitrobenzenesulfonohydrazide hydrochloride
N′-((5-Cyanopyrazolo[1,5-a]pyridin-3-yl)methylene)-N-methyl-2-(3-morpholinopropyl-amino)-5-nitrobenzenesulfonohydrazide hydrochloride
N′-((5-Cyanopyrazolo[1,5-a]pyridin-3-yl)methylene)-N-methyl-2-(methyl(2-(methyl-amino)ethyl)amino)-5-nitrobenzenesulfonohydrazide hydrochloride
2-(2-(1H-Imidazol-4-yl)ethylamino)-N′-((5-cyanopyrazolo[1,5-a]pyridin-3-yl)methylene)-N-methyl-5-nitrobenzenesulfonohydrazide hydrochloride
N′-((5-Cyanopyrazolo[1,5-a]pyridin-3-yl)methylene)-N-methyl-5-nitro-2-(pyridin-2-yl-methylamino)benzenesulfonohydrazide hydrochloride
N′-((5-Cyanopyrazolo[1,5-a]pyridin-3-yl)methylene)-N-methyl-5-nitro-2-(pyridin-3-yl-methylamino)benzenesulfonohydrazide hydrochloride
N′-((5-Cyanopyrazolo[1,5-a]pyridin-3-yl)methylene)-N-methyl-5-nitro-2-(pyridin-4-yl-methylamino)benzenesulfonohydrazide hydrochloride
N′-((5-Cyanopyrazolo[1,5-a]pyridin-3-yl)methylene)-N-methyl-2-(methyl(pyridin-3-yl-methyl)amino)-5-nitrobenzenesulfonohydrazide hydrochloride
N′-((5-Cyanopyrazolo[1,5-a]pyridin-3-yl)methylene)-2-(2-(dimethylamino)ethoxy)-N-methyl-5-nitrobenzenesulfonohydrazide hydrochloride
N′-((5-Cyanopyrazolo[1,5-a]pyridin-3-yl)methylene)-N-methyl-2-(2-morpholinoethoxy)-5-nitrobenzenesulfonohydrazide hydrochloride
N′-((5-Cyanopyrazolo[1,5-a]pyridin-3-yl)methylene)-N-methyl-5-nitro-2-(2-(pyrrolidin-1-yl)ethoxy)benzenesulfonohydrazide hydrochloride
N′-((5-Cyanopyrazolo[1,5-a]pyridin-3-yl)methylene)-N-methyl-5-nitro-2-(pyridin-2-yl-methoxy)benzenesulfonohydrazide hydrochloride
N′-((5-Bromopyrazolo[1,5-a]pyridin-3-yl)methylene)-2-fluoro-N-methyl-5-nitro-benzenesulfonohydrazide
N′-((5-Bromopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-N-methyl-2-(2-morpholinoethoxy)-5-nitrobenzenesulfonohydrazide hydrochloride
5-Cyano-N′-((5-cyanopyrazolo[1,5-a]pyridin-3-yl)methylene)-2-fluoro-N-methyl-benzenesulfonohydrazide
5-Cyano-N′-((5-cyanopyrazolo[1,5-a]pyridin-3-yl)methylene)-2-(dimethylamino)-N-methylbenzenesulfonohydrazide
5-Cyano-N′-((5-cyanopyrazolo[1,5-a]pyridin-3-yl)methylene)-2-((2-(dimethylamino)-ethyl)(methyl)amino)-N-methylbenzenesulfonohydrazide hydrochloride
5-Cyano-N′-((5-cyanopyrazolo[1,5-a]pyridin-3-yl)methylene)-N-methyl-2-(2-morpholinoethylamino)benzenesulfonohydrazide hydrochloride
5-Cyano-N′-((5-cyanopyrazolo[1,5-a]pyridin-3-yl)methylene)-N-methyl-2-(2-morpholinoethoxy)benzenesulfonohydrazide hydrochloride
2-Chloro-N′-((5-cyanopyrazolo[1,5-a]pyridin-3-yl)methylene)-N-methyl-5-nitropyridine-3-sulfonohydrazide
N′-((5-Cyanopyrazolo[1,5-a]pyridin-3-yl)methylene)-2-((2-(dimethylamino)ethyl)-(methyl)amino)-N-methyl-5-nitropyridine-3-sulfonohydrazide hydrochloride
N′-((5-Cyanopyrazolo[1,5-a]pyridin-3-yl)methylene)-N-methyl-3-nitrobenzohydrazide
N′-((5-Cyanopyrazolo[1,5-a]pyridin-3-yl)methylene)-N,2-dimethyl-5-nitrobenzohydrazide
3-((2-Methyl-2-(2-methyl-5-nitrobenzyl)hydrazono)methyl)pyrazolo[1,5-a]pyridine-5-carbonitrile
3-(1-(2-Methyl-5-nitrophenylsulfonyl)-1H-pyrazol-3-yl)pyrazolo[1,5-a]pyridine
3-(1-(3-Nitrophenylsulfonyl)-1H-pyrazol-3-yl)pyrazolo[1,5-a]pyridine
3-(3-(Pyrazolo[1,5-a]pyridin-3-yl)-1H-pyrazol-1-ylsulfonyl)benzonitrile
5-Bromo-3-(1-(2-methyl-5-nitrophenylsulfonyl)-1H-pyrazol-3-yl)pyrazolo[1,5-a]pyridine
4-(5-Bromopyrazolo[1,5-a]pyridin-3-yl)-2-(3-nitrophenylsulfinyl)thiazole
4-(5-Bromopyrazolo[1,5-a]pyridin-3-yl)-2-(3-nitrophenylsulfonyl)thiazole
2-(5-Bromopyrazolo[1,5-a]pyridin-3-yl)-5-(2-methyl-5-nitrophenylthio)-1,3,4-oxadiazole
2-(5-Bromopyrazolo[1,5-a]pyridin-3-yl)-5-(2-methyl-5-nitrophenylsulfinyl)-1,3,4-oxadiazole
2-(5-Bromo-2-methylphenylsulfonyl)-5-(5-bromopyrazolo[1,5-a]pyridin-3-yl)-1,3,4-thiadiazole

In a seventh aspect, there is provided a compound of Formula III

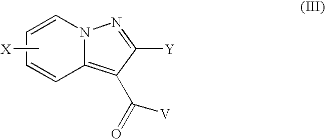

wherein V, X and Y are as defined in the fifth aspect of the invention, with the proviso that 5-methoxypyrazolo[1,5-a]pyridine-3-carboxaldehyde, 5-hydroxy-2-methylpyrazolo[1,5-a]pyridine-3-carboxaldehyde, 2,7-dimethylpyrazolo[1,5-a]pyridine-3-carboxaldehyde, 2-methylpyrazolo[1,5-a]pyridine-3-carboxaldehyde, 4-methylpyrazolo[1,5-a]pyridine-3-carboxaldehyde, pyrazolo[1,5-a]pyridine-3-carboxaldehyde, 1-(6-chloropyrazolo[1,5-a]pyridin-3-yl)ethanone, 1-(6-methylpyrazolo[1,5-a]pyridin-3-yl)ethanone, 1-(4-methylpyrazolo[1,5-a]pyridin-3-yl)ethanone and 1-pyrazolo[1,5-a]pyridin-3-ylethanone are excluded.

In one embodiment there is a compound of Formula III

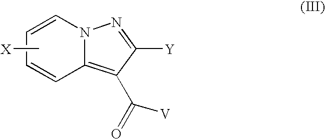

wherein X and Y are as defined in the fifth aspect and V is H or CH₃.

In one embodiment, the compound of Formula III is selected from:
5-Methylpyrazolo[1,5-a]pyridine-3-carbaldehyde
5-(Trifluoromethyl)pyrazolo[1,5-a]pyridine-3-carbaldehyde
2,5-Dimethylpyrazolo[1,5-a]pyridine-3-carbaldehyde
3-Formylpyrazolo[1,5-a]pyridine-5-carbonitrile
Methyl 3-formylpyrazolo[1,5-a]pyridine-5-carboxylate
3-Formylpyrazolo[1,5-a]pyridine-5-carboxamide
5-(2-Hydroxyethyl)pyrazolo[1,5-a]pyridine-3-carbaldehyde
2-(3-Formylpyrazolo[1,5-a]pyridin-5-yl)ethyl acetate
5-Hydroxypyrazolo[1,5-a]pyridine-3-carbaldehyde
3-Formylpyrazolo[1,5-a]pyridin-5-ylacetate
5-Aminopyrazolo[1,5-a]pyridine-3-carbaldehyde
2,2,2-Trifluoro-N-(3-formylpyrazolo[1,5-a]pyridin-5-yl)acetamide
5-Chloropyrazolo[1,5-a]pyridine-3-carbaldehyde
5-Bromopyrazolo[1,5-a]pyridine-3-carbaldehyde
5-Iodopyrazolo[1,5-a]pyridine-3-carbaldehyde
5-Vinylpyrazolo[1,5-a]pyridine-3-carbaldehyde
5-Cyclopropylpyrazolo[1,5-a]pyridine-3-carbaldehyde
5-Ethynylpyrazolo[1,5-a]pyridine-3-carbaldehyde
tert-Butyl 3-acetylpyrazolo[1,5-a]pyridin-5-ylcarbamate
1-(5-Aminopyrazolo[1,5-a]pyridin-3-yl)ethanone
1-(5-Bromopyrazolo[1,5-a]pyridin-3-yl)ethanone

In a further aspect, there is provided a compound of Formula IV

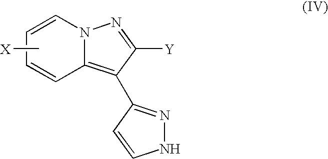

wherein X and Y are as defined in the first aspect of the invention.

In one embodiment, the compound of Formula IV is selected from:
3-(1H-Pyrazol-3-yl)pyrazolo[1,5-a]pyridine
5-Bromo-3-(1H-pyrazol-3-yl)pyrazolo[1,5-a]pyridine

In a further aspect, there is provided a compound of Formula V

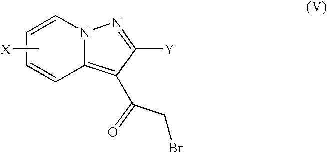

wherein X and Y are as defined in the first aspect of the invention, with the proviso that 2-bromo-1-pyrazolo[1,5-a]pyridin-3-ylethanone, 2-bromo-1-(2,5-dimethylpyrazolo[1,5-a]pyridin-3-yl)ethanone, 2-bromo-1-(2-methylpyrazolo[1,5-a]pyridin-3-yl)ethanone are excluded.

In one embodiment, the compound of Formula V is:
2-Bromo-1-(5-bromopyrazolo[1,5-a]pyridin-3-yl)ethanone.

In a further aspect, there is provided a compound of Formula VI

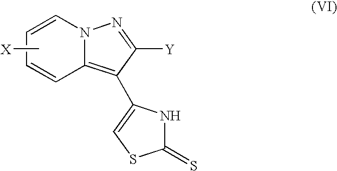

wherein X and Y are as defined in the first aspect.

In one embodiment, the compound of Formula VI is:
4-(5-Bromopyrazolo[1,5-a]pyridin-3-yl)thiazole-2(3H)thione.

In a further aspect, there is provided a compound of Formula VII

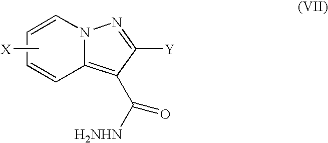

wherein X and Y are as defined in the first aspect of the invention, with the proviso that pyrazolo[1,5-a]pyridine-3-carbohydrazide is excluded.

In one embodiment, the compound of Formula VII is:
5-Bromopyrazolo[1,5-a]pyridine-3-carbohydrazide.

In a further aspect, there is provided a compound of Formula VIII

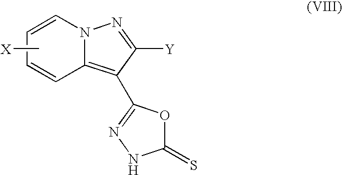

wherein X and Y are as defined in the first aspect.

In one embodiment, the compound of Formula VIII is:
5-(5-Bromopyrazolo[1,5-a]pyridin-3-yl)-1,3,4-oxadiazole-2(3H)-thione.

In a further aspect, there is provided a compound of Formula IX

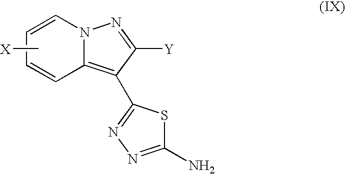

wherein X and Y are as defined in the first aspect of the invention.

In one embodiment, the compound of Formula IX is:
5-(5-Bromopyrazolo[1,5-a]pyridin-3-yl)-1,3,4-thiadiazol-2-amine.

In a further aspect, there is provided a compound of Formula X

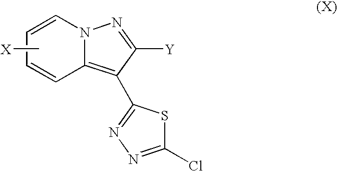

wherein X and Y are as defined in the first aspect of the invention.

In one embodiment, the compound of Formula X is:
2-(5-Bromopyrazolo[1,5-a]pyridin-3-yl)-5-chloro-1,3,4-thiadiazole.

It is to be recognised that certain compounds may exist in one or more different enantiomeric or diastereomeric forms. It is to be understood that the enantiomeric or diasteriomeric forms are included in the above aspects.

Further aspects of the present invention will become apparent from the following description given by way of example only and with reference to the accompanying synthetic schemes.

### DETAILED DESCRIPTION

The present invention broadly relates to a new class of compounds for use as agents or drugs for cancer therapy and related methods. In particular, it relates to a class of compounds that can be used as PI3K inhibitors. PI3K inhibitors are thought to be valuable for the treatment of cell proliferation disorders and particularly as anti tumour agents.

In one embodiment the compounds are broadly defined by Formula (I),

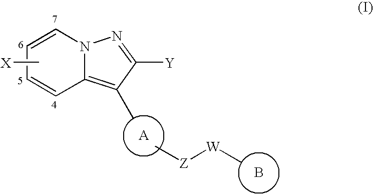

wherein;
X may represent up to two of R, F, Cl, Br, I, OR, OCOR, CONR₂, CO₂R, SO₂R, SO₂NR₂, CN, CF₃, OCF₃, NO₂, NR₂, NHCOR or optionally substituted aryl, placed at any of the available positions 4-, 5-, 6-, 7;
R may be H or C1-C6 saturated or unsaturated alkyl optionally substituted with halogen, OH, OR¹, NHR¹, NR¹₂, or optionally substituted aryl or heteroaryl, or in the case where R forms part of NR₂ this may form an optionally substituted 4-7 membered saturated ring optionally containing one additional heteroatom from the group O, S, NR²;
R¹ is H, C1-C6 saturated or unsaturated alkyl, or optionally substituted aryl or heteroaryl, or in the case when R¹ forms part of NR¹₂ this may form an optionally substituted 4-7 membered saturated ring optionally containing one additional heteroatom from the group O, S, NR²;
R² is H or C1-C6 saturated or unsaturated alkyl;

### Y may be H or CH₃;

A represents [symbol] —CH═N—N(R)- [symbol] (where [symbol] is linked to the 3-position of the pyrazole ring of formula I and [symbol] is linked to Z), or any 5-membered heterocylic ring containing up to three of the atoms S, O or N in the ring, and optionally substituted with R, as defined above.
Z represents SOₓ (where x=0-2), CH₂ or CO;
W is absent or (CH₂)_{y} where y=1, 2 or 3;
B is phenyl, naphthyl, or 5- or 6-membered heterocycle or benzoheterocycle, where the heterocylic ring contains up to two of the atoms S, O or N, optionally substituted at any available position with T, which is up to two of F, Cl, Br, I, R, OR, CONR₂, CO₂R, SO₂R, SO₂NHR, CN, CF₃, OCF₃, NO₂, NR₂, NHCOR, where R is defined as above;
or a physiologically acceptable salt or phosphate prodrug or carboxylic acid or amino acid ester prodrug thereof.

In one embodiment X is substituted at the 5-position with R, halogen, OR, OCOR, CONR₂, CO₂R, SO₂R, SO₂NR₂, CN, CF₃, OCF₃, NO₂, NR₂, NHCOR or optionally substituted aryl, where R is defined as above.

In one embodiment Z is SO₂ and W is absent.

In one embodiment B is phenyl, optionally substituted at any position with T.

In a further embodiment, A is selected from formulae IIa-IIe, where [symbol] is linked to the 3-position of the pyrazole ring of formula (I) and [symbol] is linked to Z, where R is defined as above.

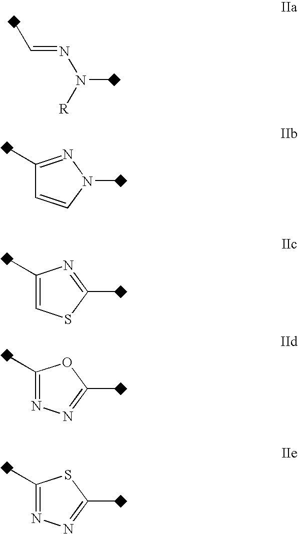

In particular, the compound of formula I as defined above can be selected from:
N,2-Dimethyl-5-nitro-N′-(pyrazolo[1,5-a]pyridin-3-ylmethylene)benzenesulfonohydrazide
N,2-Dimethyl-N′-((5-methylpyrazolo[1,5-a]pyridin-3-yl)methylene)-5-nitrobenzene-sulfonohydrazide
N,2-Dimethyl-5-nitro-N′-((5-(trifluoromethyl)pyrazolo[1,5-a]pyridin-3-yl)methylene)-benzenesulfonohydrazide
N′-((2,5-Dimethylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N,2-dimethyl-5-nitrobenzene-sulfonohydrazide
Methyl 3-((2-methyl-2-(2-methyl-5-nitrophenylsulfonyl)hydrazono)methyl)pyrazolo[1,5-a]pyridine-5-carboxylate
3-((2-Methyl-2-(2-methyl-5-nitrophenylsulfonyl)hydrazono)methyl)pyrazolo[1,5-a]pyridine-5-carboxamide
N′-((5-(2-Hydroxyethyl)pyrazolo[1,5-a]pyridin-3-yl)methylene)-N,2-dimethyl-5-nitro-benzenesulfonohydrazide
N′-((5-Methoxypyrazolo[1,5-a]pyridin-3-yl)methylene)-N,2-dimethyl-5-nitrobenzene-sulfonohydrazide
3-((2-Methyl-2-(2-methyl-5-nitrophenylsulfonyl)hydrazono)methyl)pyrazolo[1,5-a]pyridin-5-yl acetate
N′-((5-Hydroxypyrazolo[1,5-a]pyridin-3-yl)methylene)-N,2-dimethyl-5-nitrobenzene-sulfonohydrazide
N′-((5-Aminopyrazolo[1,5-a]pyridin-3-yl)methylene)-N,2-dimethyl-5-nitrobenzene-sulfonohydrazide
N′-((5-Chloropyrazolo[1,5-a]pyridin-3-yl)methylene)-N,2-dimethyl-5-nitrobenzene-sulfonohydrazide
N′-((5-Bromopyrazolo[1,5-a]pyridin-3-yl)methylene)-N,2-dimethyl-5-nitrobenzene-sulfonohydrazide
N′-((5-Iodopyrazolo[1,5-a]pyridin-3-yl)methylene)-N,2-dimethyl-5-nitrobenzene-sulfonohydrazide
N,2-Dimethyl-5-nitro-N′-((5-vinylpyrazolo[1,5-a]pyridin-3-yl)methylene)benzene-sulfonohydrazide
N′-((5-Cyclopropylpyrazolo[1,5-a]pyridin-3-yl)methylene)-N,2-dimethyl-5-nitro-benzenesulfonohydrazide
N′-((5-Ethynylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N-methyl-3-nitrobenzenesulfonohydrazide
3-Cyano-N′-((5-cyanopyrazolo[1,5-a]pyridin-3-yl)methylene)-N-methylbenzene-sulfonohydrazide
5-Cyano-N′-((5-cyanopyrazolo[1,5-a]pyridin-3-yl)methylene)-N,2-dimethylbenzene-sulfonohydrazide
N′-((5-Cyanopyrazolo[1,5-a]pyridin-3-yl)methylene)-N-methyl-3-(trifluoromethyl)-benzenesulfonohydrazide
N′-((5-Cyanopyrazolo[1,5-a]pyridin-3-yl)methylene)-N,2-dimethyl-5-(trifluoromethyl)-benzenesulfonohydrazide
N′-((5-Cyanopyrazolo[1,5-a]pyridin-3-yl)methylene)-N,2-dimethylbenzenesulfonohydrazide
N′-((5-Cyanopyrazolo[1,5-a]pyridin-3-yl)methylene)-N-methyl-4-nitrobenzenesulfonohydrazide
N′-((5-Cyanopyrazolo[1,5-a]pyridin-3-yl)methylene)-N,2-dimethyl-4-nitrobenzene-sulfonohydrazide
N′-((5-Cyanopyrazolo[1,5-a]pyridin-3-yl)methylene)-5-fluoro-N,2-dimethylbenzene-sulfonohydrazide
5-Bromo-N′-((5-cyanopyrazolo[1,5-a]pyridin-3-yl)methylene)-N,2-dimethyl-benzene-sulfonohydrazide
3-Bromo-N′-((5-cyanopyrazolo[1,5-a]pyridin-3-yl)methylene)-N-methylbenzene-sulfonohydrazide
Methyl 3-(2-((5-cyanopyrazolo[1,5-a]pyridin-3-yl)methylene)-1-methylhydrazinylsulfonyl)-4-methylbenzoate
N′-((5-Cyanopyrazolo[1,5-a]pyridin-3-yl)methylene)-N,2-dimethyl-5-(methylsulfonyl)-benzenesulfonohydrazide
N′-((5-Cyanopyrazolo[1,5-a]pyridin-3-yl)methylene)-2-ethyl-N-methyl-5-nitrobenzene-sulfonohydrazide
N′-((5-Cyanopyrazolo[1,5-a]pyridin-3-yl)methylene)-2-isopropyl-N-methyl-5-nitro-benzenesulfonohydrazide
2-Chloro-N′-((5-cyanopyrazolo[1,5-a]pyridin-3-yl)methylene)-N-methyl-5-nitro-benzenesulfonohydrazide
N′-((5-Cyanopyrazolo[1,5-a]pyridin-3-yl)methylene)-2-methoxy-N-methyl-5-nitro-benzenesulfonohydrazide
N′-((5-Cyanopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-5-cyano-N,2-dimethylbenzene-sulfonohydrazide
N′-((5-Cyanopyrazolo[1,5-a]pyridin-3-yl)methylene)-N-(2-hydroxyethyl)-2-methyl-5-nitrobenzenesulfonohydrazide
N′-((5-Bromopyrazolo[1,5-a]pyridin-3-yl)methylene)-N-(2-hydroxyethyl)-2-methyl-5-nitrobenzenesulfonohydrazide
5-Cyano-N′-((5-cyanopyrazolo[1,5-a]pyridin-3-yl)methylene)-N-(2-hydroxyethyl)-2-methylbenzenesulfonohydrazide
N-(2-Hydroxyethyl)-2-methyl-5-nitro-N′-(pyrazolo[1,5-a]pyridin-3-ylmethylene)-benzenesulfonohydrazide
N′-((5-Cyanopyrazolo[1,5-a]pyridin-3-yl)methylene)-2-methyl-5-nitrobenzenesulfonohydrazide
N-Benzyl-N′-((5-cyanopyrazolo[1,5-a]pyridin-3-yl)methylene)-2-methyl-5-nitro-benzenesulfonohydrazide
N′-((5-Cyanopyrazolo[1,5-a]pyridin-3-yl)methylene)-N-ethyl-2-methyl-5-nitrobenzene-sulfonohydrazide
N′-((5-Cyanopyrazolo[1,5-a]pyridin-3-yl)methylene)-N-(2-(diethylamino)ethyl)-2-methyl-5-nitrobenzenesulfonohydrazide
N′-((5-Cyanopyrazolo[1,5-a]pyridin-3-yl)methylene)-N-(2-(dimethylamino)ethyl)-2-methyl-5-nitrobenzenesulfonohydrazide
N′-((5-Cyanopyrazolo[1,5-a]pyridin-3-yl)methylene)-2-methyl-N-(2-morpholinoethyl)-5-nitrobenzenesulfonohydrazide
N′-((5-Cyanopyrazolo[1,5-a]pyridin-3-yl)methylene)-2-methyl-5-nitro-N-(2-(piperidin-1-yl)ethyl)benzenesulfonohydrazide
N′-((5-Cyanopyrazolo[1,5-a]pyridin-3-yl)methylene)-2-methyl-5-nitro-N-(2-(pyrrolidin-1-yl)ethyl)benzenesulfonohydrazide
N′-((5-Bromopyrazolo[1,5-a]pyridin-3-yl)methylene)-2-methyl-5-nitrobenzenesulfonohydrazide
N′-((5-Bromopyrazolo[1,5-a]pyridin-3-yl)methylene)-2-methyl-5-nitro-N-(2-(piperidin-1-yl)ethyl)benzenesulfonohydrazide hydrochloride
N′-((5-Bromopyrazolo[1,5-a]pyridin-3-yl)methylene)-2-methyl-5-nitro-N-(3-(piperidin-1-yl)propyl)benzenesulfonohydrazide hydrochloride
N′-((5-Bromopyrazolo[1,5-a]pyridin-3-yl)methylene)-2-methyl-N-(3-morpholinopropyl)-5-nitrobenzenesulfonohydrazide hydrochloride
2-Methyl-N′-((5-methylpyrazolo[1,5-a]pyridin-3-yl)methylene)-5-nitrobenzenesulfonohydrazide
N′-((5-Cyanopyrazolo[1,5-a]pyridin-3-yl)methylene)-2-fluoro-N-methyl-5-nitro-benzenesulfonohydrazide
N′-((5-Cyanopyrazolo[1,5-a]pyridin-3-yl)methylene)-2-((2-(dimethylamino)ethyl)-(methyl)amino)-N-methyl-5-nitrobenzenesulfonohydrazide
N′-((5-Cyanopyrazolo[1,5-a]pyridin-3-yl)methylene)-2-(2-(dimethylamino)ethylamino)-N-methyl-5-nitrobenzenesulfonohydrazide hydrochloride
N′-((5-Cyanopyrazolo[1,5-a]pyridin-3-yl)methylene)-N-methyl-2-(2-morpholinoethylamino)-5-nitrobenzenesulfonohydrazide hydrochloride
N′-((5-Cyanopyrazolo[1,5-a]pyridin-3-yl)methylene)-N-methyl-2-(methyl(2-morpholinoethyl)amino)-5-nitrobenzenesulfonohydrazide hydrochloride
N′-((5-Cyanopyrazolo[1,5-a]pyridin-3-yl)methylene)-N-methyl-2-(methyl(2-(piperidin-1-yl)ethyl)amino)-5-nitrobenzenesulfonohydrazide hydrochloride
N′-((5-Cyanopyrazolo[1,5-a]pyridin-3-yl)methylene)-N-methyl-2-(3-morpholinopropyl-amino)-5-nitrobenzenesulfonohydrazide hydrochloride
N′-((5-Cyanopyrazolo[1,5-a]pyridin-3-yl)methylene)-N-methyl-2-(methyl(2-(methyl-amino)ethyl)amino)-5-nitrobenzenesulfonohydrazide hydrochloride
2-(2-(1H-Imidazol-4-yl)ethylamino)-N′-((5-cyanopyrazolo[1,5-a]pyridin-3-yl)methylene)-N-methyl-5-nitrobenzenesulfonohydrazide hydrochloride
N′-((5-Cyanopyrazolo[1,5-a]pyridin-3-yl)methylene)-N-methyl-5-nitro-2-(pyridin-2-yl-methylamino)benzenesulfonohydrazide hydrochloride
N′-((5-Cyanopyrazolo[1,5-a]pyridin-3-yl)methylene)-N-methyl-5-nitro-2-(pyridin-3-yl-methylamino)benzenesulfonohydrazide hydrochloride
N′-((5-Cyanopyrazolo[1,5-a]pyridin-3-yl)methylene)-N-methyl-5-nitro-2-(pyridin-4-yl-methylamino)benzenesulfonohydrazide hydrochloride
N′-((5-Cyanopyrazolo[1,5-a]pyridin-3-yl)methylene)-N-methyl-2-(methyl(pyridin-3-yl-methyl)amino)-5-nitrobenzenesulfonohydrazide hydrochloride
N′-((5-Cyanopyrazolo[1,5-a]pyridin-3-yl)methylene)-2-(2-(dimethylamino)ethoxy)-N-methyl-5-nitrobenzenesulfonohydrazide hydrochloride
N′-((5-Cyanopyrazolo[1,5-a]pyridin-3-yl)methylene)-N-methyl-2-(2-morpholinoethoxy)-5-nitrobenzenesulfonohydrazide hydrochloride
N′-((5-Cyanopyrazolo[1,5-a]pyridin-3-yl)methylene)-N-methyl-5-nitro-2-(2-(pyrrolidin-1-yl)ethoxy)benzenesulfonohydrazide hydrochloride
N′-((5-Cyanopyrazolo[1,5-a]pyridin-3-yl)methylene)-N-methyl-5-nitro-2-(pyridin-2-yl-methoxy)benzenesulfonohydrazide hydrochloride
N′-((5-Bromopyrazolo[1,5-a]pyridin-3-yl)methylene)-2-fluoro-N-methyl-5-nitro-benzenesulfonohydrazide
N′-((5-Bromopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-N-methyl-2-(2-morpholinoethoxy)-5-nitrobenzenesulfonohydrazide hydrochloride
5-Cyano-N′-((5-cyanopyrazolo[1,5-a]pyridin-3-yl)methylene)-2-fluoro-N-methyl-benzenesulfonohydrazide
5-Cyano-N′-((5-cyanopyrazolo[1,5-a]pyridin-3-yl)methylene)-2-(dimethylamino)-N-methylbenzenesulfonohydrazide
5-Cyano-N′-((5-cyanopyrazolo[1,5-a]pyridin-3-yl)methylene)-2-((2-(dimethylamino)-ethyl)(methyl)amino)-N-methylbenzenesulfonohydrazide hydrochloride
5-Cyano-N′-((5-cyanopyrazolo[1,5-a]pyridin-3-yl)methylene)-N-methyl-2-(2-morpholinoethylamino)benzenesulfonohydrazide hydrochloride
5-Cyano-N′-((5-cyanopyrazolo[1,5-a]pyridin-3-yl)methylene)-N-methyl-2-(2-morpholinoethoxy)benzenesulfonohydrazide hydrochloride
2-Chloro-N′-((5-cyanopyrazolo[1,5-a]pyridin-3-yl)methylene)-N-methyl-5-nitropyridine-3-sulfonohydrazide
N′-((5-Cyanopyrazolo[1,5-a]pyridin-3-yl)methylene)-2-((2-(dimethylamino)ethyl)-(methyl)amino)-N-methyl-5-nitropyridine-3-sulfonohydrazide hydrochloride
N′-((5-Cyanopyrazolo[1,5-a]pyridin-3-yl)methylene)-N-methyl-3-nitrobenzohydrazide
N′-((5-Cyanopyrazolo[1,5-a]pyridin-3-yl)methylene)-N,2-dimethyl-5-nitrobenzohydrazide
3-((2-Methyl-2-(2-methyl-5-nitrobenzyl)hydrazono)methyl)pyrazolo[1,5-a]pyridine-5-carbonitrile
3-(1-(2-Methyl-5-nitrophenylsulfonyl)-1H-pyrazol-3-yl)pyrazolo[1,5-a]pyridine
3-(1-(3-Nitrophenylsulfonyl)-1H-pyrazol-3-yl)pyrazolo[1,5-a]pyridine
3-(3-(Pyrazolo[1,5-a]pyridin-3-yl)-1H-pyrazol-1-ylsulfonyl)benzonitrile
5-Bromo-3-(1-(2-methyl-5-nitrophenylsulfonyl)-1H-pyrazol-3-yl)pyrazolo[1,5-a]pyridine
4-(5-Bromopyrazolo[1,5-a]pyridin-3-yl)-2-(3-nitrophenylsulfinyl)thiazole
4-(5-Bromopyrazolo[1,5-a]pyridin-3-yl)-2-(3-nitrophenylsulfonyl)thiazole
2-(5-Bromopyrazolo[1,5-a]pyridin-3-yl)-5-(2-methyl-5-nitrophenylthio)-1,3,4-oxadiazole
2-(5-Bromopyrazolo[1,5-a]pyridin-3-yl)-5-(2-methyl-5-nitrophenylsulfinyl)-1,3,4-oxadiazole
2-(5-Bromo-2-methylphenylsulfonyl)-5-(5-bromopyrazolo[1,5-a]pyridin-3-yl)-1,3,4-thiadiazole

It is appreciated that compounds of Formula I may occur in different geometric and enantiomeric forms, and that both pure forms and mixtures of these separate isomers are included, and any physiologically acceptable salts or phosphate or carboxylic acid or amino acid ester prodrugs thereof.

In another embodiment there is provided a method of cancer prevention or therapy for treating cancers including the step of administering a compound of Formula I as defined above to a subject in need thereof. Further, there is provided the use of a compound of Formula I in the manufacture of a medicament for the treatment of cancer.

In one embodiment, the method includes administration of a compound of Formula I together with administering one or more suitable chemotherapeutic agents and/or therapies. These agents and therapies can be of any suitable type as would be well known to a skilled person, however a non-limiting list would include agents and therapies selected from:
Alkylation agents (eg cisplatin, carboplatin)
Antimetabolites (eg methotrexate, 5-FU)
Antitumour antibiotics (eg adriamymycin, bleomycin)
Antitumour vegetable alkaloids (eg taxol, etoposide)
Antitumor hormones (eg dexamethasone, tamoxifen)
Antitumour immunological agents (eg interferon α, β, γ)
Radiation therapy
Surgery

In one embodiment the method of therapy further includes the step of administering one or more chemotherapeutic agents and/or therapies to the subject before, during or after the administration of the compound of Formula I as defined.

While these compounds will typically be used in cancer prevention or cancer therapy of human subjects, they can be used to target cancer cells in other warm blooded animal subjects such as other primates, farm animals such as cattle, and sports animals and pets such as horses, dogs, and cats.

In other embodiments there is provided a pharmaceutical composition including a compound of Formula I as defined above, and a pharmaceutically acceptable excipient, adjuvant, carrier, buffer or stabiliser. In one embodiment the pharmaceutical composition will take the form of a tablet, capsule, powder, or liquid form. In one embodiment the composition will be suitable for oral or parenteral administration.

The pharmaceutically acceptable excipient, adjuvant, carrier, buffer or stabiliser can be of any known type and should be non-toxic and should not interfere with the efficacy of the active ingredient. The precise nature of the carrier or other material will depend on the route of administration, which may be oral, or by injection, such as cutaneous, subcutaneous, or intravenous injection.

The pharmaceutical compositions of the invention formulated for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin. Such formulation issues will be well known to a person skilled in the formulation art.

Where pharmaceutical compositions may be formulated for intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as Sodium Chloride injection, Ringer's injection, Lactated Ringer's injection. Preservatives, stabilisers, buffers antioxidants and/or other additives as are known to be suitable for such use may be included as required.

In a further embodiment, the pharmaceutical compositions also include one or more chemotherapeutic agents as defined above.

In another embodiment there is provided the use of a compound of Formula I in the manufacture of a medicament for the treatment of cancer.

In one embodiment the medicament is in tablet, capsule, powder or liquid form. In a particular embodiment the medicament will be suitable for oral or parenteral administration. Typically, the medicament will be formulated as described above.

In another embodiment there is provided a method of making a compound of Formula I as defined above, the method including the step of modifying pyrazolo[1,5-a]pyridine-3-carbonyl compound of Formula III

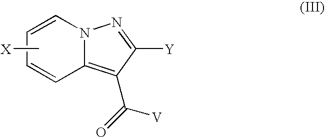

wherein variables X and Y are as defined above for Formula I and V is H, CH₃ or alkoxy. In one embodiment, where V is alkoxy it is ethoxy (OEt).

In one embodiment of the method for preparing compound of Formula I from compound of Formula III, where X and Y are as defined above for Formula I and V is CH₃, the method involves one of the following:

(i) reaction with DMFdma then cyclisation with hydrazine, followed by sulfonylation as illustrated in Scheme 27 below; or
(ii) bromination then cyclisation with dithiocarbamate, coupling with a boronic acid followed by oxidation as illustrated in Schemes 28 and 29 below.

In a further embodiment of the method for preparing compound of Formula I from compound of Formula III, where X and Y are as defined above for Formula I and V is OEt, the method involves one of the following:
(i) reaction with hydrazine then cyclisation with CS₂, coupling with a boronic acid followed by oxidation as illustrated in Scheme 30 below; or
(ii) basic hydrolysis, then reaction with thiosemicarbazide, cyclisation with H₂SO₄, followed by diazotisation and chlorination, then substitution with a sulfinate salt as illustrated in Schemes 3 and 31 below.

In a further embodiment, the method of preparing compound of Formula I from compound of Formula III involves reaction with DMFdma then cyclisation with hydrazine and proceeds via an intermediate of Formula IV

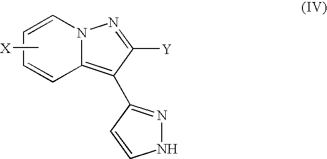

wherein variables X and Y are as defined above for Formula I.

In one embodiment when X and Y are as defined above for Formula I and V is H, the method for preparing compound of Formula I from compound of Formula III involves one of the following:
(i) condensation with a hydrazine followed by sulfonylation as illustrated in Schemes 15 and 21 below;
(ii) condensation with a sulfonohydrazide followed by alkylation as illustrated in Schemes 16 and 22 below;
(iii) reaction with methylhydrazine sulphate followed by acylation as illustrated in Scheme 24 below;
(iv) condensation with a hydrazine as illustrated in Scheme 25 below; or
(v) condensation with methylhydrazine sulphate followed by sulfonylation and then nucleophilic substitution as illustrated in Scheme 23 below.

In one embodiment the method of preparing a compound of Formula I from a compound of Formula III include a route via the:
(a) preparation of compounds of Formula V and VI substantially in accordance with Scheme 28 (defined later herein); or
(b) preparation of compounds of Formula VII and VIII substantially in accordance with Scheme 30 (defined later herein); or
(c) preparation of compounds of Formula IX and X substantially in accordance with Scheme 31 (defined later herein); wherein

Formula V to X (wherein variables X and Y are as defined for Formula I) are:

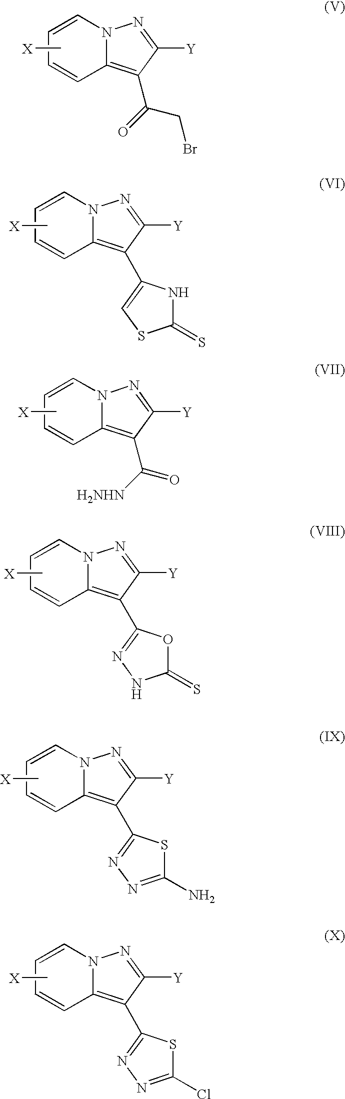

In another embodiment there is provided a compound of Formula I obtained by the methods according to the present invention described herein. In one particular embodiment, the compound of Formula I obtained by such methods is selected from one or more of the following:
N,2-Dimethyl-5-nitro-N′-(pyrazolo[1,5-a]pyridin-3-ylmethylene)benzenesulfonohydrazide
N,2-Dimethyl-N′-((5-methylpyrazolo[1,5-a]pyridin-3-yl)methylene)-5-nitrobenzene-sulfonohydrazide
N,2-Dimethyl-5-nitro-N′-((5-(trifluoromethyl)pyrazolo[1,5-a]pyridin-3-yl)methylene)-benzenesulfonohydrazide
N′-((2,5-Dimethylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N,2-dimethyl-5-nitrobenzene-sulfonohydrazide
Methyl 3-((2-methyl-2-(2-methyl-5-nitrophenylsulfonyl)hydrazono)methyl)pyrazolo[1,5-a]pyridine-5-carboxylate
3-((2-Methyl-2-(2-methyl-5-nitrophenylsulfonyl)hydrazono)methyl)pyrazolo[1,5-a]pyridine-5-carboxamide
N′-((5-(2-Hydroxyethyl)pyrazolo[1,5-a]pyridin-3-yl)methylene)-N,2-dimethyl-5-nitro-benzenesulfonohydrazide
N′-((5-Methoxypyrazolo[1,5-a]pyridin-3-yl)methylene)-N,2-dimethyl-5-nitrobenzene-sulfonohydrazide
3-((2-Methyl-2-(2-methyl-5-nitrophenylsulfonyl)hydrazono)methyl)pyrazolo[1,5-a]pyridin-5-yl acetate
N′-((5-Hydroxypyrazolo[1,5-a]pyridin-3-yl)methylene)-N,2-dimethyl-5-nitrobenzene-sulfonohydrazide
N′-((5-Aminopyrazolo[1,5-a]pyridin-3-yl)methylene)-N,2-dimethyl-5-nitrobenzene-sulfonohydrazide
N′-((5-Chloropyrazolo[1,5-a]pyridin-3-yl)methylene)-N,2-dimethyl-5-nitrobenzene-sulfonohydrazide
N′-((5-Bromopyrazolo[1,5-a]pyridin-3-yl)methylene)-N,2-dimethyl-5-nitrobenzene-sulfonohydrazide
N′-((5-Iodopyrazolo[1,5-a]pyridin-3-yl)methylene)-N,2-dimethyl-5-nitrobenzene-sulfonohydrazide
N,2-Dimethyl-5-nitro-N′-((5-vinylpyrazolo[1,5-a]pyridin-3-yl)methylene)benzene-sulfonohydrazide
N′-((5-Cyclopropylpyrazolo[1,5-a]pyridin-3-yl)methylene)-N,2-dimethyl-5-nitro-benzenesulfonohydrazide
N′-((5-Ethynylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N-methyl-3-nitrobenzenesulfonohydrazide
3-Cyano-N′-((5-cyanopyrazolo[1,5-a]pyridin-3-yl)methylene)-N-methylbenzene-sulfonohydrazide
5-Cyano-N′-((5-cyanopyrazolo[1,5-a]pyridin-3-yl)methylene)-N,2-dimethylbenzene-sulfonohydrazide
N′-((5-Cyanopyrazolo[1,5-a]pyridin-3-yl)methylene)-N-methyl-3-(trifluoromethyl)-benzenesulfonohydrazide
N′-((5-Cyanopyrazolo[1,5-a]pyridin-3-yl)methylene)-N,2-dimethyl-5-(trifluoromethyl)-benzenesulfonohydrazide
N′-((5-Cyanopyrazolo[1,5-a]pyridin-3-yl)methylene)-N,2-dimethylbenzenesulfonohydrazide
N′-((5-Cyanopyrazolo[1,5-a]pyridin-3-yl)methylene)-N-methyl-4-nitrobenzenesulfonohydrazide
N′-((5-Cyanopyrazolo[1,5-a]pyridin-3-yl)methylene)-N,2-dimethyl-4-nitrobenzene-sulfonohydrazide
N′-((5-Cyanopyrazolo[1,5-a]pyridin-3-yl)methylene)-5-fluoro-N,2-dimethylbenzene-sulfonohydrazide
5-Bromo-N′-((5-cyanopyrazolo[1,5-a]pyridin-3-yl)methylene)-N,2-dimethyl-benzene-sulfonohydrazide
3-Bromo-N′-((5-cyanopyrazolo[1,5-a]pyridin-3-yl)methylene)-N-methylbenzene-sulfonohydrazide
Methyl 3-(2-((5-cyanopyrazolo[1,5-a]pyridin-3-yl)methylene)-1-methylhydrazinylsulfonyl)-4-methylbenzoate
N′-((5-Cyanopyrazolo[1,5-a]pyridin-3-yl)methylene)-N,2-dimethyl-5-(methylsulfonyl)-benzenesulfonohydrazide
N′-((5-Cyanopyrazolo[1,5-a]pyridin-3-yl)methylene)-2-ethyl-N-methyl-5-nitrobenzene-sulfonohydrazide
N′-((5-Cyanopyrazolo[1,5-a]pyridin-3-yl)methylene)-2-isopropyl-N-methyl-5-nitro-benzenesulfonohydrazide
2-Chloro-N′-((5-cyanopyrazolo[1,5-a]pyridin-3-yl)methylene)-N-methyl-5-nitro-benzenesulfonohydrazide
N′-((5-Cyanopyrazolo[1,5-a]pyridin-3-yl)methylene)-2-methoxy-N-methyl-5-nitro-benzenesulfonohydrazide
N′-((5-Cyanopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-5-cyano-N,2-dimethylbenzene-sulfonohydrazide
N′-((5-Cyanopyrazolo[1,5-a]pyridin-3-yl)methylene)-N-(2-hydroxyethyl)-2-methyl-5-nitrobenzenesulfonohydrazide
N′-((5-Bromopyrazolo[1,5-a]pyridin-3-yl)methylene)-N-(2-hydroxyethyl)-2-methyl-5-nitrobenzenesulfonohydrazide
5-Cyano-N′-((5-cyanopyrazolo[1,5-a]pyridin-3-yl)methylene)-N-(2-hydroxyethyl)-2-methylbenzenesulfonohydrazide
N-(2-Hydroxyethyl)-2-methyl-5-nitro-N′-(pyrazolo[1,5-a]pyridin-3-ylmethylene)-benzenesulfonohydrazide
N′-((5-Cyanopyrazolo[1,5-a]pyridin-3-yl)methylene)-2-methyl-5-nitrobenzenesulfonohydrazide
N-Benzyl-N′-((5-cyanopyrazolo[1,5-a]pyridin-3-yl)methylene)-2-methyl-5-nitro-benzenesulfonohydrazide
N′-((5-Cyanopyrazolo[1,5-a]pyridin-3-yl)methylene)-N-ethyl-2-methyl-5-nitrobenzene-sulfonohydrazide
N′-((5-Cyanopyrazolo[1,5-a]pyridin-3-yl)methylene)-N-(2-(diethylamino)ethyl)-2-methyl-5-nitrobenzenesulfonohydrazide
N′-((5-Cyanopyrazolo[1,5-a]pyridin-3-yl)methylene)-N-(2-(dimethylamino)ethyl)-2-methyl-5-nitrobenzenesulfonohydrazide
N′-((5-Cyanopyrazolo[1,5-a]pyridin-3-yl)methylene)-2-methyl-N-(2-morpholinoethyl)-5-nitrobenzenesulfonohydrazide
N′-((5-Cyanopyrazolo[1,5-a]pyridin-3-yl)methylene)-2-methyl-5-nitro-N-(2-(piperidin-1-yl)ethyl)benzenesulfonohydrazide
N′-((5-Cyanopyrazolo[1,5-a]pyridin-3-yl)methylene)-2-methyl-5-nitro-N-(2-(pyrrolidin-1-yl)ethyl)benzenesulfonohydrazide
N′-((5-Bromopyrazolo[1,5-a]pyridin-3-yl)methylene)-2-methyl-5-nitrobenzenesulfonohydrazide
N′-((5-Bromopyrazolo[1,5-a]pyridin-3-yl)methylene)-2-methyl-5-nitro-N-(2-(piperidin-1-yl)ethyl)benzenesulfonohydrazide hydrochloride
N′-((5-Bromopyrazolo[1,5-a]pyridin-3-yl)methylene)-2-methyl-5-nitro-N-(3-(piperidin-1-yl)propyl)benzenesulfonohydrazide hydrochloride
N′-((5-Bromopyrazolo[1,5-a]pyridin-3-yl)methylene)-2-methyl-N-(3-morpholinopropyl)-5-nitrobenzenesulfonohydrazide hydrochloride
2-Methyl-N′-((5-methylpyrazolo[1,5-a]pyridin-3-yl)methylene)-5-nitrobenzenesulfonohydrazide
N′-((5-Cyanopyrazolo[1,5-a]pyridin-3-yl)methylene)-2-fluoro-N-methyl-5-nitro-benzenesulfonohydrazide
N′-((5-Cyanopyrazolo[1,5-a]pyridin-3-yl)methylene)-2-((2-(dimethylamino)ethyl)-(methyl)amino)-N-methyl-5-nitrobenzenesulfonohydrazide
N′-((5-Cyanopyrazolo[1,5-a]pyridin-3-yl)methylene)-2-(2-(dimethylamino)ethylamino)-N-methyl-5-nitrobenzenesulfonohydrazide hydrochloride
N′-((5-Cyanopyrazolo[1,5-a]pyridin-3-yl)methylene)-N-methyl-2-(2-morpholinoethylamino)-5-nitrobenzenesulfonohydrazide hydrochloride
N′-((5-Cyanopyrazolo[1,5-a]pyridin-3-yl)methylene)-N-methyl-2-(methyl(2-morpholinoethyl)amino)-5-nitrobenzenesulfonohydrazide hydrochloride
N′-((5-Cyanopyrazolo[1,5-a]pyridin-3-yl)methylene)-N-methyl-2-(methyl(2-(piperidin-1-yl)ethyl)amino)-5-nitrobenzenesulfonohydrazide hydrochloride
N′-((5-Cyanopyrazolo[1,5-a]pyridin-3-yl)methylene)-N-methyl-2-(3-morpholinopropyl-amino)-5-nitrobenzenesulfonohydrazide hydrochloride
N′-((5-Cyanopyrazolo[1,5-a]pyridin-3-yl)methylene)-N-methyl-2-(methyl(2-(methyl-amino)ethyl)amino)-5-nitrobenzenesulfonohydrazide hydrochloride
2-(2-(1H-Imidazol-4-yl)ethylamino)-N′-((5-cyanopyrazolo[1,5-a]pyridin-3-yl)methylene)-N-methyl-5-nitrobenzenesulfonohydrazide hydrochloride
N′-((5-Cyanopyrazolo[1,5-a]pyridin-3-yl)methylene)-N-methyl-5-nitro-2-(pyridin-2-yl-methylamino)benzenesulfonohydrazide hydrochloride
N′-((5-Cyanopyrazolo[1,5-a]pyridin-3-yl)methylene)-N-methyl-5-nitro-2-(pyridin-3-yl-methylamino)benzenesulfonohydrazide hydrochloride
N′-((5-Cyanopyrazolo[1,5-a]pyridin-3-yl)methylene)-N-methyl-5-nitro-2-(pyridin-4-yl-methylamino)benzenesulfonohydrazide hydrochloride
N′-((5-Cyanopyrazolo[1,5-a]pyridin-3-yl)methylene)-N-methyl-2-(methyl(pyridin-3-yl-methyl)amino)-5-nitrobenzenesulfonohydrazide hydrochloride
N′-((5-Cyanopyrazolo[1,5-a]pyridin-3-yl)methylene)-2-(2-(dimethylamino)ethoxy)-N-methyl-5-nitrobenzenesulfonohydrazide hydrochloride
N′-((5-Cyanopyrazolo[1,5-a]pyridin-3-yl)methylene)-N-methyl-2-(2-morpholinoethoxy)-5-nitrobenzenesulfonohydrazde hydrochloride
N′-((5-Cyanopyrazolo[1,5-a]pyridin-3-yl)methylene)-N-methyl-5-nitro-2-(2-(pyrrolidin-1-yl)ethoxy)benzenesulfonohydrazide hydrochloride
N′-((5-Cyanopyrazolo[1,5-a]pyridin-3-yl)methylene)-N-methyl-5-nitro-2-(pyridin-2-yl-methoxy)benzenesulfonohydrazide hydrochloride
N′-((5-Bromopyrazolo[1,5-a]pyridin-3-yl)methylene)-2-fluoro-N-methyl-5-nitro-benzenesulfonohydrazide
N′-((5-Bromopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-N-methyl-2-(2-morpholinoethoxy)-5-nitrobenzenesulfonohydrazide hydrochloride
5-Cyano-N′-((5-cyanopyrazolo[1,5-a]pyridin-3-yl)methylene)-2-fluoro-N-methyl-benzenesulfonohydrazide
5-Cyano-N′-((5-cyanopyrazolo[1,5-a]pyridin-3-yl)methylene)-2-(dimethylamino)-N-methylbenzenesulfonohydrazide
5-Cyano-N′-((5-cyanopyrazolo[1,5-a]pyridin-3-yl)methylene)-2-((2-(dimethylamino)-ethyl)(methyl)amino)-N-methylbenzenesulfonohydrazide hydrochloride
5-Cyano-N′-((5-cyanopyrazolo[1,5-a]pyridin-3-yl)methylene)-N-methyl-2-(2-morpholinoethylamino)benzenesulfonohydrazide hydrochloride
5-Cyano-N′-((5-cyanopyrazolo[1,5-a]pyridin-3-yl)methylene)-N-methyl-2-(2-morpholinoethoxy)benzenesulfonohydrazide hydrochloride
2-Chloro-N′-((5-cyanopyrazolo[1,5-a]pyridin-3-yl)methylene)-N-methyl-5-nitropyridine-3-sulfonohydrazide
N′-((5-Cyanopyrazolo[1,5-a]pyridin-3-yl)methylene)-2-((2-(dimethylamino)ethyl)-(methyl)amino)-N-methyl-5-nitropyridine-3-sulfonohydrazide hydrochloride
N′-((5-Cyanopyrazolo[1,5-a]pyridin-3-yl)methylene)-N-methyl-3-nitrobenzohydrazide
N′-((5-Cyanopyrazolo[1,5-a]pyridin-3-yl)methylene)-N,2-dimethyl-5-nitrobenzohydrazide
3-((2-Methyl-2-(2-methyl-5-nitrobenzyl)hydrazono)methyl)pyrazolo[1,5-a]pyridine-5-carbonitrile
3-(1-(2-Methyl-5-nitrophenylsulfonyl)-1H-pyrazol-3-yl)pyrazolo[1,5-a]pyridine
3-(1-(3-Nitrophenylsulfonyl)-1H-pyrazol-3-yl)pyrazolo[1,5-a]pyridine
3-(3-(Pyrazolo[1,5-a]pyridin-3-yl)-1H-pyrazol-1-ylsulfonyl)benzonitrile
5-Bromo-3-(1-(2-methyl-5-nitrophenylsulfonyl)-1H-pyrazol-3-yl)pyrazolo[1,5-a]pyridine
4-(5-Bromopyrazolo[1,5-a]pyridin-3-yl)-2-(3-nitrophenylsulfinyl)thiazole
4-(5-Bromopyrazolo[1,5-a]pyridin-3-yl)-2-(3-nitrophenylsulfonyl)thiazole
2-(5-Bromopyrazolo[1,5-a]pyridin-3-yl)-5-(2-methyl-5-nitrophenylthio)-1,3,4-oxadiazole
2-(5-Bromopyrazolo[1,5-a]pyridin-3-yl)-5-(2-methyl-5-nitrophenylsulfinyl)-1,3,4-oxadiazole
2-(5-Bromo-2-methylphenylsulfonyl)-5-(5-bromopyrazolo[1,5-a]pyridin-3-yl)-1,3,4-thiadiazole

Another embodiment provides a compound of Formula III

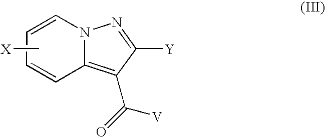

wherein V, X and Y are as defined herein before with the proviso that 5-methoxypyrazolo-[1,5-a]pyridine-3-carboxaldehyde, 5-hydroxy-2-methylpyrazolo[1,5-a]pyridine-3-carboxaldehyde, 2,7-dimethylpyrazolo[1,5-a]pyridine-3-carboxaldehyde, 2-methyl-pyrazolo[1,5-a]pyridine-3-carboxaldehyde, 4-methylpyrazolo[1,5-a]pyridine-3-carboxaldehyde, pyrazolo[1,5-a]pyridine-3-carboxaldehyde, 1-(6-chloropyrazolo[1,5-a]pyridin-3-yl)ethanone, 1-(6-methylpyrazolo[1,5-a]pyridin-3-yl)ethanone, 1-(4-methylpyrazolo[1,5-a]pyridin-3-yl)ethanone and 1-pyrazolo[1,5-a]pyridin-3-ylethanone are excluded.

In one embodiment, in Formula III X and Y are as defined for formula I and V is H or CH₃.

In one embodiment, the compound of Formula III is selected from:
5-Methylpyrazolo[1,5-a]pyridine-3-carbaldehyde
5-(Trifluoromethyl)pyrazolo[1,5-a]pyridine-3-carbaldehyde
2,5-Dimethylpyrazolo[1,5-a]pyridine-3-carbaldehyde
3-Formylpyrazolo[1,5-a]pyridine-5-carbonitrile
Methyl 3-formylpyrazolo[1,5-a]pyridine-5-carboxylate
3-Formylpyrazolo[1,5-a]pyridine-5-carboxamide
5-(2-Hydroxyethyl)pyrazolo[1,5-a]pyridine-3-carbaldehyde
2-(3-Formylpyrazolo[1,5-a]pyridin-5-yl)ethyl acetate
5-Hydroxypyrazolo[1,5-a]pyridine-3-carbaldehyde
3-Formylpyrazolo[1,5-a]pyridin-5-yl acetate
5-Aminopyrazolo[1,5-a]pyridine-3-carbaldehyde
2,2,2-Trifluoro-N-(3-formylpyrazolo[1,5-a]pyridin-5-yl)acetamide
5-Chloropyrazolo[1,5-a]pyridine-3-carbaldehyde
5-Bromopyrazolo[1,5-a]pyridine-3-carbaldehyde
5-Iodopyrazolo[1,5-a]pyridine-3-carbaldehyde
5-Vinylpyrazolo[1,5-a]pyridine-3-carbaldehyde
5-Cyclopropylpyrazolo[1,5-a]pyridine-3-carbaldehyde
5-Ethynylpyrazolo[1,5-a]pyridine-3-carbaldehyde
tert-Butyl 3-acetylpyrazolo[1,5-a]pyridin-5-ylcarbamate
1-(5-Aminopyrazolo[1,5-a]pyridin-3-yl)ethanone
1-(5-Bromopyrazolo[1,5-a]pyridin-3-yl)ethanone

Another embodiment provides a compound of Formula IV

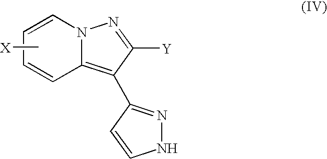

wherein X and Y are as defined above for Formula 1.

In one embodiment the compound of Formula IV is selected from:
3-(1H-Pyrazol-3-yl)pyrazolo[1,5-a]pyridine.
5-Bromo-3-(1H-pyrazol-3-yl)pyrazolo[1,5-a]pyridine

Another embodiment provides a compound of Formula V

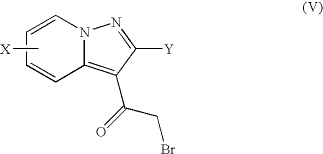

wherein X and Y are as defined above for Formula I, with the proviso that 2-bromo-1-pyrazolo[1,5-a]pyridin-3-ylethanone, 2-bromo-1-(2,5-dimethylpyrazolo[1,5-a]pyridin-3-yl)ethanone, 2-bromo-1-(2-methylpyrazolo[1,5-a]pyridin-3-yl)ethanone are excluded.

In one embodiment the compound of Formula V is:
2-Bromo-1-(5-bromopyrazolo[1,5-a]pyridin-3-yl)ethanone.

Another embodiment provides a compound of Formula VI

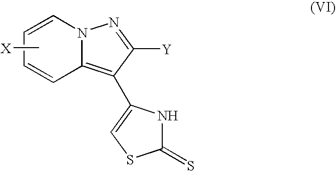

wherein X and Y are as defined above for Formula I.

In one embodiment the compound of Formula VI is:
4-(5-Bromopyrazolo[1,5-a]pyridin-3-yl)thiazole-2(3H)-thione.

A further embodiment provides a compound of Formula VII

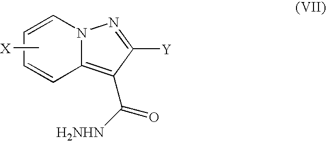

wherein X and Y are as defined above for Formula I, with the proviso that pyrazolo[1,5-a]pyridine-3-carbohydrazide is excluded.

In one embodiment of the invention the compound of Formula VII is:
5-Bromopyrazolo[1,5-a]pyridine-3-carbohydrazide.

A further embodiment provides a compound of Formula VIII

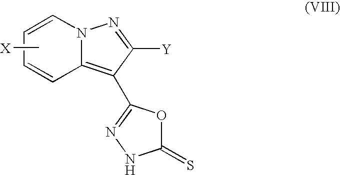

wherein X and Y are as defined above for Formula I.

In one embodiment the compound of Formula VIII is:
5-(5-Bromopyrazolo[1,5-a]pyridin-3-yl)-1,3,4-oxadiazole-2(3H)-thione.

Still further embodiments provide a compound of Formula IX

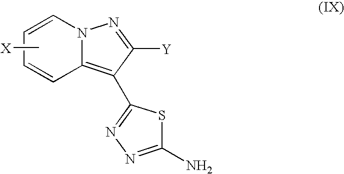

wherein X and Y are as defined above for Formula I.

In one embodiment the compound of Formula IX is:
5-(5-Bromopyrazolo[1,5-a]pyridin-3-yl)-1,3,4-thiadiazol-2-amine.

Further embodiments provide a compound of Formula X

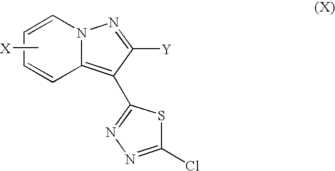

wherein X and Y are as defined above for Formula I.

In one embodiment the compound of Formula X is:
2-(5-Bromopyrazolo[1,5-a]pyridin-3-yl)-5-chloro-1,3,4-thiadiazole.

It is to be recognised that certain compounds may exist in one or more different enantiomeric or diastereomeric forms. It is to be understood that the enantiomeric or diasteriomeric forms are included in the above aspects of the invention.

The term halo or halogen group used throughout the specification is to be taken as meaning a fluoro, chloro, bromo or iodo group.

The term physiologically acceptable salt used throughout the specification is to be taken as meaning any suitable acid or base derived salt and, in particular, those formed from hydrochloric, sulfuric, phosphoric, acetic, citric, oxalic, malonic, salicylic, malic, fumaric, succinic, ascorbic, maleic, methanesulfonic, isoethonic acids and the like and potassium carbonate sodium or potassium hydroxide ammonia, triethylamine, triethanolamine and the like.

The term prodrug as used herein means any compound which releases an active parent drug according to formula (I) in vivo when such prodrug is administered to a subject. Prodrugs of a compound of formula (I) are prepared by modifying functional groups present in the compound of formula (I) in such a way that the modifications may be cleaved in vivo to release the parent compound. In particular embodiments of the invention prodrugs include phosphate prodrugs of phenols or alcohols, or carboxylic acid ester or amino acid ester prodrugs. Such prodrugs may be made by any number of standard methods recognised in the art. However, by way of example, phosphate prodrugs may be prepared by methods similar to those described by G. S. Gill et al., Org. Prep. Proc. Int. 2006, 38 (6), 604, and amino acid ester prodrugs may be prepared by methods similar to those described by L. Ribeiro et al., Arch. Pharm. 2007, 340, 32.

### Methods for Preparing Compounds of Formula I.

The substituted pyrazolo[1,5-a]pyridine compounds of the invention can be conveniently synthesised from pyrazolo[1,5-a]pyridine-3-carbonyl compound of Formula III, by several different routes, depending on the substituents and/or functionality as shown in Scheme 1:

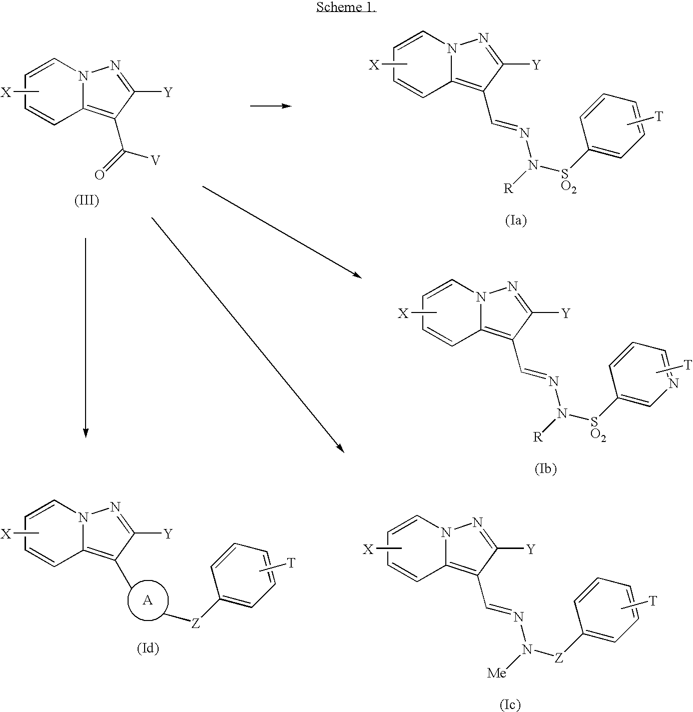

Nine suitable routes are:
(i) condensation with a hydrazine followed by sulfonylation (for compounds of Formula Ia and Ib, see Schemes 15 and 21);
(ii) condensation with a sulfonohydrazide followed by alkylation (alternative method for compounds of Formula Ia, see Schemes 16 and 22);
(iii) reaction with methylhydrazine sulphate followed by acylation (for compounds of Formula Ic where Z=CO, see Scheme 24);
(iv) reaction with an alkyl hydrazine (for compounds of Formula Ic where Z=CH₂, see Scheme 25);
(v) reaction with methylhydrazine sulphate followed by sulfonylation and then nucleophilic substitution (for compounds of Formula Ia and Ib, see Scheme 23);
(vi) reaction with DMFdma then cyclisation with hydrazine, followed by sulfonylation (for compounds of Formula Id where A is a pyrazole ring, see Scheme 27);
(vii) bromination then cyclisation with dithiocarbamate, coupling with a boronic acid followed by oxidation (for compounds of Formula Id where A is a thiazole ring, see Schemes 28 and 29);
(viii) reaction with hydrazine then cyclisation with CS₂, coupling with a boronic acid followed by oxidation (for compounds of Formula Id where A is a 1,3,4-oxadiazole ring, see Scheme 30); or
(ix) reaction with thiosemicarbazide then cyclisation with H₂SO₄, followed by diazotisation and chlorination, then substitution with a sulfinate salt (for compounds of Formula Id where A is a 1,3,4-thiadiazole ring, see Scheme 31).

### Methods for Preparing Compounds of Formula Ia and Ib

In Scheme 2, intermediates of type 3 can be made by N-amination of pyridine 1 using a suitable O-substituted hydroxylamine such as O-(mesitylsulfonyl)hydroxylamine or O-(2,4-dinitrophenyl)hydroxylamine to form N-aminopyridinium 2. Cyclisation under basic conditions with a suitable alkyne forms substituted pyrazolo[1,5-a]pyridines 3.

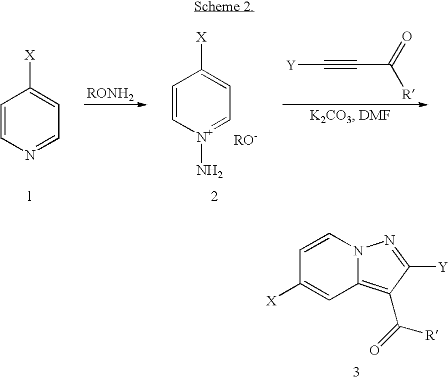

In Scheme 3, ester 4 can be converted to aldehyde 7 by hydrolysis of ester 4 under basic conditions to form carboxylic acid 5. Reduction to alcohol 6 can be achieved by NaBH₄ reduction of the intermediate imidazolide, and then re-oxidation to aldehyde with MnO₂ affords aldehyde 7.

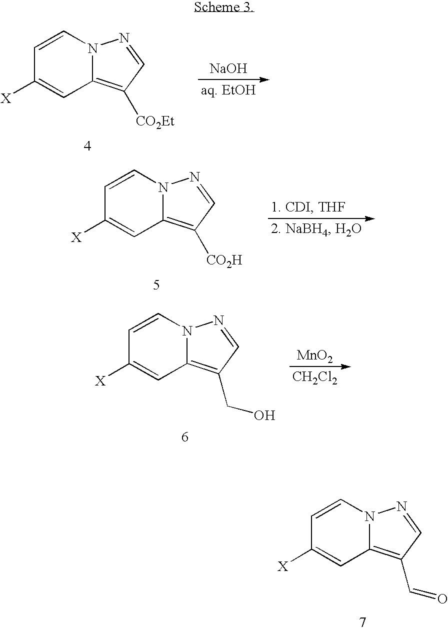

In Scheme 4, aldehyde 10 can be made by LiAlH₄ reduction of ester 8 to afford alcohol 9, which can be re-oxidised to aldehyde 10 with MnO₂.

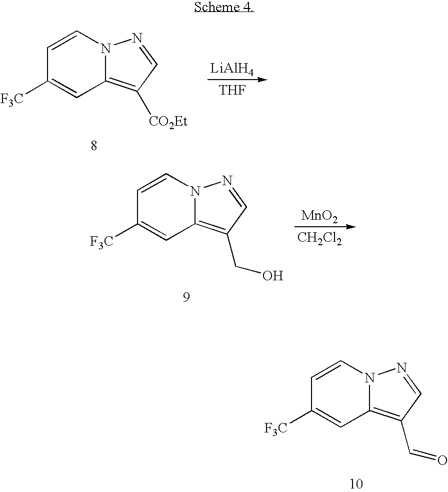

In Scheme 5, decarboxylation of ester 11 by refluxing in 40% H₂SO₄ forms intermediate 12, which is then converted to aldehyde 13 with a Vilsmeier reaction.

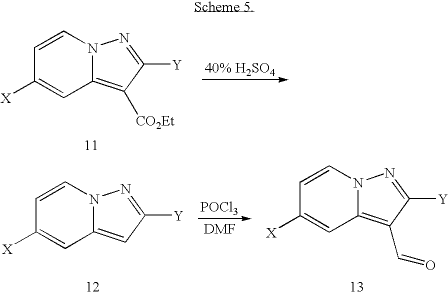

Scheme 6 shows the synthesis of aldehyde 17. It can be prepared by decarboxylation of diester 14 to leave carboxylic acid 15. Conversion to carboxamide 16 is achieved by CDI activation of the carboxylic acid followed by reaction with aqueous NH₃. Vilsmeier conditions then install the aldehyde and dehydrate the carboxamide to form nitrile 17.

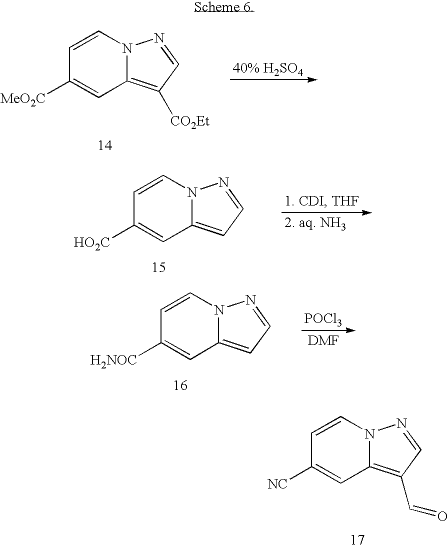

In Scheme 7, carboxylic acid 15 can be esterified by refluxing in methanol containing a catalytic amount of HCl, and then Vilsmeier reaction forms aldehyde 19. Basic hydrolysis of the ester affords carboxylic acid 20, which can then be converted to carboxamide 21 by activation with SOCl₂ followed by reaction with NH₃.

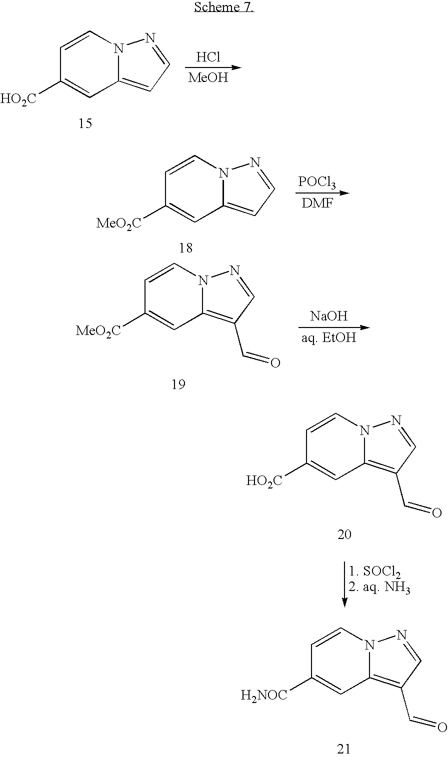

Scheme 8 shows the synthesis of aldehyde 26. Decarboxylation of ester 22 by refluxing in 40% H₂SO₄ affords 23. Protection of the primary alcohol with Ac₂O followed by a Vilsmeier reaction gives aldehyde 25, which can be deprotected with NaOH to afford 26.

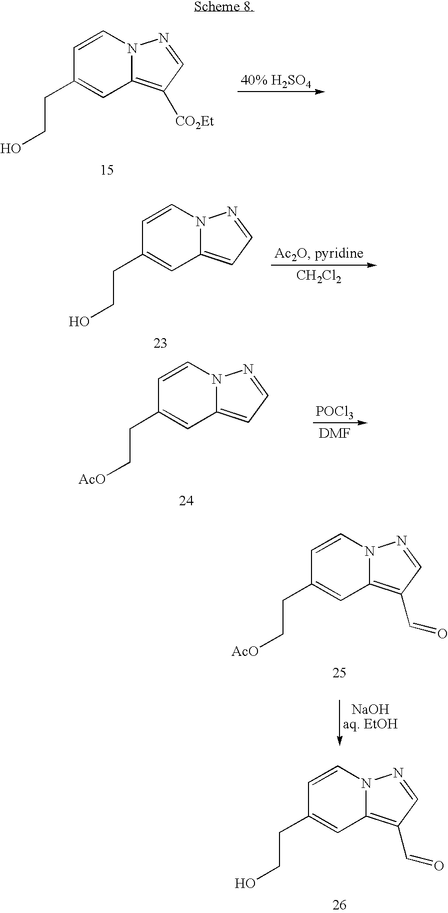

Scheme 9 shows the synthesis of aldehyde 29. Ester 27 is decarboxylated and demethylated by refluxing in 40% H₂SO₄. Methylation of phenol 28 using iodomethane and K₂CO₃ affords methyl ether 29.

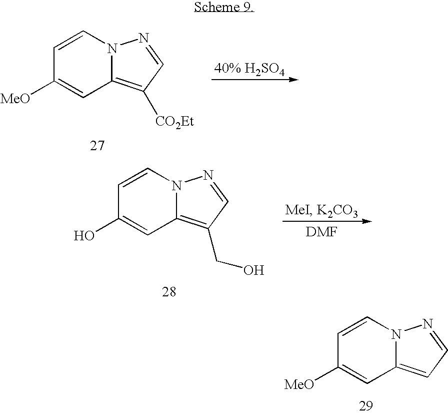

In Scheme 10, phenol 28 undergoes a Vilsmeier reaction to form 30. Acetylation with Ac₂O then affords 31.

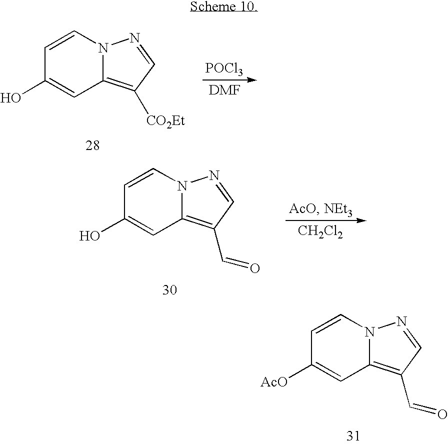

In Scheme 11, aldehyde 37 is made by TFA deprotection of Boc-protected amine 32, followed by decarboxylation by refluxing in 40% H₂SO₄ to afford amine 34. Protection as the trifluoroacetamide was achieved with TFAA, and then Vilsmeier reaction installed the aldehyde group with concomitant deprotection of the amine group (36). TFAA again was used to form the trifluoroacetamide 37.

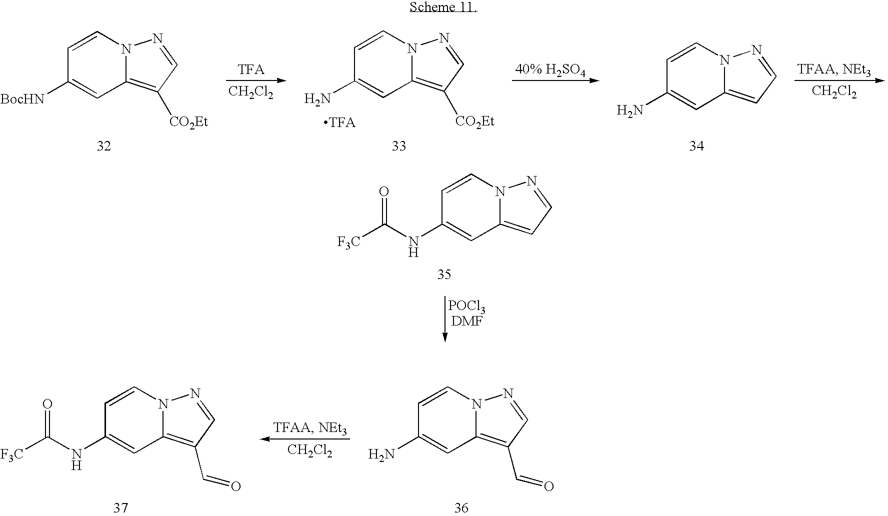

Esters of type 38 where X=Cl, Br, I can be prepared according to Scheme 12.

Diazotisation of amine 33 and reaction with a copper salt affords halogenated heterocycle 38.

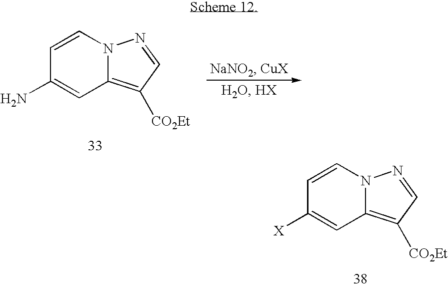

An alternative procedure to pyrazolo[1,5-a]pyridine 39 is depicted in Scheme 13.

Diazotisation of amine 34 and reaction with a copper salt affords halogenated heterocycle 39.

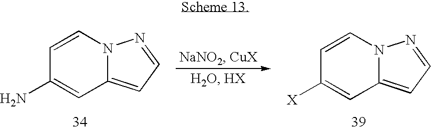

In Scheme 14, the synthesis of aldehydes 41 and 42 are depicted. Either a Stille coupling of bromide 40 with a tributyltin reagent affords 41, or a Sonogashira coupling with ethynyl-trimethylsilane affords aldehyde 42.

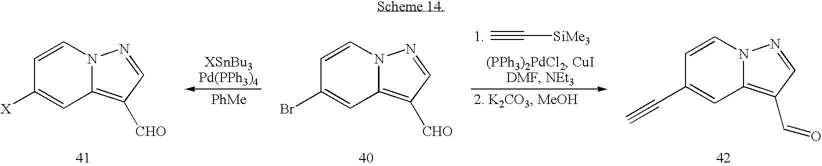

In Scheme 15, compounds of Formula Ia can be prepared from aldehyde 13 by initial condensation with methylhydrazine sulfate with a base such as NaHCO₃ or 2,6-lutidine in a solvent such as methanol followed by sulfonylation without isolation of intermediate 43 to form 44.

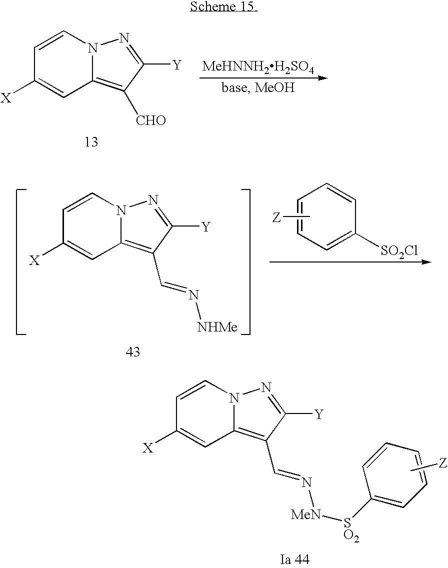

Alternatively in Scheme 16, the initial condensation of aldehyde 13 can be carried out using sulfonohydrazide 45 in methanol, and then methylation of 46 using an ethereal solution of CH₂N₂ affords 47.

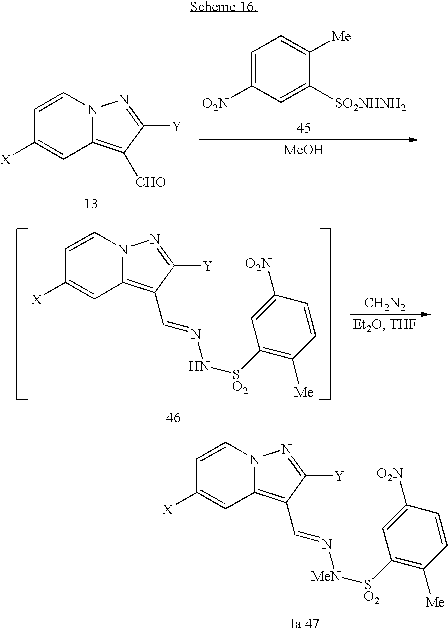

Scheme 17 shows the acetate cleavage of phenolic ester 48 with NaHCO₃ in aqueous methanol to form phenol 49.

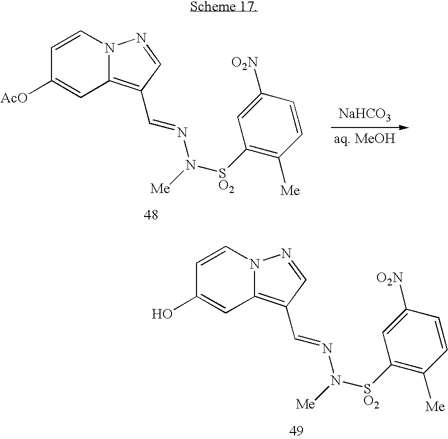

Deprotection of trifluoroacetamide 50 to amine 51 with Na₂CO₃ is shown in Scheme 18.

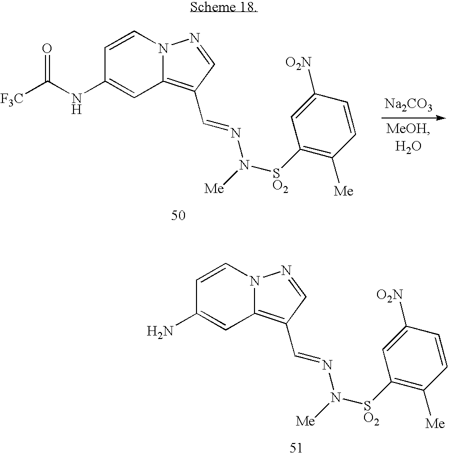

Scheme 19 shows the synthesis of sulfonyl chloride 53 from aniline 52 via reaction of the intermediate diazonium with SO₂ and CuCl₂.

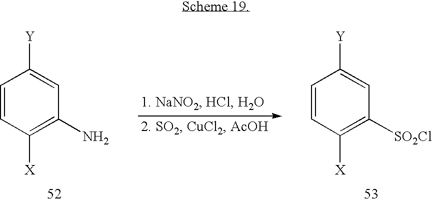

An alternative synthesis of sulfonyl chloride 53 is shown in Scheme 20, by the reaction of a para-substituted benzene 54 with ClSO₃H. Additionally, when Y=CO₂H, reaction with SOCl₂ followed by MeOH affords methyl ester 55.

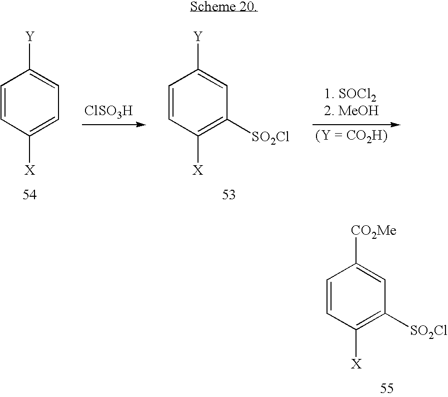

Aldehyde 13 can be converted to sulfonohydrazide 56 (Scheme 21) by initial reaction with 2-hydroxyethylhydrazine, followed by sulfonylation with a sulfonyl chloride under basic conditions.

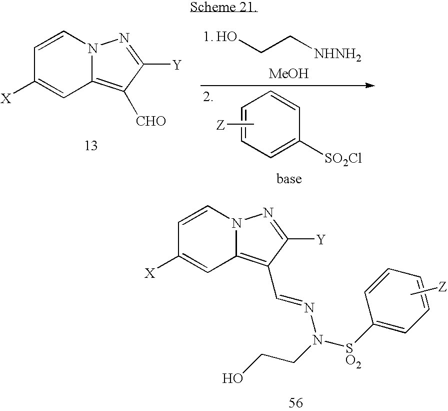

In Scheme 22, sulfonohydrazide 46 can be alkylated by initial deprotonation with NaH followed by alkylation with a suitable electrophile to give 57, or by alkylation with an amine-containing electrophile in the presence of Cs₂CO₃ to afford 58.

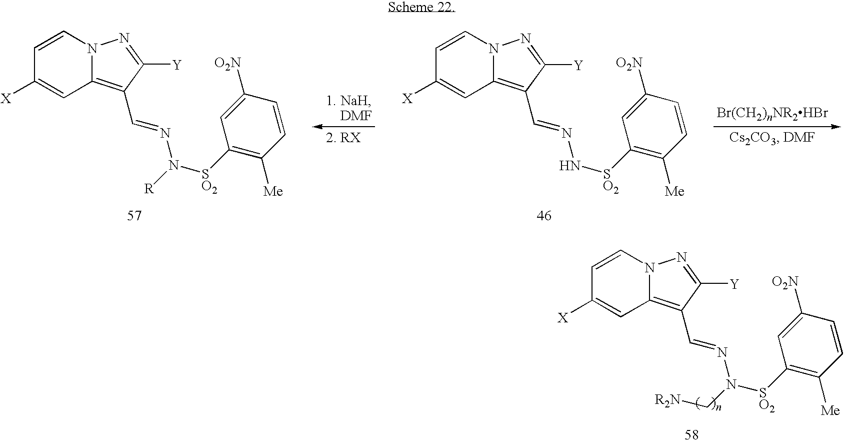

In Scheme 23, fluorobenzene 59 can be substituted by a primary or secondary amine in THF to form amine 60, or by an alcohol with NaH in THF to form ether 61.

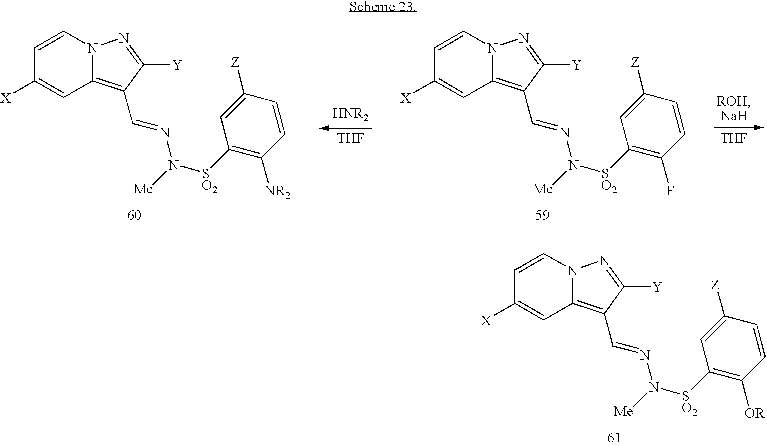

### Method for Preparing Compounds of Formula Ic

In Scheme 24, compounds of Formula Ic can be made from aldehyde 13 by reaction with methylhydrazine sulfate followed by acylation without isolation of intermediate 43 to form amide 62.

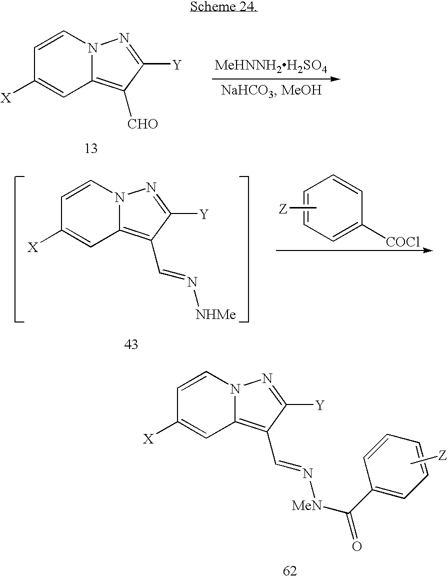

In Scheme 25, chloride 63 was substituted with methylhydrazine to form hydrazine 64. Condensation with aldehyde 13 afforded hydrazone 65.

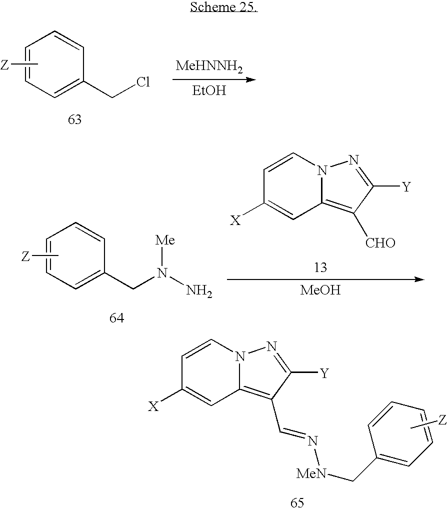

### Method for Preparing Compounds of Formula Id

In Scheme 26, bromo compound 68 can be prepared by deprotection of tert-butyl carbamate 66 with TFA to form amine 67. Subsequently, a Sandmeyer reaction using a copper bromide salt, leads to the formation of bromide 68.

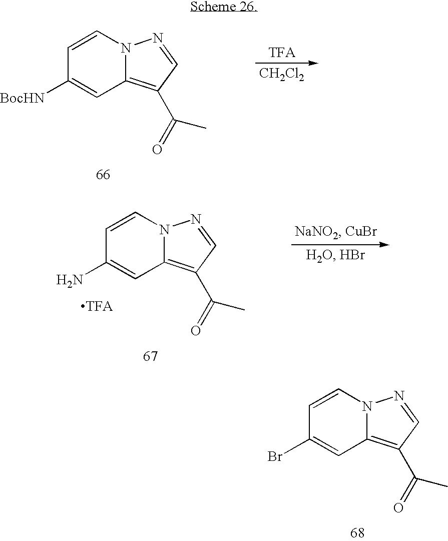

In Scheme 27, compounds of Formula Ia can be prepared by reacting ketone 69 with N,N-dimethylformamide dimethyl acetal, followed by a cyclisation with hydrazine to form pyrazole 70. Sulfonylation to form 71 can be carried out with a range of arylsulfonyl chlorides using NEt₃ as the base.

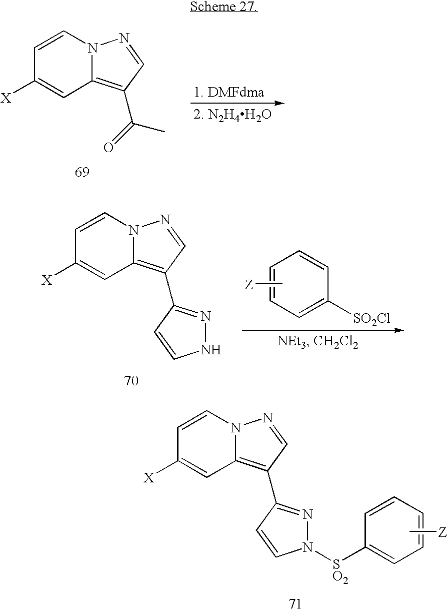

In Scheme 28, methyl ketone 69 is reacted with Br₂ in AcOH to give bromoketone 72. Cyclisation with ammonium dithiocarbamate affords thione 73, and copper catalysed coupling with a boronic acid affords substituted thiazole 74.

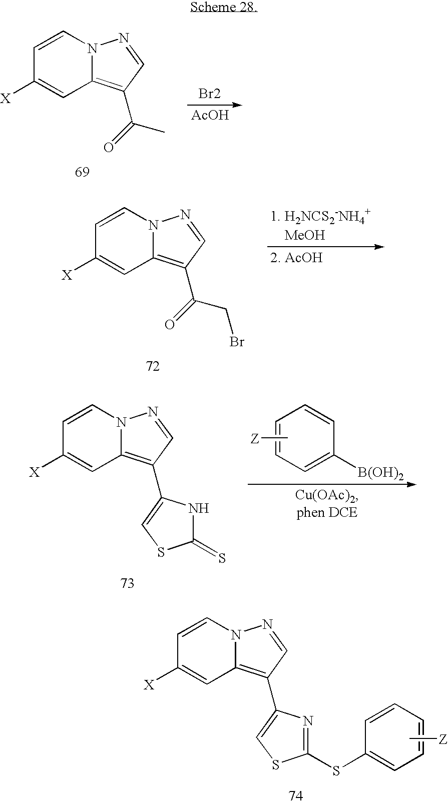

In Scheme 29, sulfide 74 is oxidised to sulfoxide 75 with oxone in aqueous MeOH, or to sulfone 76 with MMPP in CH₂Cl₂.

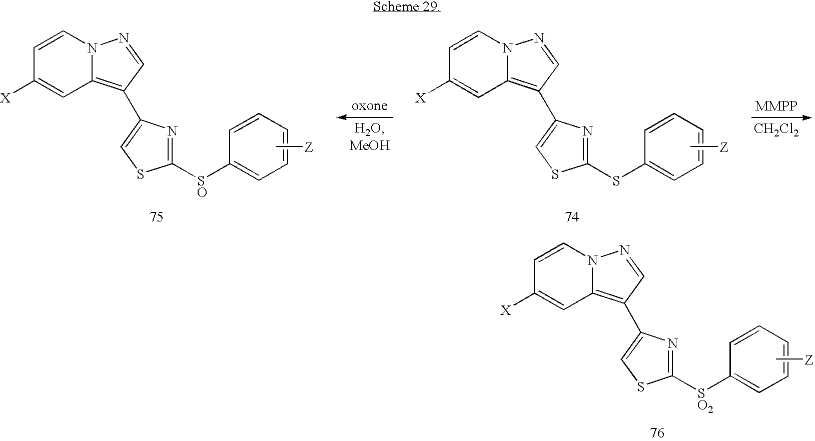

In Scheme 30, ester 38 was converted to thione 78 by reaction of intermediate acylhydrazide 77 with CS₂ followed by acidic cyclisation. Copper catalysed coupling with a boronic acid afforded sulfide 79, and then oxidation gave sulfoxide 80.

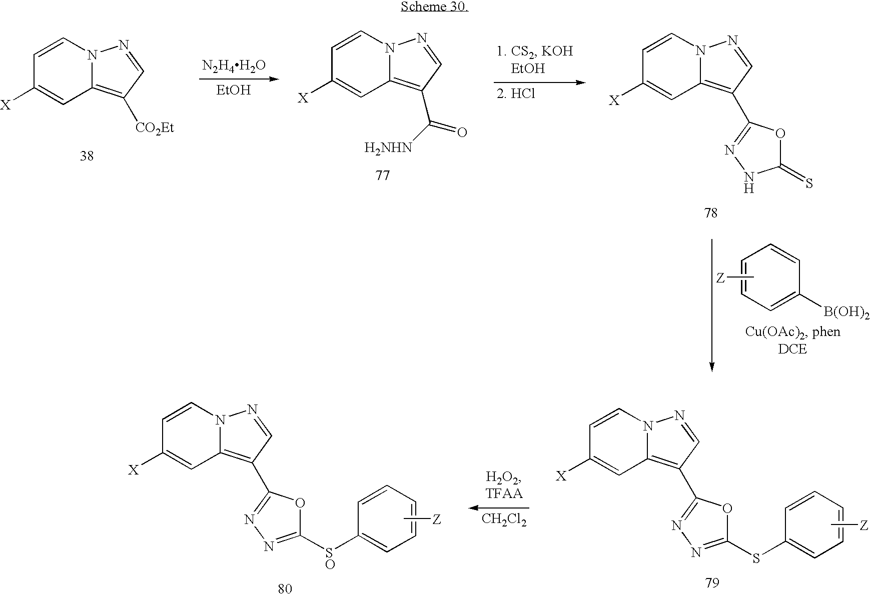

In Scheme 31, carboxylic acid 5 is converted to thiadiazole 81 by reaction of the intermediate acid chloride with thiosemicarbazide followed by acidic cyclisation. Diazotisation then introduces the chloride substituent, and then substitution with a sulfinate salt affords sulfone 83.

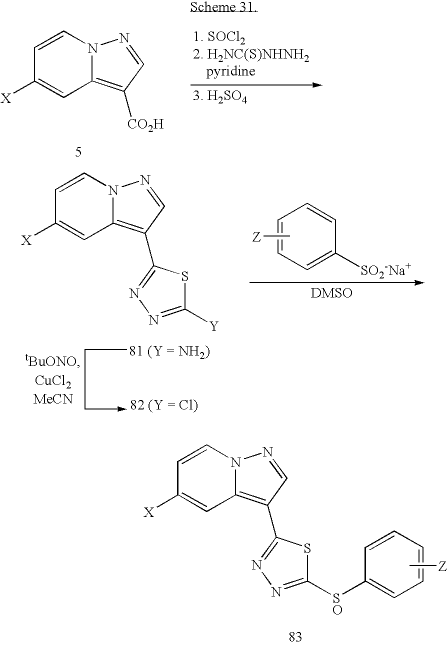

### EXAMPLES

The present invention will be described in more detail by referring to the following examples but is not deemed to be limited thereto.

Table 1 gives examples of compounds representative of the invention, and preparable by the methods outlined in Schemes 2-21.

TABLE 1

| | |
|---|---|
| 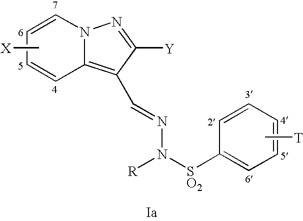 | 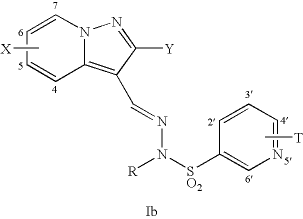 |
| 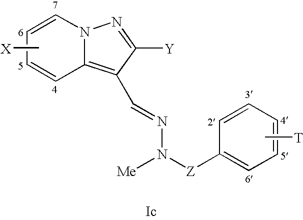 | 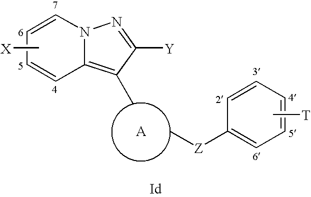 |

| No. | Fm | X | Y | R | T | A | Z | Formula^{a} |
|---|---|---|---|---|---|---|---|---|
| E1 | Ia | H | H | Me | 2′-Me, 5′-NO₂ | | | C₁₆H₁₅N₅O₄S |
| E2 | Ia | 5-Me | H | Me | 2′-Me, 5′-NO₂ | | | C₁₇H₁₇N₅O₄S |
| E3 | Ia | 5-CF₃ | H | Me | 2′-Me, 5′-NO₂ | | | C₁₇H₁₄F₃N₅O₄S |
| E4 | Ia | 5-Me | Me | Me | 2′-Me, 5′-NO₂ | | | C₁₈H₁₉N₅O₄S |
| E5 | Ia | 5-CN | H | Me | 2′-Me, 5′-NO₂ | | | C₁₇H₁₄N₆O₄S |
| E6 | Ia | 5-CO₂Me | H | Me | 2′-Me, 5′-NO₂ | | | C₁₈H₁₇N₅O₆S |
| E7 | Ia | 5-CONH₂ | H | Me | 2′-Me, 5′-NO₂ | | | C₁₇H₁₆N₆O₅S |
| E8 | Ia | 5- | H | Me | 2′-Me, 5′-NO₂ | | | C₁₈H₁₉N₅O₅S |
| | | (CH₂)₂OH | | | | | | |
| E9 | Ia | 5-OMe | H | Me | 2′-Me, 5′-NO₂ | | | C₁₇H₁₇N₅O₅S |
| E10 | Ia | 5-OCOMe | H | Me | 2′-Me, 5′-NO₂ | | | C₁₈H₁₇N₅O₆S |
| E11 | Ia | 5-OH | H | Me | 2′-Me, 5′-NO₂ | | | C₁₆H₁₅N₅O₅S |
| E12 | Ia | 5-NH₂ | H | Me | 2′-Me, 5′-NO₂ | | | C₁₆H₁₆N₆O₄S |
| E13 | Ia | 5-Cl | H | Me | 2′-Me, 5′-NO₂ | | | C₁₆H₁₄ClN₅O₄S |
| E14 | Ia | 5-Br | H | Me | 2′-Me, 5′-NO₂ | | | C₁₆H₁₄BrN₅O₄S |
| E15 | Ia | 5-I | H | Me | 2′-Me, 5′-NO₂ | | | C₁₆H₁₄IN₅O₄S |
| E16 | Ia | 5-C═CH₂ | H | Me | 2′-Me, 5′-NO₂ | | | C₁₈H₁₇N₅O₄S |
| E17 | Ia | 5-^{c}Pr | H | Me | 2′-Me, 5′-NO₂ | | | C₁₉H₁₉N₅O₄S |
| E18 | Ia | 5-CCH | H | Me | 2′-Me, 5′-NO₂ | | | C₁₈H₁₅N₅O₄S |
| EI9 | Ia | 5-CN | H | Me | 3′-NO₂ | | | C₁₆H₁₂N₆O₄S |
| E20 | Ia | 5-CN | H | Me | 3′-CN | | | C₁₇H₁₂N₆O₂S |
| E21 | Ia | 5-CN | H | Me | 2′-Me, 5′-CN | | | C₁₈H₁₄N₆O₂S |
| E22 | Ia | 5-CN | H | Me | 3′-CF₃ | | | C₁₇H₁₂F₃N₅O₂S |
| E23 | Ia | 5-CN | H | Me | 2′-Me, 5′-CF₃ | | | C₁₈H₁₄F₃N₅O₂S |
| E24 | Ia | 5-CN | H | Me | 2′-Me | | | C₁₇H₁₅N₅O₂S |
| E25 | Ia | 5-CN | H | Me | 4′-NO₂ | | | C₁₆H₁₂N₆O₄S |
| E26 | Ia | 5-CN | H | Me | 2′-Me, 4′-NO₂ | | | C₁₇H₁₄N₆O₄S |
| E27 | Ia | 5-CN | H | Me | 2′-Me, 5′-F | | | C₁₇H₁₄FN₅O₂S |
| E28 | Ia | 5-CN | H | Me | 2′-Me, 5′-Br | | | C₁₇H₁₄BrN₅O₂S |
| E29 | Ia | 5-CN | H | Me | 3′-Br | | | C₁₆H₁₂BrN₅O₂S |
| E30 | Ia | 5-CN | H | Me | 2′-Me, 5′-CO₂Me | | | C₁₉H₁₇N₅O₄S |
| E31 | Ia | 5-CN | H | Me | 2′-Me, 5′-SO₂Me | | | C₁₈H₁₇N₅O₄S₂ |
| E32 | Ia | 5-CN | H | Me | 2′-Et, 5′-NO₂ | | | C₁₈H₁₆N₆O₄S |
| E33 | Ia | 5-CN | H | Me | 2′-ⁱPr, 5′-NO₂ | | | C₁₉H₁₈N₆O₄S |
| E34 | Ia | 5-CN | H | Me | 2′-Cl, 5′-NO₂ | | | C₁₈H₁₁ClN₆O₄S |
| E35 | Ia | 5-CN | H | Me | 2′-OMe, 5′-NO₂ | | | C₁₇H₁₄N₆O₅S |
| E36 | Ia | 5-CN | H | Me | 2′-NMe₂, 5′-NO₂ | | | C₁₈H₁₇N₇O₄S |
| E37 | Ia | 5-Br | H | Me | 2′-Me, 5′-CN | | | C₁₇H₁₄BrN₅O₂S |
| E38 | Ia | 5-CN | H | (CH₂)₂OH | 2′-Me, 5′-NO₂ | | | C₁₈H₁₆N₆O₅S |
| E39 | Ia | 5-Br | H | (CH₂)₂OH | 2′-Me, 5′-NO₂ | | | C₁₇H₁₆BrN₅O₅S |
| E40 | Ia | 5-CN | H | (CH₂)₂OH | 2′-Me, 5′-CN | | | C₁₉H₁₆N₆O₃S |
| E41 | Ia | H | H | (CH₂)₂OH | 2′-Me, 5′-NO₂ | | | C₁₇H₁₇N₅O₅S |
| E42 | Ia | 5-CN | H | H | 2′-Me, 5′-NO₂ | | | C₁₆H₁₂N₆O₄S |
| E43 | Ia | 5-CN | H | CH₂Ph | 2′-Me, 5′-NO₂ | | | C₂₃H₁₈N₆O₄S |
| E44 | Ia | 5-CN | H | Et | 2′-Me, 5′-NO₂ | | | C₁₈H₁₆N₆O₄S |
| E45 | Ia | 5-CN | H | (CH₂)₂NEt₂ | 2′-Me, 5′-NO₂ | | | C₂₂H₂₅N₇O₄S |
| E46 | Ia | 5-CN | H | (CH₂)₂NMe₂ | 2′-Me, 5′-NO₂ | | | C₂₀H₂₁N₇O₄S |
| E47 | Ia | 5-CN | H | (CH₂)₂(morph) | 2′-Me, 5′-NO₂ | | | C₂₂H₂₃N₇O₅S |
| E48 | Ia | 5-CN | H | (CH₂)₂(piperid) | 2′-Me, 5′-NO₂ | | | C₂₃H₂₅N₇O₄S |
| E49 | Ia | 5-CN | H | (CH₂)₂(pyrrol) | 2′-Me, 5′-NO₂ | | | C₂₂H₂₃N₇O₄S |
| E50 | Ia | 5-Br | H | H | 2′-Me, 5′-NO₂ | | | C₁₅H₁₂BrN₅O₄S |
| E51 | Ia | 5-Br | H | (CH₂)₂(piperid) | 2′-Me, 5′-NO₂ | | | C₂₂H₂₅BrN₆O₄S•HCl |
| E52 | Ia | 5-Br | H | (CH₂)₃(piperid) | 2′-Me, 5′-NO₂ | | | C₂₃H₂₇BrN₆O₄S•HCl |
| E53 | Ia | 5-Br | H | (CH₂)₃(morph) | 2′-Me, 5′-NO₂ | | | C₂₂H₂₅BrN₆O₅S•HCl |
| E54 | Ia | 5-Me | H | H | 2′-Me, 5′-NO₂ | | | C₁₆H₁₅N₅O₄S |
| E55 | Ia | 5-CN | H | Me | 2′-F, 5′-NO₂ | | | C₁₆H₁₁FN₆O₄S |
| E56 | Ia | 5-CN | H | Me | 2′-NMe(CH₂)₂NMe₂, | | | C₂₁H₂₄N₈O₄S |
| | | | | | 5′-NO₂ | | | |
| E57 | Ia | 5-CN | H | Me | 2′-NH(CH₂)₂NMe₂, | | | C₂₀H₂₂N₈O₄S•HCl |
| | | | | | 5′-NO₂ | | | |
| E58 | Ia | 5-CN | H | Me | 2′-NH(CH₂)₂(morph), | | | C₂₂H₂₄N₈O₅S•HCl |
| | | | | | 5′-NO₂ | | | |
| E59 | Ia | 5-CN | H | Me | 2′- | | | C₂₃H₂₆N₈O₅S•HCl |
| | | | | | NMe(CH₂)₂(morph), | | | |
| | | | | | 5′-NO₂ | | | |
| E60 | Ia | 5-CN | H | Me | 2′-NMe(CH₂)₂(pip), | | | C₂₄H₂₈N₈O₄S•HCl |
| | | | | | 5′-NO₂ | | | |
| E61 | Ia | 5-CN | H | Me | 2′-NH(CH₂)₃(morph), | | | C₂₃H₂₆N₈O₅S•HCl |
| E62 | Ia | 5-CN | H | Me | 2′-NMe(CH₂)₂NHMe, | | | C₂₀H₂₂N₈O₄S•HCl |
| | | | | | 5′-NO₂ | | | |
| E63 | Ia | 5-CN | H | Me | 2′-NH(CH₂)₂(4-imid), | | | C₂₁H₁₉N₉O₄S•HCl |
| | | | | | 5′-NO₂ | | | |
| E64 | Ia | 5-CN | H | Me | 2′-NHCH₂(2-py), 5′- | | | C₂₂H₁₈N₈O₄S•HCl |
| | | | | | NO₂ | | | |
| E65 | Ia | 5-CN | H | Me | 2′-NHCH₂(3-py), 5′- | | | C₂₂H₁₈N₈O₄S•Cl |
| | | | | | NO₂ | | | |
| E66 | Ia | 5-CN | H | Me | 2′-NHCH₂(4-py), 5′- | | | C₂₂H₁₈N₈O₄S•HCl |
| | | | | | NO₂ | | | |
| E67 | Ia | 5-CN | H | Me | 2′-N(Me)CH₂(3-py), | | | C₂₃H₂₀N₈O₄S•HCl |
| | | | | | 5′-NO₂ | | | |
| E68 | Ia | 5-CN | H | Me | 2′-O(CH₂)₂NMe₂, 5′- | | | C₂₀H₂₁N₇O₅S•HCl |
| | | | | | NO₂ | | | |
| E69 | Ia | 5-CN | H | Me | 2′-O(CH₂)₂(morph), | | | C₂₂H₂₃N₇O₆S•HCl |
| | | | | | 5′-NO₂ | | | |
| E70 | Ia | 5-CN | H | Me | 2′-O(CH₂)₂(pyrrol), 5′- | | | C₂₂H₂₃N₇O₅S•HCl |
| | | | | | NO₂ | | | |
| E71 | Ia | 5-CN | H | Me | 2′-OCH₂(2-py), 5′- | | | C₂₂H₁₇N₇O₅S•HCl |
| | | | | | NO₂ | | | |
| E72 | Ia | 5-Br | H | Me | 2′-F, 5′-NO₂ | | | C₁₅H₁₁BrFN₅O₄S |
| E73 | Ia | 5-Br | H | Me | 2′-NMe₂, 5′-NO₂ | | | C₁₇H₁₇BrN₆O₄S |
| E74 | Ia | 5-Br | H | Me | 2′-O(CH₂)₂(morph), | | | C₂₁H₂₃BrN₆O₆S•HCl |
| | | | | | 5′-NO₂ | | | |
| E75 | Ia | 5-CN | H | Me | 2′-F, 5′-CN | | | C₁₇H₁₁FN₆O₂S |
| E76 | Ia | 5-CN | H | Me | 2′-NMe₂, 5′-CN | | | C₁₉H₁₇N₇O₂S |
| E77 | Ia | 5-CN | H | Me | 2′-NMe(CH₂)₂NMe_{2,} | | | C₂₂H₂₄N₈O₂S•HCl |
| | | | | | 5′-CN | | | |
| E78 | Ia | 5-CN | H | Me | 2′-NH(CH₂)₂(morph), | | | C₂₃H₂₄N₈O₃S•HCl |
| | | | | | 5′-CN | | | |
| E79 | Ia | 5-CN | H | Me | 2′-O(CH₂)₂(morph), | | | C₂₃H₂₃N₇O₄S•HCl |
| | | | | | 5′-CN | | | |
| E80 | Ib | 5-CN | H | Me | 2′-Cl, 5′-NO₂ | | | C₁₅H₁₀ClN₇O₄S |
| E81 | Ib | 5-CN | H | Me | 2′-NMe(CH₂)₂NMe₂, | | | C₂₀H₂₃N₆O₄S•HCl |
| | | | | | 5′-NO₂ | | | |
| E82 | Ic | 5-CN | H | | 3′-NO₂ | | CO | C₁₇H₁₂N₆O₃ |
| E83 | Ic | 5-CN | H | | 2′-Me, 5′-NO₂ | | CO | C₁₈H₁₄N₆O₃ |
| E84 | Ic | 5-CN | H | | 2′-Me, 5′-NO₂ | | CH₂ | C₁₈H₁₆N₆O₂ |
| E85 | Id | H | H | | 2′-Me, 5′-NO₂ | 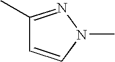 | SO₂ | C₁₇H₁₃N₅O₄S |
| E86 | Id | H | H | | 3′-NO₂ | 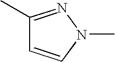 | SO₂ | C₁₆H₁₁N₅O₄S |
| E87 | Id | H | H | | 3′-CN | 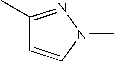 | SO₂ | C₁₇H₁₁N₅O₂S |
| E88 | Id | 5-Br | H | | 2′-Me, 5′-NO₂ | 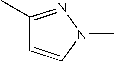 | SO₂ | C₁₇H₁₂BrN₅O₄S |
| E89 | Id | 5-Br | H | | 3′-NO₂ | 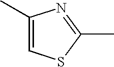 | SO | C₁₆H₉BrN₄O₃S₂ |
| E90 | Id | 5-Br | H | | 3′-NO₂ | 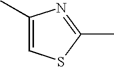 | SO₂ | C₁₆H₉BrN₄O₄S₂ |
| E91 | Id | 5-Br | H | | 2′-Me, 5′-NO₂ | 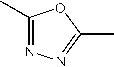 | S | C₁₆H₁₀BrN₅O₃S |
| E92 | Id | 5-Br | H | | 2′-Me, 5′-NO₂ | 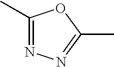 | SO | C₁₆H₁₀BrN₅O₄S |
| E93 | Id | 5-Br | H | | 2′-Me, 5′-Br | 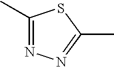 | SO₂ | C₁₆H₁₀Br₂N₄O₂S₂ |

Footnote for Table 1
^{a}All compounds were analysed by ¹H NMR and LCMS (APCl⁺), and had satisfactory combustion analyses for C, H, N.

The following examples are representative of the invention, and provide detailed methods for preparing the compounds of the invention including the preparation of intermediate compounds. In these examples, elemental analyses were carried out in the Microchemical Laboratory, University of Otago, Dunedin, NZ. Melting points were determined on an Electrothermal 2300 Melting Point Apparatus. ¹H NMR spectra were obtained on a Bruker Avance-400 spectrometer at 400 MHz, referenced to Me₄Si when measured in CDCl₃ and to the residual DMSO when measured in d₆-DMSO. Mass spectra were determined on a Thermo Finnigan MSQ single quadrupole mass spectrometer. Column chromatography was carried out on silica gel, (200-320 mesh, APS Finechem Ltd.) unless otherwise stated.

### Example 1

### N,2-Dimethyl-5-nitro-N′-(pyrazolo[1,5-a]pyridin-3-ylmethylene)benzene-sulfonohydrazide (E1)

Methylhydrazine sulfate (51 mg, 0.35 mmol) and NaHCO₃ (124 mg, 1.48 mmol) were added to a solution of pyrazolo[1,5-a]pyridine-3-carbaldehyde (13: X=Y=H) (43 mg, 0.29 mmol) [K. Tanji et al., Heterocycles 1993, 35 (2), 915] in MeOH (5 mL). After 1 h, 2-methyl-5-nitrobenzenesulfonyl chloride (104 mg, 0.44 mmol) was added and the reaction mixture stirred for a further 30 min. The solvent was removed in vacuo and the residue taken up in CH₂Cl₂ and water. The layers were separated and the aqueous phase extracted with CH₂Cl₂, then the combined organic layers were dried (Na₂SO₄) and the solvent removed in vacuo. Chromatography (eluting with hexanes:EtOAc 3:1 to 2:1 to 1:1) gave N,2-dimethyl-5-nitro-N′-(pyrazolo[1,5-a]pyridin-3-ylmethylene)benzenesulfonohydrazide (E1) as a yellow solid (63 mg, 57%). ¹H NMR δ (400 MHz, CDCl₃) 9.01 (d, J 2.4 Hz, 1H), 8.46 (d, J 7.0 Hz, 1H), 8.28 (dd, J 8.4, 2.4 Hz, 1H), 8.04 (s, 1H), 7.95 (s, 1H), 7.87 (d, J 8.9 Hz, 1H), 7.48 (d, J 8.4 Hz, 1H), 7.28 (m, 1H), 6.90 (td, J 7.0, 1.3 Hz, 1H), 3.41 (s, 3H), 2.76 (s, 3H). LCMS (APCI⁺) 374 (MH⁺, 100%). Anal. Calcd for C₁₆H₁₆N₆O₄S: C, 51.47; H, 4.05; N, 18.76. Found C, 51.72; H, 4.13; N, 18.98.

### Example 2

### N,2-Dimethyl-N′-((5-methylpyrazolo[1,5-a]pyridin-3-yl)methylene)-5-nitro-benzenesulfonohydrazide (E2)

Step 2.1: A fresh solution of O-(mesitylsulfonyl)hydroxylamine in CH₂Cl₂ (6.0 mL, 0.44 mol L⁻¹, 2.6 mmol) [T. Eichenberger et al., Helv. Chim. Acta 1986, 69 (6), 1521] was added to 4-methylpyridine (1: X=Me) (248 mg, 2.66 mmol) in CH₂Cl₂ (10 mL) at 0° C. After 2 h, the solvent was removed in vacuo. The residue was taken up in dry DMF (8 mL), then ethyl propiolate (0.25 mL, 2.5 mmol) and K₂CO₃ (450 mg, 3.26 mmol) were added, and the suspension stirred at room temperature for 18 h. The reaction mixture was diluted with water and extracted twice with EtOAc. The combined organic phases were washed three times with water then with brine, dried (Na₂SO₄) and the solvent removed in vacuo. Chromatography (eluting with hexanes:EtOAc 97:3 to 95:5 to 9:1) gave ethyl 5-methyl-pyrazolo[1,5-a]pyridine-3-carboxylate (4: X=Me) as a pale brown solid (175 mg, 53%). ¹H NMR δ (400 MHz, CDCl₃) 8.39 (d, J 7.1 Hz, 1H), 8.34 (s, 1H), 7.93 (s, 1H), 6.76 (dd, J 7.1, 1.9 Hz, 1H), 4.38 (q, J 7.1 Hz, 2H), 2.47 (s, 3H), 1.41 (t, J 7.1 Hz, 3H). LCMS (APCI⁺) 205 (MH⁺, 100%).

Step 2.2: A solution of 4 (X=Me) (173 mg, 0.85 mmol) in 1M NaOH (2.5 mL) and EtOH (5 mL) was stirred at room temperature for 48 h then refluxed for 3 h. The EtOH was removed in vacuo, and then the aqueous residue acidified to pH 1 with 1M HCl. The precipitated solid was filtered, washed with water and dried to leave 5-methylpyrazolo[1,5-a]pyridine-3-carboxylic acid (5: X=Me) as a white solid (136 mg, 91%). ¹H NMR δ (400 MHz, d₆-DMSO) 12.3 (br s, 1H), 8.70 (d, J 7.0 Hz, 1H), 8.30 (s, 1H), 7.86 (s, 1H), 6.96 (dd, J 7.0, 1.7 Hz, 1H), 2.44 (s, 3H).

Step 2.3: 1,1″-Carbonyldiimidazole (185 mg, 1.14 mmol) was added to a suspension of 5 (X=Me) (134 mg, 0.76 mmol) in dry THF (10 mL) under an atmosphere of N₂. After stirring for 18 h, the resulting solution was added dropwise to a solution of NaBH₄ (144 mg, 3.8 mmol) in H₂O (10 mL) and stirred for 30 min. The reaction was then quenched by the addition of 1M HCl and stirred for a further 30 min. The solution was neutralised with saturated aqueous NaHCO₃ and extracted twice with CH₂Cl₂. The combined organic layers were dried (Na₂SO₄) and the solvent removed in vacuo. Chromatography (eluting with hexanes:EtOAc 2:1 to 1:1 to EtOAc) gave (5-methylpyrazolo[1,5-a]pyridin-3-yl)methanol (6: X=Me) as a colourless oil (18 mg, 15%). ¹H NMR δ (400 MHz, CDCl₃) 8.33 (d, J 7.1 Hz, 1H), 7.89 (s, 1H), 7.38 (s, 1H), 6.60 (dd, J 7.1, 1.8 Hz, 1H), 4.83 (d, J=5.4 Hz, 2H), 2.36 (s, 3H), 1.41 (t, J 5.4 Hz, 1H). LCMS (APCI⁺) 163 (MH⁺, 100%).

Step 2.4: A suspension of 6 (X=Me) (18 mg, 0.11 mmol) and MnO₂ (97 mg, 1.1 mmol) in CH₂Cl₂ (2 mL) was stirred at room temperature for 4 days. The reaction mixture was then filtered through celite, washed with CH₂Cl₂, and the solvent removed from the filtrate in vacuo to leave 5-methylpyrazolo[1,5-a]pyridine-3-carbaldehyde (7: X=Me) as a white solid (14 mg, 78%). ¹H NMR δ (400 MHz, CDCl₃) 10.00 (s, 1H), 8.44 (d, J 7.0 Hz, 1H), 8.32 (s, 1H), 8.09 (s, 1H), 6.89 (dd, J 7.0, 1.7 Hz, 1H), 2.50 (s, 3H). LCMS (APCI⁺) 161 (MH⁺, 100%).

Step 2.5: Reaction of 7 (X=Me) (14 mg, 0.09 mmol) using the conditions of Example 1 gave N,2-dimethyl-N′-((5-methylpyrazolo[1,5-a]pyridin-3-yl)methylene)-5-nitrobenzene-sulfonohydrazide (E2) as a yellow solid (14 mg, 41%). ¹H NMR δ (400 MHz, CDCl₃) 9.00 (d, J 2.4 Hz, 1H), 8.34 (d, J 7.1 Hz, 1H), 8.30 (dd, J 8.4, 2.4 Hz, 1H), 8.00 (s, 1H), 7.99 (s, 1H), 7.61 (s, 1H), 7.49 (d, J 8.4 Hz, 1H), 6.72 (dd, J 7.1, 1.9 Hz, 1H), 3.39 (s, 3H), 2.76 (s, 3H), 2.38 (s, 3H). LCMS (APCI⁺) 388 (MH⁺, 100%). Anal. Calcd for C₁₇H₁₇N₅O₄S.0.1 EtOAc: C, 52.75; H, 4.53; N, 17.67. Found C, 52.66; H, 4.48; N, 17.49.

### Example 3

### N,2-Dimethyl-5-nitro-N′-((5-(trifluoromethyl)pyrazolo[1,5-a]pyridin-3-yl)-methylene)benzenesulfonohydrazide (E3)

Step 3.1: Reaction of 4-(trifluoromethyl)pyridine (1: X=CF₃) (441 mg, 3.00 mmol) using the conditions of Step 2.1 gave ethyl 5-(trifluoromethyl)pyrazolo[1,5-a]pyridine-3-carboxylate (8) as an off-white solid (276 mg, 36%). ¹H NMR δ (400 MHz, CDCl₃) 8.62 (d, J 7.3 Hz, 1H), 8.47-8.50 (m, 2H), 7.11 (dd, J 7.3, 2.0 Hz, 1H), 4.42 (q, J 7.1 Hz, 2H), 1.43 (t, J 7.1 Hz, 3H). LCMS (APCI⁺) 259 (MH⁺, 100%).

Step 3.2: LiAlH₄ (4.3 mL, 1.0 mol L⁻¹ in THF, 4.3 mmol) was added to a solution of 8 (276 mg, 1.07 mmol) in dry THF (10 mL) at 0° C. under an atmosphere of N₂. The reaction mixture was then warmed to room temperature and stirred for 1 h. The reaction was quenched by the dropwise addition of water, then filtered through a plug of celite and washed with CH₂Cl₂. The solvent was removed from the filtrate in vacuo. Chromatography (eluting with hexanes:EtOAc 2:1 to 1:1) gave (5-(trifluoro-methyl)pyrazolo[1,5-a]pyridin-3-yl)methanol (9) as a white solid (28 mg, 12%). ¹H NMR δ (400 MHz, CDCl₃) 8.54 (d, J 7.3 Hz, 1H), 8.05 (s, 1H), 8.00 (d, J 1.9 Hz, 1H), 6.94 (dd, J 7.3, 1.9 Hz, 1H), 4.93 (d, J 4.2 Hz, 2H), 3.48 (br s, 1H). LCMS (APCI⁺) 217 (MH⁺, 100%).

Step 3.3: Reaction of 9 (28 mg, 0.13 mmol) using the conditions of Step 2.4 gave 5-(trifluoromethyl)pyrazolo[1,5-a]pyridine-3-carbaldehyde (10) as an off-white solid (25 mg, 89%). ¹H NMR δ (400 MHz, CDCl₃) 10.12 (s, 1H), 8.69 (d, J 7.2 Hz, 1H), 8.63 (m, 1H), 8.50 (s, 1H), 7.24 (dd, J 7.2, 2.0 Hz, 1H). LCMS (APCI⁺) 215 (MH⁺, 100%).

Step 3.4: Reaction of 10 (25 mg, 0.12 mmol) using the conditions of Example 1 gave N,2-dimethyl-5-nitro-N′-((5-(trifluoromethyl)pyrazolo[1,5-a]pyridin-3-yl)methylene)benzene-sulfonohydrazide (E3) as a yellow solid (45 mg, 87%). ¹H NMR δ (400 MHz, CDCl₃) 8.92 (d, J 2.4 Hz, 1H), 8.55 (d, J 7.3 Hz, 1H), 8.32 (dd, J 8.4, 2.4 Hz, 1H), 8.17 (s, 1H), 8.10 (m, 1H), 7.94 (s, 1H), 7.50 (d, J 8.4 Hz, 1H), 7.03 (dd, J 7.3, 2.0 Hz, 1H), 3.45 (s, 3H), 2.76 (s, 3H). LCMS (APCI⁺) 442 (MH⁺, 100%). Anal. Calcd for C₁₇H₁₄F₃N₅O₄S.0.1 hexanes: C, 46.98; H, 3.45; N, 15.56. Found C, 46.99; H, 3.35; N, 15.84.

### Example 4

### N′-((2,5-Dimethylpyrazolo[1,5-a]pyridin-3-yl)methylene)-N,2-dimethyl-5-nitrobenzenesulfonohydrazide (E4)

Step 4.1: Reaction of 4-methylpyridine (1: X=Me) (250 mg, 2.68 mmol) and ethyl 2-butynoate (0.63 mL, 5.4 mmol) using the conditions of Step 6.1 gave ethyl 2,5-dimethyl-pyrazolo[1,5-a]pyridine-3-carboxylate (11: X=Y=Me) as a pale yellow solid (250 mg, 43%). ¹H NMR δ (400 MHz, CDCl₃) 8.27 (d, J 7.0 Hz, 1H), 7.86 (s, 1H), 6.69 (dd, J 7.0, 1.9 Hz, 1H), 4.38 (q, J 7.1 Hz, 2H), 2.65 (s, 3H), 2.44 (s, 3H), 1.42 (t, J 7.1 Hz, 3H). LCMS (APCI⁺) 219 (MH⁺, 100%).

Step 4.2: A solution of 11 (X=Y=Me) (60 mg, 0.28 mmol) in 40% aqueous H₂SO₄ (3 mL) was refluxed for 3 h. The solution was then cooled in ice and neutralised to pH 7 with 6M NaOH, then extracted twice with CH₂Cl₂. The combined extracts were dried (Na₂SO₄) and the solvent removed in vacuo to leave 2,5-dimethylpyrazolo[1,5-a]pyridine (12: X=Y=Me) as a pale yellow oil (32 mg, 80%). ¹H NMR δ (400 MHz, CDCl₃) 8.22 (d, J 7.1 Hz, 1H), 7.14 (s, 1H), 6.46 (dd, J 7.1, 1.8 Hz, 1H), 6.13 (s, 1H), 2.45 (s, 3H), 2.33 (s, 3H). LCMS (APCI⁺) 147 (MH⁺, 100%).

Step 4.3: A solution of 12 (X=Y=Me) (78 mg, 0.53 mmol) in dry DMF (2 mL) was treated with POCl₃ (0.15 mL, 1.6 mmol) at 0° C. under an atmosphere of N₂. The reaction mixture was then warmed to room temperature and stirred for 2 h. The solution was poured onto ice, basified to pH 10 with 1M NaOH, stirred for 1 h then extracted twice with CH₂Cl₂. The combined extracts were washed twice with water, dried (Na₂SO₄) and the solvent removed in vacuo to leave 2,5-dimethylpyrazolo[1,5-a]pyridine-3-carbaldehyde (13: X=Y=Me) as an off-white solid (77 mg, 83%). ¹H NMR δ (400 MHz, CDCl₃) 10.04 (s, 1H), 8.33 (d, J 7.0 Hz, 1H), 8.02 (s, 1H), 6.82 (dd, J 7.0, 1.9 Hz, 1H), 2.66 (s, 3H), 2.47 (s, 3H). LCMS (APCI⁺) 175 (MH⁺, 100%).

Step 4.4: Reaction of 13 (X=Y=Me) (38 mg, 0.22 mmol) using the conditions of Example 1 gave N′-((2,5-dimethylpyrazolo[1,5-a]pyridin-3-yl)methylene)-N,2-dimethyl-5-nitrobenzenesulfonohydrazide as a yellow solid (E4) (22 mg, 25%). ¹H NMR δ (400 MHz, CDCl₃) 8.99 (d, J 2.4 Hz, 1H), 8.30 (dd, J 8.4, 2.4 Hz, 1H), 8.23 (d, J 7.0 Hz, 1H), 8.04 (s, 1H), 7.57-7.44 (m, 2H), 6.65 (dd, J 7.0, 1.9 Hz, 1H), 3.38 (s, 3H), 2.75 (s, 3H), 2.47 (s, 3H), 2.35 (s, 3H). LCMS (APCI⁺) 402 (MH⁺, 100%). Anal. Calcd for C₁₈H₁₉N₅O₄S.0.2 EtOAc: C, 53.88; H, 4.95; N, 16.71. Found C, 53.86; H, 5.11; N, 16.86.

### Example 5

### N′-((5-Cyanopyrazolo[1,5-a]pyridin-3-yl)methylene)-N,2-dimethyl-5-nitro-benzenesulfonohydrazide (E5)

Step 5.1: Reaction of methyl isonicotinate (1: X=CO₂Me) (2.45 g, 17.9 mmol) using the conditions of Step 2.1 gave 3-ethyl 5-methylpyrazolo[1,5-a]pyridine-3,5-dicarboxylate (14) as a pale brown solid (1.88 g, 42%). ¹H NMR δ (400 MHz, CDCl₃) 8.86 (dd, J 1.8, 0.9 Hz, 1H), 8.55 (dd, J 7.2, 0.9 Hz, 1H), 8.47 (s, 1H), 7.52 (dd, J 7.2, 1.8 Hz, 1H), 4.42 (q, J 7.1 Hz, 2H), 3.99 (s, 3H), 1.44 (t, J 7.1 Hz, 3H). LCMS (APCI⁺) 249 (MH⁺, 100%).

Step 5.2: A solution of 14 (2.65 g, 10.7 mmol) in 40% aqueous H₂SO₄ (50 mL) was refluxed for 16 h. The solution was then cooled in ice and basified to pH 2 with 6M NaOH. The precipitated solid was filtered off, washed with water and dried to leave pyrazolo[1,5-a]pyridine-5-carboxylic acid (15) as an off-white solid (1.60 g, 92%). ¹H NMR δ (400 MHz, d₆-DMSO) 12.5 (br s, 1H), 8.74 (d, J 7.3 Hz, 1H), 8.34 (m, 1H), 8.11 (d, J 2.3 Hz, 1H), 7.25 (dd, J 7.3, 1.9 Hz, 1H), 6.89 (dd, J 2.3, 0.8 Hz, 1H). LCMS (APCI⁻) 161 (M-H, 100%).

Step 5.3: 1,1″-Carbonyldiimidazole (2.40 g, 14.8 mmol) was added to a suspension of 15 (1.60 g, 9.9 mmol) in dry THF (50 mL) under an atmosphere of N₂ and stirred for 18 h. The solution was then poured into concentrated NH₃ (30 mL) at 0° C. and stirred for 2 h. After 2 h, the solvent was removed in vacuo. The crude pyrazolo[1,5-a]pyridine-5-carboxamide (16) was taken up in dry DMF (50 mL) and cooled to 0° C., then POCl₃ (17.5 mL, 0.19 mol) was added. After 30 min the ice bath was removed and the reaction stirred for a further 18 h at room temperature. The solution was then poured onto ice, basified to pH 10 with 6M NaOH, stirred for 1 h and extracted twice with CH₂Cl₂. The combined extracts were washed twice with water, dried (Na₂SO₄) and the solvent removed in vacuo. Chromatography (eluting with hexanes:EtOAc 3:1 to 2:1 to 1:1) gave 3-formylpyrazolo-[1,5-a]pyridine-5-carbonitrile (17) as a pale yellow solid (927 mg, 55%). ¹H NMR δ (400 MHz, CDCl₃) 10.12 (s, 1H), 8.70 (m, 1H), 8.66 (d, J 7.1 Hz, 1H), 8.52 (s, 1H), 7.19 (dd, J 7.1, 1.7 Hz, 1H). LCMS (APCI⁺) 172 (MH⁺, 100%).

Step 5.4: Reaction of 17 (49 mg, 0.29 mmol) using the conditions of Example 1 gave N′-((5-cyanopyrazolo[1,5-a]pyridin-3-yl)methylene)-N,2-dimethyl-5-nitrobenzene-sulfonohydrazide (E5) as a yellow solid (59 mg, 52%). ¹H NMR δ (400 MHz, CDCl₃) 8.91 (d, J 2.4 Hz, 1H), 8.52 (d, J 7.2 Hz, 1H), 8.37 (dd, J 8.4, 2.4 Hz, 1H), 8.18 (s, 1H), 8.04 (m, 1H), 7.90 (s, 1H), 7.57 (d, J 8.4 Hz, 1H), 6.98 (dd, J 7.2, 1.7 Hz, 1H), 3.45 (s, 3H), 2.79 (s, 3H). LCMS (APCI⁺) 399 (MH⁺, 100%). Anal. Calcd for C₁₇H₁₄N₆O₄S: C, 51.25; H, 3.54; N, 21.09. Found C, 50.95; H, 3.54; N, 20.90.

### Example 6

### Methyl 3-((2-methyl-2-(2-methyl-5-nitrophenylsulfonyl)hydrazono)methyl)pyrazolo[1,5-a]pyridine-5-carboxylate (E6)

Step 6.1: A solution of pyrazolo[1,5-a]pyridine-5-carboxylic acid (15) (31 mg, 0.19 mmol) and concentrated HCl (3 drops) in MeOH (10 mL) was refluxed for 4 h. The solvent was removed in vacuo, saturated aqueous NaHCO₃ was added to the residue, and then it was extracted twice with CH₂Cl₂. The combined extracts were dried (Na₂SO₄) and the solvent removed in vacuo to leave methylpyrazolo[1,5-a]pyridine-5-carboxylate (18) as an off-white solid (25 mg, 74%). ¹H NMR δ (400 MHz, CDCl₃) 8.52 (dt, J 7.3, 0.9 Hz, 1H), 8.33 (dd, J 1.8, 0.9 Hz, 1H), 8.04 (d, J 2.3 Hz, 1H), 7.34 (dd, J 7.3, 1.8 Hz, 1H), 6.75 (dd, J 2.3, 0.9 Hz, 1H), 3.96 (s, 3H). LCMS (APCI⁺) 177 (MH⁺, 100%).

Step 6.2: Reaction of 18 (25 mg, 0.14 mmol) using the conditions of Step 4.3 gave methyl 3-formylpyrazolo[1,5-a]pyridine-5-carboxylate (19) as a yellow solid (22 mg, 76%). ¹H NMR δ (400 MHz, CDCl₃) 10.12 (s, 1H), 8.95 (dd, J 1.8, 0.9 Hz, 1H), 8.61 (dd, J 7.2, 0.9 Hz, 1H), 8.47 (s, 1H), 7.64 (dd, J 7.2, 1.8 Hz, 1H), 4.01 (s, 3H). LCMS (APCI⁺) 205 (MH⁺, 100%).

Step 6.3: Reaction of 19 (22 mg, 0.11 mmol) using the conditions of Example 1 gave methyl 3-((2-methyl-2-(2-methyl-5-nitrophenylsulfonyl)hydrazono)methyl)pyrazolo[1,5-a]pyridine-5-carboxylate (E6) as a yellow solid (43 mg, 93%). ¹H NMR δ (400 MHz, CDCl₃) 8.95 (d, J 2.4 Hz, 1H), 8.51 (dd, J 1.9, 0.9 Hz, 1H), 8.47 (dd, J 7.2, 0.9 Hz, 1H), 8.32 (dd, J 8.4, 2.4 Hz, 1H), 8.13 (s, 1H), 7.96 (s, 1H), 7.50 (d, J 8.4 Hz, 1H), 7.42 (dd, J 7.2, 1.9 Hz, 1H), 3.98 (s, 3H), 3.44 (s, 3H), 2.78 (s, 3H). LCMS (APCI⁺) 432 (MH⁺, 100%). Anal. Calcd for C₁₈H₁₇N₆O₆S.0.2H₂O: C, 49.70; H, 4.03; N, 16.10. Found C, 49.65; H, 4.08; N, 15.79.

### Example 7

### 3-((2-Methyl-2-(2-methyl-5-nitrophenylsulfonyl)hydrazono)methyl)-pyrazolo[1,5-a]pyridine-5-carboxamide (E7)

Step 7.1: A solution of methyl 3-formylpyrazolo[1,5-a]pyridine-5-carboxylate (19) (1.23 g, 6.0 mmol) in 1M NaOH (18 mL) and EtOH (40 mL) was stirred at room temperature for 18 h. The EtOH was removed in vacuo, the aqueous residue acidified to pH 1 with 1M HCl. The precipitated product was filtered and washed with water to leave 3-formylpyrazolo[1,5-a]pyridine-5-carboxylic acid (20) as a white solid (0.98 g, 85%). ¹H NMR δ (400 MHz, d₆-DMSO) 13.8 (br s, 1H), 10.10 (s, 1H), 9.01 (dd, J 7.1, 0.8 Hz, 1H), 8.76 (s, 1H), 8.74 (m, 1H), 7.58 (dd, J 7.1, 1.9 Hz, 1H). LCMS (APCI⁺) 191 (MH⁺, 100%).

Step 7.2: A solution of 20 (60 mg, 0.32 mmol) in SOCl₂ (1 mL) was refluxed for 1 h. The solvent was removed in vacuo, and then the residue was taken up in CH₂Cl₂ (5 mL) and added to concentrated NH₃ (5 mL). After 30 min the reaction mixture was acidified to pH 1 with 1M HCl, saturated with NaCl and extracted four times with CH₂Cl₂. The combined extracts were dried (Na₂SO₄) and the solvent removed in vacuo to leave 3-formylpyrazolo-[1,5-a]pyridine-5-carboxamide (21) as a white solid (29 mg, 48%). ¹H NMR δ (400 MHz, d₆-DMSO) 10.09 (s, 1H), 9.04 (dd, J 7.2, 0.9 Hz, 1H), 8.75 (s, 1H), 8.73 (dd, J 1.9, 0.9 Hz, 1H), 8.41 (br s, 1H), 7.77 (br s, 1H), 7.62 (dd, J 7.2, 1.9 Hz, 1H). LCMS (APCI⁻) 188 (M-H, 100%).

Step 7.3: Methylhydrazine sulfate (43 mg, 0.30 mmol) and NaHCO₃ (100 mg, 1.19 mmol) were added to a suspension of 21 (28 mg, 0.15 mmol) in MeOH (5 mL). After 2 h, 2-methyl-5-nitrobenzenesulfonyl chloride (70 mg, 0.30 mmol) was added and the reaction mixture stirred for a further 30 min. The solvent was removed in vacuo and the residue taken up in CH₂Cl₂ and water. The solid was filtered off, washed with water and CH₂Cl₂ then dried to leave 3-((2-methyl-2-(2-methyl-5-nitrophenylsulfonyl)-hydrazono)methyl)pyrazolo[1,5-a]pyridine-5-carboxamide (E7) as a yellow solid (30 mg, 48%). ¹H NMR δ (400 MHz, d₆-DMSO) 8.84 (dd, J 7.3, 0.8 Hz, 1H), 8.68 (d, J 2.5 Hz, 1H), 8.40 (dd, J 8.4, 2.5 Hz, 1H), 8.34 (dd, J 1.9, 0.8 Hz, 1H), 8.31 (s, 1H), 8.24 (s, 1H), 8.07 (br s, 1H), 7.74 (d, J 8.4 Hz, 1H), 7.58 (br s, 1H), 7.40 (dd, J 7.3, 1.9 Hz, 1H), 3.39 (s, 3H), 2.74 (s, 3H). LCMS (APCI⁺) 417 (MH⁺, 100%). Anal. Calcd for C₁₇H₁₆N₆O₆S: C, 49.03; H, 3.87; N, 20.18. Found C, 49.29; H, 3.92; N, 20.30.

### Example 8

### N′-((5-(2-Hydroxyethyl)pyrazolo[1,5-a]pyridin-3-yl)methylene)-N,2-dimethyl-5-nitrobenzenesulfonohydrazide (E8)

Step 8.1: Reaction of 4-(2-hydroxyethyl)pyridine (1: X=CH₂CH₂OH) (308 mg, 2.50 mmol) using the conditions of Step 2.1 gave ethyl 5-(2-hydroxyethyl)pyrazolo[1,5-a]pyridine-3-carboxylate (22) as a pale brown solid (141 mg, 24%). ¹H NMR δ (400 MHz, CDCl₃) 8.44 (d, J 7.1 Hz, 1H), 8.36 (s, 1H), 8.02 (s, 1H), 6.86 (dd, J 7.1, 1.9 Hz, 1H), 4.38 (q, J 7.1 Hz, 2H), 4.18 (br s, 1H), 3.98 (q, J 6.4 Hz, 2H), 2.98 (t, J 6.4 Hz, 2H), 1.41 (t, J 7.1 Hz, 3H). LCMS (APCI⁺) 235 (MH⁺, 100%).

Step 8.2: Reaction of 22 (141 mg, 0.60 mmol) using the conditions of Step 4.2 gave 2-(pyrazolo[1,5-a]pyridin-5-yl)ethanol (23) as a yellow oil (48 mg, 49%). ¹H NMR δ (400 MHz, CDCl₃) 8.40 (d, J 7.2 Hz, 1H), 7.91 (d, J 2.2 Hz, 1H), 7.38 (m, 1H), 6.64 (dd, J 7.2, 1.9 Hz, 1H), 6.42 (dd, J 2.2, 0.7 Hz, 1H), 3.93 (t, J 6.4 Hz, 2H), 2.89 (t, J 6.4 Hz, 2H). LCMS (APCI⁺) 163 (MH⁺, 100%).

Step 8.3: A solution of 23 (48 mg, 0.30 mmol), Ac₂O (84 μL, 0.90 mmol) and pyridine (96 μL, 1.2 mmol) in CH₂Cl₂ (5 mL) was stirred at room temperature for 18 h. The reaction mixture was diluted with CH₂Cl₂, washed with saturated aqueous NaHCO₃, dried (Na₂SO₄) and the solvent removed in vacuo to leave 2-(pyrazolo[1,5-a]pyridin-5-yl)ethyl acetate (24) as a yellow oil (60 mg, 100%). ¹H NMR δ (400 MHz, CDCl₃) 8.40 (d, J 7.2 Hz, 1H), 7.92 (d, J 2.3 Hz, 1H), 7.35 (m, 1H), 6.62 (dd, J 7.2, 1.9 Hz, 1H), 6.43 (dd, J 2.3, 0.8 Hz, 1H), 4.33 (t, J 6.8 Hz, 2H), 2.96 (t, J 6.8 Hz, 2H), 2.04 (s, 3H). LCMS (APCI⁺) 205 (MH⁺, 100%).

Step 8.4: Reaction of 24 (60 mg, 0.29 mmol) using the conditions of Step 4.3 gave 2-(3-formylpyrazolo[1,5-a]pyridin-5-yl)ethyl acetate (25) as a yellow oil (48 mg, 71%). ¹H NMR δ (400 MHz, CDCl₃) 10.02 (s, 1H), 8.50 (d, J 7.1 Hz, 1H), 8.36 (s, 1H), 8.16 (m, 1H), 6.96 (dd, J 7.1, 1.9 Hz, 1H), 4.38 (t, J 6.6 Hz, 2H), 3.07 (t, J 6.6 Hz, 2H), 2.05 (s, 3H). LCMS (APCI⁺) 233 (MH⁺, 100%).

Step 8.5: A solution of 25 (28 mg, 0.12 mmol) in 1M NaOH (0.36 mL) and EtOH (2 mL) was refluxed for 17 h. The EtOH was removed in vacuo and the residue extracted twice with CH₂Cl₂. The combined extracts were dried (Na₂SO₄) and the solvent removed in vacuo to leave 5-(2-hydroxyethyl)pyrazolo[1,5-a]pyridine-3-carbaldehyde (26) as a yellow solid (18 mg, 78%). ¹H NMR δ (400 MHz, CDCl₃) 10.01 (s, 1H), 8.50 (d, J 7.1 Hz, 1H), 8.35 (s, 1H), 8.17 (m, 1H), 7.00 (dd, J 7.1, 1.8 Hz, 1H), 3.99 (m, 2H), 3.01 (t, J 6.3 Hz, 2H), 1.47 (t, J 5.4 Hz, 1H). LCMS (APCI⁺) 191 (MH⁺, 100%).

Step 8.6: Reaction of 26 (18 mg, 0.10 mmol) using the conditions of Example 1 gave N-((5-(2-hydroxyethyl)pyrazolo[1,5-a]pyridin-3-yl)methylene)-N,2-dimethyl-5-nitrobenzenesulfonohydrazide (E8) as a yellow solid (20 mg, 50%). ¹H NMR δ (400 MHz, CDCl₃) 9.03 (d, J 2.4 Hz, 1H), 8.39 (dd, J 7.1, 0.8 Hz, 1H), 8.30 (dd, J 8.4, 2.4 Hz, 1H), 8.02 (s, 1H), 7.96 (s, 1H), 7.69 (m, 1H), 7.50 (d, J 8.4 Hz, 1H), 6.81 (dd, J 7.1, 1.9 Hz, 1H), 3.88 (m, 2H), 3.42 (s, 3H), 2.87 (t, J 6.4 Hz, 2H), 2.75 (s, 3H), 1.54 (br s, 1H). LCMS (APCI⁺) 418 (MH⁺, 100%). Anal. Calcd for C₁₈H₁₉N₅O₅S.0.33 EtOAc: C, 51.97; H, 4.88; N, 15.68. Found C, 51.58; H, 4.74; N, 15.51.

### Example 9

### N′-((5-Methoxypyrazolo[1,5-a]pyridin-3-yl)methylene)-N,2-dimethyl-5-nitrobenzenesulfonohydrazide (E9)

Step 9.1: A solution of O-(2,4-dinitrophenyl)hydroxylamine (741 mg, 2.2 mmol) [C. Legault et al., J. Org. Chem. 2003, 68 (18), 7119] and 4-methoxypyridine (1: X=OMe) (244 mg, 2.24 mmol) were heated at 40° C. for 18 h. The solvent was removed in vacuo, then the residue was taken up in dry DMF (10 mL). Ethyl propiolate (0.27 mL, 2.7 mmol) and K₂CO₃ (6180 mg, 4.47 mmol) were added, and the suspension stirred at room temperature for 24 h. The reaction mixture was diluted with water and extracted twice with EtOAc. The combined organic phases were washed three times with water then with brine, dried (Na₂SO₄) and the solvent removed in vacuo. Chromatography (eluting with hexanes:EtOAc 9:1 to 4:1) gave ethyl 5-methoxypyrazolo[1,5-a]pyridine-3-carboxylate (27) as a yellow solid (114 mg, 23%). ¹H NMR δ (400 MHz, CDCl₃) 8.31 (d, J 7.5 Hz, 1H), 8.28 (s, 1H), 7.43 (d, J 2.8 Hz, 1H), 6.61 (dd, J 7.5, 2.8 Hz, 1H), 4.37 (q, J 7.1 Hz, 2H), 3.93 (s, 3H), 1.41 (t, J 7.1 Hz, 3H). LCMS (APCI⁺) 221 (MH⁺, 100%).

Step 9.2: Reaction of 27 (114 mg, 0.52 mmol) using the conditions of Step 4.2 gave pyrazolo[1,5-a]pyridin-5-ol (28) as a pale brown solid (61 mg, 88%). ¹H NMR δ (400 MHz, d₆-DMSO) 10.06 (s, 1H), 8.45 (d, J 7.5 Hz, 1H), 7.79 (d, J 2.2 Hz, 1H), 6.77 (d, J 2.4 Hz, 1H), 6.46 (dd, J 7.5, 2.4 Hz, 1H), 6.23 (d, J 2.2 Hz, 1H). LCMS (APCI⁺) 135 (MH⁺, 100%).

Step 9.3: Iodomethane (23 μL, 0.37 mmol) was added to a suspension of 28 (25 mg, 0.19 mmol) and K₂CO₃ (52 mg, 0.38 mmol) in DMF (2 mL). After 3 h, the reaction mixture was diluted with water and extracted twice with CH₂Cl₂. The combined extracts were dried (Na₂SO₄) and the solvent removed in vacuo to leave 5-methoxypyrazolo[1,5-a]pyridine (29) as a pale brown oil (24 mg, 86%). ¹H NMR δ (400 MHz, CDCl₃) 8.28 (d, J 7.6 Hz, 1H), 7.85 (d, J 2.2 Hz, 1H), 6.74 (d, J 2.7 Hz, 1H), 6.44 (dd, J 7.6, 2.7 Hz, 1H), 6.32 (d, J 2.2 Hz, 1H), 3.84 (s, 3H). LCMS (APCI⁺) 149 (MH⁺, 100%).

Step 9.4: Reaction of 29 (24 mg, 0.16 mmol) using the conditions of Step 4.3 gave 5-methoxypyrazolo[1,5-a]pyridine-3-carbaldehyde (13: X=OMe, Y=H) [J. Elsner et al., Bioorg. Med. Chem. 2006, 14 (6), 1949] as a pale yellow solid (27 mg, 93%). ¹H NMR δ (400 MHz, CDCl₃) 9.96 (s, 1H), 8.37 (d, J 7.5 Hz, 1H), 8.27 (s, 1H), 7.60 (d, J 2.7 Hz, 1H), 6.72 (dd, J 7.5, 2.7 Hz, 1H), 3.96 (s, 3H). LCMS (APCI⁺) 177 (MH⁺, 100%).

Step 9.5: A solution of 13 (X=OMe, Y=H) (27 mg, 0.15 mmol) and 2-methyl-5-nitro-benzenesulfonohydrazide (45) (39 mg, 0.17 mmol) [I. Kh. Fel'dman et al., Zh. Obshch. Khim., 1963, 33, 38] in MeOH (5 mL) was stirred at room temperature for 4 h. THF (2 mL) was then added, followed by the dropwise addition of CH₂N₂ solution in Et₂O until gas evolution ceased. The solvents were removed in vacuo. Chromatography (eluting with CH₂Cl₂: MeOH 99.5:0.5) gave N′-((5-methoxypyrazolo[1,5-a]pyridin-3-yl)methylene)-N,2-dimethyl-5-nitrobenzenesulfonohydrazide (E9) as a yellow solid (36 mg, 58%). ¹H NMR δ (400 MHz, d₆-DMSO) 8.67 (d, J 2.5 Hz, 1H), 8.62 (d, J 7.6 Hz, 1H), 8.39 (dd, J 8.4, 2.5 Hz, 1H), 8.15 (s, 1H), 8.12 (s, 1H), 7.74 (d, J 8.4 Hz, 1H), 7.26 (d, J 2.8 Hz, 1H), 6.74 (dd, J 7.6, 2.8 Hz, 1H), 3.81 (s, 3H), 3.29 (s, 3H), 2.71 (s, 3H). LCMS (APCI⁺) 404 (MH⁺, 100%). Anal. Calcd for C₁₇H₁₇N₆O₆S: C, 50.61; H, 4.25; N, 17.36. Found C, 50.85; H, 4.29; N, 17.51.

### Example 10

### 3-((2-Methyl-2-(2-methyl-5-nitrophenylsulfonyl)hydrazono)methyl)-pyrazolo[1,5-a]pyridin-5-yl acetate (E10)

Step 10.1: Reaction of pyrazolo[1,5-a]pyridin-5-ol (28) (36 mg, 0.27 mmol) using the conditions of Step 4.3 gave 5-hydroxypyrazolo[1,5-a]pyridine-3-carbaldehyde (30) as a red-brown solid (42 mg, 95%). ¹H NMR δ (400 MHz, d₆-DMSO) 11.10 (s, 1H), 9.83 (s, 1H), 8.71 (d, J 7.4 Hz, 1H), 8.44 (s, 1H), 7.42 (d, J 2.6 Hz, 1H), 6.77 (dd, J 7.4, 2.6 Hz, 1H). LCMS (APCI⁺) 163 (MH⁺, 100%).

Step 10.2: A solution of 30 (42 mg, 0.26 mmol), Ac₂O (37 μL, 0.39 mmol) and NEt₃ (54 μL, 0.39 mmol) in CH₂Cl₂ (10 mL) was stirred at room temperature for 3 days. The reaction mixture was diluted with CH₂Cl₂ and washed with water, dried (Na₂SO₄) and the solvent removed in vacuo. Chromatography (eluting with hexanes:EtOAc 3:1 to 2:1) gave 3-formylpyrazolo[1,5-a]pyridin-5-yl acetate (31) as a pale brown solid (48 mg, 91%). ¹H NMR δ (400 MHz, CDCl₃) 10.02 (s, 1H), 8.54 (dd, J 7.4 Hz, 1H), 8.38 (s, 1H), 8.05 (d, J 2.5 Hz, 1H), 6.91 (dd, J 7.4, 2.5 Hz, 1H), 2.37 (s, 3H). LCMS (APCI⁺) 205 (MH⁺, 100%).

Step 10.3: Reaction of 31 (48 mg, 0.24 mmol) using the conditions of Step 9.5 gave 3-((2-methyl-2-(2-methyl-5-nitrophenylsulfonyl)hydrazono)methyl)pyrazolo[1,5-a]pyridin-5-yl acetate (E10) as a yellow solid (72 mg, 71%). ¹H NMR δ (400 MHz, CDCl₃) 9.02 (d, J 2.4 Hz, 1H), 8.41 (dd, J 7.5, 0.7 Hz, 1H), 8.30 (dd, J 8.4, 2.4 Hz, 1H), 8.03 (s, 1H), 7.92 (s, 1H), 7.51 (d, J 2.5 Hz, 1H), 7.48 (d, J 8.4 Hz, 1H), 6.71 (dd, J 7.5, 2.5 Hz, 1H), 3.42 (s, 3H), 2.72 (s, 3H), 2.39 (s, 3H). LCMS (APCI⁺) 432 (MH⁺, 100%). Anal. Calcd for C₁₈H₁₇N₅O₆S.0.5H₂O: C, 49.09; H, 4.12; N, 15.90. Found C, 49.31; H, 4.00; N, 15.91.

### Example 11

### N′-((5-Hydroxypyrazolo[1,5-a]pyridin-3-yl)methylene)-N,2-dimethyl-5-nitrobenzenesulfonohydrazide (E11)

Saturated aqueous NaHCO₃ (5 mL) was added to a suspension of E10 (52 mg, 0.12 mmol) in MeOH (10 mL) and stirred for 2 h. The MeOH was removed in vacuo, and the resulting solid filtered off and washed with water. Chromatography (eluting with CH₂Cl₂: MeOH 99:1 to 19:1) gave N′-((5-hydroxypyrazolo[1,5-a]pyridin-3-yl)methylene)-N,2-dimethyl-5-nitrobenzenesulfonohydrazide (E11) as a yellow solid (26 mg, 55%). ¹H NMR δ (400 MHz, d₆-DMSO) 10.58 (br s, 1H), 8.69 (d, J 2.5 Hz, 1H), 8.54 (d, J 7.5 Hz, 1H), 8.41 (dd, J 8.4, 2.5 Hz, 1H), 8.12 (s, 1H), 8.06 (s, 1H), 7.74 (d, J 8.4 Hz, 1H), 6.88 (d, J 2.6 Hz, 1H), 6.60 (dd, J 7.5, 2.6 Hz, 1H), 3.27 (s, 3H), 2.69 (s, 3H). LCMS (APCI⁺) 390 (MH⁺, 100%). Anal. Calcd for C₁₆H₁₅N₅O₅S: C, 49.35; H, 3.88; N, 17.99. Found C, 49.51; H, 3.94; N, 17.72.

### Example 12

### N′-((5-Aminopyrazolo[1,5-a]pyridin-3-yl)methylene)-N,2-dimethyl-5-nitrobenzenesulfonohydrazide (E12)

Step 12.1: Reaction of tert-butyl pyridin-4-ylcarbamate (1: X=NHCO₂^{t}Bu) (1.36 g, 7.0 mmol) using the conditions of Step 9.1 gave ethyl 5-(tert-butoxycarbonylamino)pyrazolo-[1,5-a]pyridine-3-carboxylate (32) as a yellow solid (707 mg, 36%). ¹H NMR δ (400 MHz, CDCl₃) 8.38 (d, J 7.6 Hz, 1H), 8.32 (s, 1H), 7.95 (d, J 1.9 Hz, 1H), 7.28 (m, 1H), 6.71 (s, 1H), 4.37 (q, J 7.1 Hz, 2H), 1.55 (s, 9H), 1.41 (t, J 7.1 Hz, 3H). LCMS (APCI⁺) 306 (MH⁺, 100%).

Step 12.2: A solution of 32 (707 mg, 2.32 mmol) and trifluoroacetic acid (3.6 mL, 47 mmol) in CH₂Cl₂ (20 mL) was stirred at room temperature for 18 h. The solvents were removed in vacuo to leave the trifluoroacetate salt of ethyl 5-aminopyrazolo[1,5-a]pyridine-3-carboxylate (33) as a brown solid (1.01 g, 100%). ¹H NMR δ (400 MHz, CDCl₃) 8.37 (dd, J 7.4, 0.6 Hz, 1H), 8.25 (s, 1H), 7.22 (dd, J 2.6, 0.6 Hz, 1H), 6.40 (dd, J 7.4, 2.6 Hz, 1H), 4.35 (q, J 7.1 Hz, 2H), 1.39 (t, J 7.1 Hz, 3H). LCMS (APCI⁺) 206 (MH⁺, 100%).

Step 12.3: Reaction of 33 (1.29 g, 2.98 mmol) using the conditions of Step 4.2 except with carrying out the aqueous extraction from pH 12 gave pyrazolo[1,5-a]pyridin-5-amine (34) as a pale brown solid (310 mg, 78%). ¹H NMR δ (400 MHz, CDCl₃) 8.23 (d, J 7.4 HZ, 1H), 7.79 (d, J 2.0 Hz, 1H), 6.58 (d, J 2.4 Hz, 1H), 6.22 (dd, J 7.4, 2.4 Hz, 1H), 6.13 (d, J 2.0 Hz, 1H), 3.81 (s, 2H). LCMS (APCI⁺) 134 (MH⁺, 100%).

Step 12.4: Trifluoroacetic anhydride (0.43 mL, 3.0 mmol) was added dropwise to a solution of 34 (270 mg, 2.03 mmol) and NEt₃ (0.42 mL, 3.0 mmol) in CH₂Cl₂ (20 mL) at 0° C. over 5 min. After 1 h, the reaction mixture was washed with water, dried (Na₂SO₄) and the solvent removed in vacuo. Chromatography (eluting with hexanes:EtOAc 3:1) gave 2,2,2-trifluoro-N-(pyrazolo[1,5-a]pyridin-5-yl)acetamide (35) as a yellow solid (242 mg, 52%). ¹H NMR δ (400 MHz, CDCl₃) 8.46 (d, J 7.5 Hz, 1H), 8.04 (d, J 2.3 Hz, 1H), 7.98 (d, J 2.3 Hz, 1H), 7.87 (s, 1H), 6.78 (dd, J 7.5, 2.3 Hz, 1H), 6.54 (dd, J 2.3, 0.8 Hz, 1H). LCMS (APCI⁺) 230 (MH⁺, 100%).

Step 12.5: POCl₃ (0.30 mL, 3.2 mmol) was added to a solution of 35 (242 mg, 1.06 mmol) in dry DMF (5 mL) at 0° C. under an atmosphere of N₂. The reaction mixture was then warmed to room temperature and stirred for 2 h. The solution was poured onto ice, basified to pH 14 with 1M NaOH, diluted with MeOH (10 mL) and refluxed for 2 h. The MeOH was removed in vacuo, the resulting solution acidified to pH 10 with 1M HCl and extracted twice with CH₂Cl₂. The combined extracts were dried (Na₂SO₄) and the solvent removed in vacuo. Chromatography (eluting with hexanes:EtOAc 2:1 to 1:1 to EtOAc) gave 5-aminopyrazolo[1,5-a]pyridine-3-carbaldehyde (36) as a yellow solid (141 mg, 83%). ¹H NMR δ (400 MHz, d₆-DMSO) 9.69 (s, 1H), 8.43 (d, J 7.4 Hz, 1H), 8.25 (s, 1H), 7.10 (d, J 2.4 Hz, 1H), 6.56 (dd, J 7.4, 2.4 Hz, 1H), 6.48 (s, 2H). LCMS (APCI⁺) 162 (MH⁺, 100%).

Step 12.6: Trifluoroacetic anhydride (95 μL, 0.67 mmol) was added to a solution of 36 (72 mg, 0.45 mmol) and NEt₃ (93 μL, 0.67 mmol) in CH₂Cl₂ (10 mL) at 0° C. After 1 h, the solvent was removed in vacuo and the residue triturated with water, filtered and dried to leave 2,2,2-trifluoro-N-(3-formylpyrazolo[1,5-a]pyridin-5-yl)acetamide (37) as an orange solid (83 mg, 72%). ¹H NMR δ (400 MHz, d₆-DMSO) 11.81 (br s, 1H), 9.95 (s, 1H), 8.92 (d, J 7.2 Hz, 1H), 8.67 (s, 1H), 8.62 (s, 1H), 7.49 (d, J 7.2 Hz, 1H). LCMS (APCI⁺) 258 (MH⁺, 100%).

Step 12.7: Reaction of 37 (82 mg, 0.32 mmol) using the conditions of Step 9.5 gave 2,2,2-trifluoro-N-(3-((2-methyl-2-(2-methyl-5-nitrophenylsulfonyl)hydrazono)methyl)pyrazolo[1,5-a]pyridin-5-yl)acetamide (50) as a yellow solid (119 mg, 77%). ¹H NMR δ (400 MHz, d₆-DMSO) 11.60 (s, 1H), 8.73-8.78 (m, 2H), 8.49 (d, J 2.3 Hz, 1H), 8.33 (dd, J 8.4, 2.5 Hz, 1H), 8.24 (s, 1H), 8.18 (s, 1H), 7.70 (d, J 8.4 Hz, 1H), 7.19 (dd, J 7.5, 2.3 Hz, 1H), 3.35 (s, 3H), 2.69 (s, 3H). LCMS (APCI⁺) 485 (MH⁺, 100%). Anal. Calcd for C₁₈H₁₆F₃N₆O₆S.0.05 PhMe: C, 45.07; H, 3.17; N, 17.19. Found C, 44.77; H, 3.36; N, 16.84.

Step 12.8: Na₂CO₃ (42 mg, 0.40 mmol) was added to a solution of 50 (95 mg, 0.20 mmol) in MeOH (10 mL) and water (5 mL) and stirred for 18 h. The precipitated solid was filtered off and washed with MeOH and water to leave N′-((5-aminopyrazolo[1,5-a]pyridin-3-yl)methylene)-N,2-dimethyl-5-nitrobenzenesulfonohydrazide (E12) as a yellow solid (39 mg, 51%). ¹H NMR δ (400 MHz, d₆-DMSO) 8.62 (d, J 2.5 Hz, 1H), 8.38 (dd, J 8.4, 2.5 Hz, 1H), 8.35 (d, J 7.4 Hz, 1H), 8.20 (s, 1H), 7.96 (s, 1H), 7.74 (d, J 8.4 Hz, 1H), 6.78 (d, J 2.3 Hz, 1H), 6.47 (dd, J 7.4, 2.3 Hz, 1H), 6.06 (br s, 2H), 3.16 (s, 3H), 2.72 (s, 3H). LCMS (APCI⁺) 389 (MH⁺, 100%). Anal. Calcd for C₁₆H₁₆N₆O₄S.0.25H₂O: C, 48.91; H, 4.23; N, 21.39. Found C, 48.92; H, 4.39; N, 21.08.

### Example 13

### N′-((5-Chloropyrazolo[1,5-a]pyridin-3-yl)methylene)-N,2-dimethyl-5-nitrobenzenesulfonohydrazide (E13)

Step 13.1: A solution of NaNO₂ (27 mg, 0.39 mmol) in water (1 mL) was added dropwise to a solution of pyrazolo[1,5-a]pyridin-5-amine (34) (40 mg, 0.30 mmol) and CuCl (74 mg, 0.75 mmol) in concentrated HCl (1 mL) at 0° C. over 2 min. After 30 min, the reaction mixture was heated to 80° C. for 15 min, and then cooled to room temperature, basified to pH 10 with 1M NaOH, filtered through a plug of celite and washed with CH₂Cl₂. The layers of the filtrate were separated and the aqueous layer extracted with CH₂Cl₂. The combined extracts were dried (Na₂SO₄) and the solvent removed in vacuo. Chromatography (eluting with hexanes:EtOAc 3:1) gave 5-chloropyrazolo[1,5-a]pyridine (39: X=Cl) as a white solid (6 mg, 13%). ¹H NMR δ (400 MHz, CDCl₃) 8.38 (d, J 7.4 Hz, 1H), 7.95 (d, J 2.2 Hz, 1H), 7.53 (d, J 1.8 Hz, 1H), 6.71 (dd, J 7.4, 2.2 Hz, 1H), 6.47 (d, J 1.8 Hz, 1H). LCMS (APCI⁺) 153 (MH⁺ with ³⁵Cl, 100%), 155 (MH⁺ with ³⁷Cl, 30%).

Step 13.2: Reaction of 39 (X=Cl) (6 mg, 0.039 mmol) using the conditions of Step 4.3 gave 5-chloropyrazolo[1,5-a]pyridine-3-carbaldehyde (13: X=Cl, Y=H) as a white solid (7 mg, 100%). ¹H NMR δ (400 MHz, CDCl₃) 10.02 (s, 1H), 8.48 (d, J 7.3 Hz, 1H), 8.38 (s, 1H), 8.33 (d, J 2.3 Hz, 1H), 7.04 (dd, J 7.3, 2.3 Hz, 1H). LCMS (APCI⁺) 181 (MH⁺ with ³⁵Cl, 100%), 183 (MH⁺ with ³⁷Cl, 30%).

Step 13.3: Reaction of 13 (X=Cl, Y=H) (7 mg, 0.039 mmol) using the conditions of Example 5 gave N′-((5-chloropyrazolo[1,5-a]pyridin-3-yl)methylene)-N,2-dimethyl-5-nitro-benzenesulfonohydrazide (E13) as a yellow solid (13 mg, 81%). ¹H NMR δ (400 MHz, CDCl₃) 9.01 (d, J 2.4 Hz, 1H), 8.32-8.38 (m, 2H), 8.04 (s, 1H), 7.89 (s, 1H), 7.56 (d, J 1.9 Hz, 1H), 7.53 (d, J 8.4 Hz, 1H), 6.83 (dd, J 7.3, 2.3 Hz, 1H), 3.42 (s, 3H), 2.75 (s, 3H). LCMS (APCI⁺) 408 (MH⁺ with ³⁵Cl, 100%), 410 (MH⁺ with ³⁷Cl, 25%). Anal. Calcd for C₁₆H₁₄ClN₅O₄S: C, 47.12; H, 3.46; N, 17.17. Found C, 47.06; H, 3.53; N, 16.97.

### Example 14

### N′-((5-Bromopyrazolo[1,5-a]pyridin-3-yl)methylene)-N,2-dimethyl-5-nitrobenzenesulfonohydrazide (E14)

Step 14.1: A solution of NaNO₂ (189 mg, 2.74 mmol) in water (3 mL) was added dropwise to a solution of the trifluoroacetate salt of ethyl 5-aminopyrazolo[1,5-a]pyridine-3-carboxylate (33) (374 mg, 1.82 mmol) in concentrated HBr (2 mL) at 0° C. over 2 min. After 10 min, a solution of CuBr (523 mg, 3.65 mmol) in concentrated HBr (2 mL) was added, then the reaction mixture heated to 50° C. for 15 min until gas evolution ceased. Then the reaction mixture was basified to pH 2 with 1M NaOH and extracted twice with CH₂Cl₂. The combined extracts were dried (Na₂SO₄) and the solvent removed in vacuo. Chromatography (eluting with hexanes:EtOAc 9:1) gave ethyl 5-bromopyrazolo[1,5-a]pyridine-3-carboxylate (38: X=Br) as a yellow solid (326 mg, 66%). ¹H NMR δ (400 MHz, CDCl₃) 8.53-8.20 (m, 3H), 7.03 (dd, J 7.3, 2.0 Hz, 1H), 4.39 (q, J 7.1 Hz, 2H), 1.42 (t, J 7.1 Hz, 3H). LCMS (APCI⁺) 269 (MH⁺ with ⁷⁹Br, 100%), 271 (MH⁺ with ⁸¹Br, 90%).

Step 14.2: Reaction of 38 (X=Br) (426 mg, 1.58 mmol) using the conditions of Step 4.2 gave 5-bromopyrazolo[1,5-a]pyridine (12: X=Br, Y=H) as a brown solid (304 mg, 97%). ¹H NMR δ (400 MHz, CDCl₃) 8.34 (d, J 7.4 Hz, 1H), 7.95 (d, J 2.1 Hz, 1H), 7.73 (d, J 1.8 Hz, 1H), 6.84 (dd, J 7.4, 2.1 Hz, 1H), 6.48 (d, J 1.8 Hz, 1H). LCMS (APCI⁺) 197 (MH⁺ with ⁷⁹Br, 100%), 199 (MH⁺ with ⁸¹Br, 90%).

Step 14.3: Reaction of 12 (X=Br, Y=H) (304 mg, 1.54 mmol) using the conditions of Step 4.3 gave 5-bromopyrazolo[1,5-a]pyridine-3-carbaldehyde (13: X=Br, Y=H) as a brown solid (323 mg, 93%). ¹H NMR δ (400 MHz, CDCl₃) 10.02 (s, 1H), 8.51 (d, J 2.1 Hz, 1H), 8.42 (d, J 7.3 Hz, 1H), 8.37 (s, 1H), 7.16 (dd, J 7.3, 2.1 Hz, 1H). LCMS (APCI⁺) 225 (MH⁺ with ⁷⁹Br, 100%), 227 (MH⁺ with ⁸¹Br, 95%).

Step 14.4: Reaction of 13 (X=Br, Y=H) (22 mg, 0.10 mmol) using the conditions of Example 1 gave N′-((5-bromopyrazolo[1,5-a]pyridin-3-yl)methylene)-N,2-dimethyl-5-nitro-benzenesulfonohydrazide (E14) as a yellow solid (43 mg, 98%). ¹H NMR δ (400 MHz, CDCl₃) 9.01 (d, J 2.4 Hz, 1H), 8.36 (dd, J 8.4, 2.4 Hz, 1H), 8.28 (d, J 7.3 Hz, 1H), 8.03 (s, 1H), 7.89 (s, 1H), 7.76 (d, J 2.2 Hz, 1H), 7.53 (d, J 8.4 Hz, 1H), 6.94 (dd, J 7.3, 2.2 Hz, 1H), 3.43 (s, 3H), 2.74 (s, 3H). LCMS (APCI⁺) 452 (MH⁺ with ⁷⁹Br, 80%), 454 (MH⁺ with ⁸¹Br, 100%). Anal. Calcd for C₁₆H₁₄BrN₅O₄S: C, 42.49; H, 3.12; N, 15.48. Found C, 42.77; H, 3.07; N, 15.47.

### Example 15

### N′-((5-Iodopyrazolo[1,5-a]pyridin-3-yl)methylene)-N,2-dimethyl-5-nitro-benzenesulfonohydrazide (E15)

Step 15.1: NaNO₂ (36 mg, 0.52 mmol) was added to a solution of the trifluoroacetate salt of ethyl 5-aminopyrazolo[1,5-a]pyridine-3-carboxylate (33) (150 mg, 0.40 mmol) in concentrated HCl (3 mL), H₂SO₄ (1 mL) and water (3 mL) at 0° C. After 1 h, urea (2.4 mg, 0.04 mmol) was added, then after a further 15 min, a solution of KI (132 mg, 0.80 mmol) in water (3 mL) was added. After 1 h at room temperature, the reaction mixture was basified to pH 3 with 1M NaOH and extracted twice with CH₂Cl₂. The combined extracts were dried (Na₂SO₄) and the solvent removed in vacuo. Chromatography (eluting with hexanes:EtOAc 5:1) gave ethyl 5-iodopyrazolo[1,5-a]pyridine-3-carboxylate (38: X=I) as a pale yellow solid (57 mg, 45%). ¹H NMR δ (400 MHz, CDCl₃) 8.59 (dd, J 1.9, 0.7 Hz, 1H), 8.33 (s, 1H), 8.23 (dd, J 7.2, 0.7 Hz, 1H), 7.18 (dd, J 7.2, 1.9 Hz, 1H), 4.39 (q, J 7.1 Hz, 2H), 1.41 (t, J 7.1 Hz, 3H). LCMS (APCI⁺) 317 (MH⁺, 100%).

Step 15.2: Reaction of 38 (X=I) (57 mg, 0.18 mmol) using the conditions of Step 4.2 gave 5-iodopyrazolo[1,5-a]pyridine (12: X=I, Y=H) as a pale brown solid (42 mg, 95%). ¹H NMR δ (400 MHz, CDCl₃) 8.21 (d, J 7.3 Hz, 1H), 7.95 (d, J 1.7 Hz, 1H), 7.90 (d, J 2.2 Hz, 1H), 6.95 (dd, J 7.3, 1.7 Hz, 1H), 6.44 (d, J 2.2 Hz, 1H). LCMS (APCI⁺) 245 (MH⁺, 100%).

Step 15.3: Reaction of 12 (X=I, Y=H) (42 mg, 0.17 mmol) using the conditions of Step 4.3 gave 5-iodopyrazolo[1,5-a]pyridine-3-carbaldehyde (13: X=I, Y=H) as a pale brown solid (45 mg, 96%). ¹H NMR δ (400 MHz, CDCl₃) 10.02 (s, 1H), 8.74 (d, J 1.7 Hz, 1H), 8.33 (s, 1H), 8.28 (d, J 7.2 Hz, 1H), 7.31 (dd, J 7.2, 1.7 Hz, 1H). LCMS (APCI⁺) 273 (MH⁺, 100%).

Step 15.4: Reaction of 13 (X=I, Y=H) (45 mg, 0.17 mmol) using the conditions of Example 1 gave N′-((5-iodopyrazolo[1,5-a]pyridin-3-yl)methylene)-N,2-dimethyl-5-nitrobenzenesulfonohydrazide (E15) as a yellow solid (60 mg, 72%). ¹H NMR δ (400 MHz, CDCl₃) 8.99 (d, J 2.4 Hz, 1H), 8.36 (dd, J 8.4, 2.4 Hz, 1H), 8.16 (dd, J 7.2, 0.7 Hz, 1H), 8.05 (m, 1H), 8.00 (s, 1H), 7.90 (s, 1H), 7.54 (d, J 8.4 Hz, 1H), 7.09 (dd, J 7.2, 1.9 Hz, 1H), 3.43 (s, 3H), 2.75 (s, 3H). LCMS (APCI⁺) 500 (MH⁺, 100%). Anal. Calcd for C₁₆H₁₄1N₅O₄S.0.15 hexanes: C, 39.63; H, 3.17; N, 13.67. Found C, 39.63; H, 2.95; N, 13.69.

### Example 16

### N,2-Dimethyl-5-nitro-N′-((5-vinylpyrazolo[1,5-a]pyridin-3-yl)methylene)-benzenesulfonohydrazide (E16)

Step 16.1: Pd(PPh₃)₄ (26 mg, 0.022 mmol) was added to a solution of 5-bromopyrazolo[1,5-a]pyridine-3-carbaldehyde (40) (50 mg, 0.22 mmol) and tributyl(vinyl)tin (84 μL, 0.29 mmol) in toluene (10 mL) which had been deoxygenated by bubbling N₂ through it. After refluxing for 2 h, the solvent was removed in vacuo. Chromatography (eluting with hexanes:EtOAc 9:1 to 85:15) gave 5-vinylpyrazolo[1,5-a]-pyridine-3-carbaldehyde (41: X=CHCH₂) as a pale yellow solid (35 mg, 92%). ¹H NMR δ (400 MHz, CDCl₃) 10.03 (s, 1H), 8.49 (d, J 7.2 Hz, 1H), 8.34 (s, 1H), 8.19 (d, J 1.9 Hz, 1H), 7.18 (dd, J 7.2, 1.9 Hz, 1H), 6.80 (dd, J 17.5, 10.9 Hz, 1H), 5.97 (d, J 17.5 Hz, 1H), 5.55 (d, J 10.9 Hz, 1H). LCMS (APCI⁺) 173 (MH⁺, 100%).

Step 16.2: Reaction of 41 (X=CHCH₂) (35 mg, 0.20 mmol) using the conditions of Example 1 gave N,2-dimethyl-5-nitro-N′-((5-vinylpyrazolo[1,5-a]pyridin-3-yl)methylene)benzenesulfonohydrazide (E16) as a yellow solid (36 mg, 44%). ¹H NMR δ (400 MHz, CDCl₃) 8.98 (d, J 2.4 Hz, 1H), 8.37 (d, J 7.3 Hz, 1H), 8.29 (dd, J 8.4, 2.4 Hz, 1H), 8.02 (s, 1H), 7.97 (s, 1H), 7.74 (d, J 1.9 Hz, 1H), 7.49 (d, J 8.4 Hz, 1H), 7.03 (dd, J 7.3, 1.9 Hz, 1H), 6.67 (dd, J 17.5, 10.9 Hz, 1H), 5.82 (d, J 17.5 Hz, 1H), 5.48 (d, J 10.9 Hz, 1H), 3.41 (s, 3H), 2.75 (s, 3H). LCMS (APCI⁺) 400 (MH⁺, 100%). Anal. Calcd for C₁₈H₁₇N₅O₄S: C, 54.13; H, 4.29; N, 17.53. Found C, 54.42; H, 4.29; N, 17.38.

### Example 17

### N′-((5-Cyclopropylpyrazolo[1,5-a]pyridin-3-yl)methylene)-N,2-dimethyl-6-nitrobenzenesulfonohydrazide (E17)

Step 17.1: Reaction of 40 (60 mg, 0.27 mmol) and tributyl(cyclopropyl)stannane (115 mg, 0.35 mmol) using the conditions of Step 16.1 gave 5-cyclopropylpyrazolo[1,5-a]pyridine-3-carbaldehyde (41: X=^{c}Pr) as a pale yellow solid (37 mg, 74%). ¹H NMR δ (400 MHz, CDCl₃) 9.98 (s, 1H), 8.41 (dd, J 7.2, 0.7 Hz, 1H), 8.30 (s, 1H), 7.97 (d, J 1.9 Hz, 1H), 6.74 (dd, J 7.2, 1.9 Hz, 1H), 2.02 (m, 1H), 1.15 (m, 2H), 0.89 (m, 2H). LCMS (APCI⁺) 187 (MH⁺, 100%).

Step 17.2: Reaction of 41 (X=^{c}Pr) (37 mg, 0.20 mmol) using the conditions of Example 1 gave N′-((5-cyclopropylpyrazolo[1,5-a]pyridin-3-yl)methylene)-N,2-dimethyl-5-nitrobenzenesulfonohydrazide (E17) as a yellow solid (37 mg, 45%). ¹H NMR δ (400 MHz, CDCl₃) 8.98 (d, J 2.4 Hz, 1H), 8.32-8.24 (m, 2H), 8.00-7.96 (m, 2H), 7.62 (d, J 1.9 Hz, 1H), 7.49 (d, J 8.4 Hz, 1H), 6.53 (dd, J 7.2, 2.0 Hz, 1H), 3.39 (s, 3H), 2.75 (s, 3H), 1.90 (m, 1H), 1.10 (m, 2H), 0.73 (m, 2H). LCMS (APCI⁺) 414 (MH⁺, 100%). Anal. Calcd for C₁₉H₁₉N₅O₄S: C, 55.20; H, 4.63; N, 16.94. Found C, 54.80; H, 4.57; N, 16.65.

### Example 18

### N′-((5-Ethynylpyrazolo[1,5-a]pyridin-3-yl)methylene)-N,2-dimethyl-5-nitrobenzenesulfonohydrazide (E18)

Step 18.1: Ethynyltrimethylsilane (75 μL, 0.53 mmol) was added to a solution of 40 (60 mg, 0.27 mmol), CuI (5.1 mg, 27 μmol) and (Ph₃P)₂PdCl₂ (9.4 mg, 13 μmol) in DMF (3 mL) and NEt₃ (3 mL) which had been deoxygenated by bubbling N₂ through it. After heating to 60° C. for 2 h the solvents were removed in vacuo, then K₂CO₃ (111 mg, 0.80 mmol) and MeOH (10 mL) were added and the reaction stirred for a further 2 h. The solvent was removed in vacuo, and the residue taken up in water and extracted twice with CH₂Cl₂. The combined extracts were dried (Na₂SO₄) and the solvent removed in vacuo. Chromatography (eluting with hexanes:EtOAc 4:1) gave 5-ethynylpyrazolo[1,5-a]pyridine-3-carbaldehyde (42) as a white solid (14 mg, 31%). ¹H NMR δ (400 MHz, CDCl₃) 10.05 (s, 1H), 8.50 (dd, J 7.1, 0.9 Hz, 1H), 8.43 (m, 1H), 8.40 (s, 1H), 7.07 (dd, J 7.1, 1.8 Hz, 1H), 3.36 (s, 1H). LCMS (APCI⁺) 171 (MH⁺, 100%).

Step 18.2: Reaction of 42 (14 mg, 0.08 mmol) using the conditions of Example 1 gave N′-((5-ethynylpyrazolo[1,5-a]pyridin-3-yl)methylene)-N,2-dimethyl-5-nitrobenzenesulfonohydrazide (E18) as a yellow solid (24 mg, 73%). ¹H NMR δ (400 MHz, CDCl₃) 9.04 (d, J 2.4 Hz, 1H), 8.39-8.29 (m, 2H), 8.05 (s, 1H), 7.90 (s, 1H), 7.64 (m, 1H), 7.52 (d, J 8.4 Hz, 1H), 6.86 (dd, J 7.2, 1.8 Hz, 1H), 3.44 (s, 3H), 3.29 (s, 1H), 2.73 (s, 3H). LCMS (APCI⁺) 398 (MH⁺, 100%). Anal. Calcd for C₁₈H₁₅N₅O₄S: C, 54.40; H, 3.80; N, 17.62. Found C, 54.37; H, 3.87; N, 17.26.

### Example 19

### N′-((5-Cyanopyrazolo[1,5-a]pyridin-3-yl)methylene)-N-methyl-3-nitro-benzenesulfonohydrazide (E19)

Reaction of 3-formylpyrazolo[1,5-a]pyridine-5-carbonitrile (17) (30 mg, 0.18 mmol) and 3-nitrobenzenesulfonyl chloride (78 mg, 0.35 mmol) using the conditions of Example 1 gave N′-((5-cyanopyrazolo[1,5-a]pyridin-3-yl)methylene)-N-methyl-3-nitrobenzenesulfonohydrazide (E19) as a yellow solid (53 mg, 79%). ¹H NMR δ (400 MHz, CDCl₃) 8.71 (t, J 1.9 Hz, 1H), 8.57 (d, J 7.2 Hz, 1H), 8.48 (m, 1H), 8.40 (s, 1H), 8.29 (d, J 7.8 Hz, 1H), 8.21 (s, 1H), 8.02 (s, 1H), 7.83 (t, J 8.0 Hz, 1H), 7.05 (dd, J 7.2, 1.9 Hz, 1H), 3.32 (s, 3H). LCMS (APCI⁺) 385 (MH⁺, 100%). Anal. Calcd for C₁₆H₁₂N₆O₄S.0.33 CH₂Cl₂: C, 47.56; H, 3.09; N, 20.38. Found C, 47.72; H, 3.13; N, 20.40.

### Example 20

### 3-Cyano-N′-((5-cyanopyrazolo[1,5-a]pyridin-3-yl)methylene)-N-methyl-benzenesulfonohydrazide (E20)

Reaction of 17 (30 mg, 0.18 mmol) and 3-cyanobenzenesulfonyl chloride (71 mg, 0.35 mmol) using the conditions of Example 1 gave 3-cyano-N′-((5-cyanopyrazolo[1,5-a]pyridin-3-yl)methylene)-N-methylbenzenesulfonohydrazide (E20) as a yellow solid (49 mg, 77%). ¹H NMR δ (400 MHz, CDCl₃) 8.57 (dd, J 7.2, 1.0 Hz, 1H), 8.33 (dd, J 1.8, 1.0 Hz, 1H), 8.17-8.24 (m, 2H), 8.12 (t, J 1.4 Hz, 1H), 8.01 (s, 1H), 7.91 (dt, J 7.8, 1.4 Hz, 1H), 7.76 (t, J 7.8 Hz, 1H), 7.04 (dd, J 7.2, 1.8 Hz, 1H), 3.31 (s, 3H). LCMS (APCI⁺) 365 (MH⁺, 100%). Anal. Calcd for C₁₇H₁₂N₆O₂S.0.25H₂O: C, 55.35; H, 3.42; N, 22.78. Found C, 55.46; H, 3.41; N, 22.60.

### Example 21

### 5-Cyano-N′-((5-cyanopyrazolo[1,5-a]pyridin-3-yl)methylene)-N,2-dimethylbenzenesulfonohydrazide (E21)

Step 21.1: 3-Amino-4-methylbenzonitrile (52: X=Me, Y=CN) (407 mg, 3.09 mmol) was suspended in concentrated HCl (3 mL) at 0° C. A solution of NaNO₂ (320 mg, 4.64 mmol) in water (1 mL) was added dropwise over 5 mins, and the solution stirred for 45 mins. Meanwhile, AcOH (3 mL) was saturated with SO₂, then CuCl₂.2H₂O (158 mg, 0.93 mmol) was added and SO₂ bubbled through for a further 5 mins. The AcOH mixture was cooled to 5° C., then the diazonium solution added over 5 mins. The resulting mixture was stirred for a further 1.5 h, then the precipitate filtered off, washed with a little water and dried to leave 5-cyano-2-methylbenzenesulfonyl chloride (53: X=Me, Y=CN) as a yellow solid (167 mg, 25%). ¹H NMR δ (400 MHz, CDCl₃) 8.36 (d, J 1.7 Hz, 1H), 7.87 (dd, J 7.9, 1.7 Hz, 1H), 7.58 (d, J 7.9 Hz, 1H), 2.88 (s, 3H). LCMS (APCI⁻) 196 (M-Cl+O, 100%).

Step 21.2: Reaction of 17 (30 mg, 0.18 mmol) and 53 (X=Me, Y=CN) (45 mg, 0.21 mmol) using the conditions of Example 1 gave 5-cyano-N′-((5-cyanopyrazolo[1,5-a]pyridin-3-yl)methylene)-N,2-dimethylbenzenesulfonohydrazide (E21) as a yellow solid (32 mg, 48%). ¹H NMR δ (400 MHz, d₆-DMSO) 8.96 (dd, J 7.2, 1.0 Hz, 1H), 8.41 (s, 1H), 8.32 (d, J 1.8 Hz, 1H), 8.21 (dd, J 1.9, 1.0 Hz, 1H), 8.16 (s, 1H), 8.05 (dd, J 8.0, 1.8 Hz, 1H), 7.68 (d, J 8.0 Hz, 1H), 7.32 (dd, J 7.2, 1.9 Hz, 1H), 3.37 (s, 3H), 2.68 (s, 3H). LCMS (APCI⁺) 379 (MH⁺, 100%). Anal. Calcd for C₁₈H₁₄N₆O₂S.0.2H₂O: C, 56.59; H, 3.80; N, 22.00. Found C, 56.57; H, 3.86; N, 22.02.

### Example 22

### N′-((5-Cyanopyrazolo[1,5-a]pyridin-3-yl)methylene)-N-methyl-3-(trifluoromethyl)benzenesulfonohydrazide (E22)

Reaction of 17 (30 mg, 0.18 mmol) and 3-(trifluoromethyl)benzenesulfonyl chloride (86 mg, 0.35 mmol) using the conditions of Example 1 gave N′-((5-cyanopyrazolo[1,5-a]pyridin-3-yl)methylene)-N-methyl-3-(trifluoromethyl)benzenesulfonohydrazide (E22) as a yellow solid (64 mg, 90%). ¹H NMR δ (400 MHz, CDCl₃) 8.56 (m, 1H), 8.38 (s, 1H), 8.20 (s, 1H), 8.08-8.16 (m, 2H), 8.01 (s, 1H), 7.89 (d, J 7.8 Hz, 1H), 7.76 (t, J 7.8 Hz, 1H), 7.04 (dd, J 7.2, 1.8 Hz, 1H), 3.29 (s, 3H). LCMS (APCI⁺) 408 (MH⁺, 100%). Anal. Calcd for C₁₇H₁₂F₃N₅O₂S: C, 50.12; H, 2.97; N, 17.19. Found C, 50.37; H, 3.00; N, 17.04.

### Example 23

### N′-((5-Cyanopyrazolo[1,5-a]pyridin-3-yl)methylene)-N,2-dimethyl-5-(trifluoromethyl)benzenesulfonohydrazide (E23)

Step 23.1: 4-Methylbenzotrifluoride (54: X=Me, Y=CF₃) (250 mg, 1.56 mmol) was added to ClSO₃H (0.31 mL, 4.7 mmol) at 0° C. over 5 mins. After 10 mins, the reaction mixture was heated to 100° C. for 2 h, and then cooled to room temperature. The solution was poured onto ice, extracted twice with CH₂Cl₂, the combined organic layers were washed with water, dried (Na₂SO₄) and the solvent removed in vacuo to leave 2-methyl-5-(trifluoromethyl)benzenesulfonyl chloride 53 (X=Me, Y=CF₃) as a colourless oil (150 mg, 37%). ¹H NMR δ (400 MHz, CDCl₃) 8.33 (m, 1H), 7.86 (dd, J 8.0, 1.4 Hz, 1H), 7.58 (d, J 8.0 Hz, 1H), 2.87 (s, 3H). LCMS (APCI⁻) 239 (M-Cl+O, 100%).

Step 23.2: Reaction of 17 (30 mg, 0.18 mmol) and 53 (X=Me, Y=CF₃) (54 mg, 0.21 mmol) using the conditions of Example 1 gave N′-((5-cyanopyrazolo[1,5-a]pyridin-3-yl)methylene)-N,2-dimethyl-5-(trifluoromethyl)benzenesulfonohydrazide (E23) as a yellow solid (36 mg, 49%). ¹H NMR δ (400 MHz, CDCl₃) 8.51 (dd, J 7.2, 0.9 Hz, 1H), 8.33 (m, 1H), 8.16 (s, 1H), 8.02 (dd, J 1.8, 0.9 Hz, 1H), 7.87 (s, 1H), 7.79 (dd, J 8.0, 1.6 Hz, 1H), 7.51 (d, J 8.0 Hz, 1H), 6.97 (dd, J 7.2, 1.8 Hz, 1H), 3.41 (s, 3H), 2.73 (s, 3H). LCMS (APCI⁺) 422 (MH⁺, 100%). Anal. Calcd for C₁₈H₁₄F₃N₅O₂S: C, 51.30; H, 3.35; N, 16.62. Found C, 51.15; H, 3.34; N, 16.48.

### Example 24

### N′-((5-Cyanopyrazolo[1,5-a]pyridin-3-yl)methylene)-N,2-dimethylbenzenesulfonohydrazide (E24)

Reaction of 17 (30 mg, 0.18 mmol) and 2-methylbenzenesulfonyl chloride (51 μL, 0.35 mmol) using the conditions of Example 1 gave N′-((5-cyanopyrazolo[1,5-a]pyridin-3-yl)-methylene)-N,2-dimethylbenzenesulfonohydrazide (E24) as a yellow solid (50 mg, 81%). ¹H NMR δ (400 MHz, CDCl₃) 8.46 (dd, J 7.2, 0.9 Hz, 1H), 8.24 (m, 1H), 8.12 (s, 1H), 7.86 (dd, J 1.8, 0.9 Hz, 1H), 7.76 (s, 1H), 7.57-7.62 (m, 2H), 7.34 (m, 1H), 6.92 (dd, J 7.2, 1.8 Hz, 1H), 3.45 (s, 3H), 2.60 (s, 3H). LCMS (APCI⁺) 354 (MH⁺, 100%). Anal. Calcd for C₁₇H₁₅N₅O₂S: C, 57.78; H, 4.28; N, 19.82. Found C, 57.89; H, 4.29; N, 20.02.

### Example 25

### N′-((5-Cyanopyrazolo[1,5-a]pyridin-3-yl)methylene)-N-methyl-4-nitro-benzenesulfonohydrazide (E25)

Reaction of 17 (30 mg, 0.18 mmol) and 4-nitrobenzenesulfonyl chloride (78 mg, 0.35 mmol) using the conditions of Example 1 gave N′-((5-cyanopyrazolo[1,5-a]pyridin-3-yl)methylene)-N-methyl-4-nitrobenzenesulfonohydrazide (E25) as a yellow solid (59 mg, 88%). ¹H NMR δ (400 MHz, d₆-DMSO) 8.98 (dd, J 7.2, 1.0 Hz, 1H), 8.52 (dd, J 1.9, 1.0 Hz, 1H), 8.48 (s, 1H), 8.42 (d, J 9.0 Hz, 2H), 8.21 (s, 1H), 8.16 (d, J 9.0 Hz, 2H), 7.36 (dd, J 7.2, 1.9 Hz, 1H), 3.23 (s, 3H). LCMS (APCI⁺) 385 (MH⁺, 100%). Anal. Calcd for C₁₆H₁₂N₆O₄S: C, 50.00; H, 3.15; N, 21.86. Found C, 50.25; H, 3.19; N, 22.01.

### Example 26

### N′-((5-Cyanopyrazolo[1,5-a]pyridin-3-yl)methylene)-N,2-dimethyl-4-nitrobenzenesulfonohydrazide (E26)

Reaction of 17 (30 mg, 0.18 mmol) and 2-methyl-4-nitrobenzenesulfonyl chloride (92 mg, 0.35 mmol) [A. Courtin, Helv. Chim. Acta 1976, 59 (2), 379] using the conditions of Example 1 gave N′-((5-cyanopyrazolo[1,5-a]pyridin-3-yl)methylene)-N,2-dimethyl-4-nitrobenzenesulfonohydrazide (E26) as a yellow solid (41 mg, 59%). ¹H NMR δ (400 MHz, d₆-DMSO) 8.95 (dd, J 7.2, 1.0 Hz, 1H), 8.40 (s, 1H), 8.32 (s, 1H), 8.27 (m, 2H), 8.17 (s, 1H), 8.13 (dd, J 1.9, 1.0 Hz, 1H), 7.31 (dd, J 7.2, 1.9 Hz, 1H), 3.41 (s, 3H), 2.71 (s, 3H). LCMS (APCI⁺) 399 (MH⁺, 100%). Anal. Calcd for C₁₇H₁₄N₆O₄S: C, 51.24; H, 3.54; N, 21.09. Found C, 50.95; H, 3.55; N, 21.10.

### Example 27

### N′-((5-Cyanopyrazolo[1,5-a]pyridin-3-yl)methylene)-5-fluoro-N,2-dimethylbenzenesulfonohydrazide (E27)

Reaction of 17 (30 mg, 0.18 mmol) and 5-fluoro-2-methylbenzenesulfonyl chloride (73 mg, 0.35 mmol) using the conditions of Example 1 gave N′-((5-cyanopyrazolo[1,5-a]pyridin-3-yl)methylene)-5-fluoro-N,2-dimethylbenzenesulfonohydrazide (E27) as a yellow solid (51 mg, 78%). ¹H NMR δ (400 MHz, CDCl₃) 8.50 (dd, J 7.2, 0.9 Hz, 1H), 8.15 (s, 1H), 7.98 (dd, J 1.8, 0.9 Hz, 1H), 7.85 (dd, J 8.4, 2.7 Hz, 1H), 7.82 (s, 1H), 7.26-7.37 (m, 2H), 6.96 (dd, J 7.2, 1.8 Hz, 1H), 3.42 (s, 3H), 2.59 (s, 3H). LCMS (APCI⁺) 372 (MH⁺, 100%). Anal. Calcd for C₁₇H₁₄FN₅O₂S: C, 54.98; H, 3.80; N, 18.86. Found C, 55.28; H, 3.80; N, 19.18.

### Example 28

### 5-Bromo-N′-((5-cyanopyrazolo[1,5-a]pyridin-3-yl)methylene)-N,2-dimethylbenzenesulfonohydrazide (E28)

Reaction of 17 (30 mg, 0.18 mmol) and 5-bromo-2-methylbenzenesulfonyl chloride (57 mg, 0.21 mmol) [H.-W. Kleemann et al., DE 19832429 (2000)] using the conditions of Example 1 gave 5-bromo-N′-((5-cyanopyrazolo[1,5-a]pyridin-3-yl)methylene)-N,2-dimethylbenzenesulfonohydrazide (E28) as a yellow solid (47 mg, 62%). ¹H NMR δ (400 MHz, CDCl₃) 8.51 (dd, J 7.2, 1.0 Hz, 1H), 8.23 (d, J 2.1 Hz, 1H), 8.16 (s, 1H), 8.03 (dd, J 1.8, 1.0 Hz, 1H), 7.84 (s, 1H), 7.67 (dd, J 8.2, 2.1 Hz, 1H), 7.24 (d, J 8.2 Hz, 1H), 6.98 (dd, J 7.2, 1.8 Hz, 1H), 3.40 (s, 3H), 2.59 (s, 3H). LCMS (APCI⁺) 432 (MH⁺ with ⁷⁹Br, 100%), 434 (MH⁺ with ⁸¹Br, 100%). Anal. Calcd for C₁₇H₁₄BrN₆O₂S.0.05 hexanes: C, 47.59; H, 3.39; N, 16.04. Found C, 47.72; H, 3.42; N, 16.32.

### Example 29

### 3-Bromo-N′-((5-cyanopyrazolo[1,5-a]pyridin-3-yl)methylene)-N-methyl-benzenesulfonohydrazide (E29)

Reaction of 17 (30 mg, 0.18 mmol) and 3-bromobenzenesulfonyl chloride (54 mg, 0.21 mmol) using the conditions of Example 1 gave 3-bromo-N′-((5-cyanopyrazolo[1,5-a]pyridin-3-yl)methylene)-N-methylbenzenesulfonohydrazide (E29) as a yellow solid (63 mg, 86%). ¹H NMR δ (400 MHz, CDCl₃) 8.55 (dd, J 7.2, 0.9 Hz, 1H), 8.40 (dd, J 1.8, 0.9 Hz, 1H), 8.20 (s, 1H), 8.01 (t, J 1.8 Hz, 1H), 7.99 (s, 1H), 7.88 (ddd, J 7.9, 1.8, 1.0 Hz, 1H), 7.76 (ddd, J 7.9, 1.8, 1.0 Hz, 1H), 7.48 (t, J 7.9 Hz, 1H), 7.03 (dd, J 7.2, 1.8 Hz, 1H), 3.28 (s, 3H). LCMS (APCI⁺) 418 (MH⁺ with ⁷⁹Br, 100%), 420 (MH⁺ with ⁸¹Br, 100%). Anal. Calcd for C₁₆H₁₂BrN₅O₂S: C, 45.94; H, 2.89; N, 16.74. Found C, 45.88; H, 3.18; N, 16.52.

### Example 30

### Methyl 3-(24 (5-cyanopyrazolo[1,5-a]pyridin-3-yl)methylene)-1-methyl-hydrazinylsulfonyl)-4-methylbenzoate (E30)

Step 30.1: Reaction of toluic acid (54: X=Me, Y=CO₂H) (500 mg, 3.67 mmol) using the conditions of Step 23.1 gave 3-(chlorosulfonyl)-4-methylbenzoic acid 53 (X=Me, Y=CO₂H) as a pale brown solid (746 mg, 87%). ¹H NMR δ (400 MHz, d₆-DMSO) 8.32 (d, J 1.9 Hz, 1H), 7.76 (dd, J 7.8, 1.9 Hz, 1H), 7.25 (d, J 7.8 Hz; 1H), 5.55 (br s, 1H), 2.58 (s, 3H). LCMS (APCI⁻) 233 (M-H⁺ with ³⁸Cl, 100%), 235 (M-H⁺ with ³⁷Cl, 40%).

Step 30.2: 53 (X=Me, Y=CO₂H) (200 mg, 0.85 mmol) was refluxed in SOCl₂ (1.0 mL) for 1 h. The solvent was removed in vacuo, then MeOH (5 mL) added and the solution stirred for 1 h. The solvent was removed in vacuo to leave methyl 3-(chlorosulfonyl)-4-methyl-benzoate (55: X=Me) as a pale brown solid (127 mg, 60%). ¹H NMR δ (400 MHz, CDCl₃) 8.72 (d, J 1.7 Hz, 1H), 8.25 (dd, J 7.9, 1.7 Hz, 1H), 7.52 (d, J 7.9 Hz, 1H), 3.97 (s, 3H), 2.86 (s, 3H). LCMS (APCI⁻) 229 (M-Cl+O, 100%).

Step 30.3: Reaction of 17 (30 mg, 0.18 mmol) and 55 (X=Me) (87 mg, 0.35 mmol) using the conditions of Example 1 gave methyl 3-(2-((5-cyanopyrazolo[1,5-a]pyridin-3-yl)methylene)-1-methylhydrazinylsulfonyl)-4-methylbenzoate (E30) as a yellow solid (53 mg, 74%). ¹H NMR δ (400 MHz, CDCl₃) 8.78 (d, J 1.5 Hz, 1H), 8.49 (d, J 7.2 Hz, 1H), 8.21 (dd, J 7.9, 1.5 Hz, 1H), 8.15 (s, 1H), 7.98 (dd, J 1.8, 0.8 Hz, 1H), 7.84 (s, 1H), 7.45 (d, J 7.9 Hz, 1H), 6.94 (dd, J 7.2, 1.8 Hz, 1H), 3.95 (s, 3H), 3.44 (s, 3H), 2.69 (s, 3H). LCMS (APCI⁺) 412 (MH⁺, 100%). Anal. Calcd for C₁₆H₁₇N₆O₄S: C, 55.47; H, 4.16; N, 17.02. Found C, 55.72; H, 4.26; N, 17.18.

### Example 31

### N′-((5-Cyanopyrazolo[1,5-a]pyridin-3-yl)methylene)-N,2-dimethyl-5-(methylsulfonyl)benzenesulfonohydrazide (E31)

Step 31.1: Reaction of 1-methyl-4-(methylsulfonyl)benzene (54: X=Me, Y=SO₂Me) (250 mg, 1.47 mmol) using the conditions of Step 23.1 gave 2-methyl-5-(methylsulfonyl)benzenesulfonyl chloride 53 (X=Me, Y=SO₂Me) as a white solid (327 mg, 83%). ¹H NMR δ (400 MHz, CDCl₃) 8.62 (d, J 1.9 Hz, 1H), 8.17 (dd, J 8.0, 1.9 Hz, 1H), 7.66 (d, J 8.0 Hz, 1H), 3.12 (s, 3H), 2.91 (s, 3H). LCMS (APCI⁻) 249 (M-Cl+O, 100%).

Step 31.2: Reaction of 17 (30 mg, 0.18 mmol) and 53 (X=Me, Y=SO₂Me) (94 mg, 0.35 mmol) using the conditions of Example 1 gave N′-((5-cyanopyrazolo[1,5-a]pyridin-3-yl)-methylene)-N,2-dimethyl-5-(methylsulfonyl)benzenesulfonohydrazide (E31) as a yellow solid (66 mg, 87%). ¹H NMR δ (400 MHz, CDCl₃) 8.54 (d, J 2.0 Hz, 1H), 8.52 (dd, J 7.2, 1.0 Hz, 1H), 8.21 (dd, J 1.8, 1.0 Hz, 1H), 8.18 (s, 1H), 8.05 (dd, J 8.0, 2.0 Hz, 1H), 7.89 (s, 1H), 7.58 (d, J 8.0 Hz, 1H), 6.99 (dd, J 7.2, 1.8 Hz, 1H), 3.41 (s, 3H), 3.09 (s, 3H), 2.80 (s, 3H). LCMS (APCI⁺) 432 (MH⁺, 100%). Anal. Calcd for C₁₈H₁₇N₅O₄S₂: C, 50.10; H, 3.97; N, 16.23. Found C, 50.35; H, 3.97; N, 16.26.

### Example 32

### N′-((5-Cyanopyrazolo[1,5-a]pyridin-3-yl)methylene)-2-ethyl-N-methyl-5-nitrobenzenesulfonohydrazide (E32)

Reaction of 17 (30 mg, 0.18 mmol) and 2-ethyl-5-nitrobenzenesulfonyl chloride (88 mg, 0.35 mmol) [C. Hansch et al., J. Org. Chem. 1956, 21 (3), 265] using the conditions of Example 1 gave (E32) as a yellow solid (66 mg, 92%). ¹H NMR δ (400 MHz, CDCl₃) 8.89 (d, J 2.4 Hz, 1H), 8.52 (dd, J 7.2, 1.0 Hz, 1H), 8.41 (dd, J 8.5, 2.4 Hz, 1H), 8.18 (s, 1H), 8.07 (dd, J 1.8, 1.0 Hz, 1H), 7.89 (s, 1H), 7.62 (d, J 8.5 Hz, 1H), 6.98 (dd, J 7.2, 1.8 Hz, 1H), 3.44 (s, 3H), 3.21 (q, J 7.5 Hz, 2H), 1.33 (t, J 7.5 Hz, 3H). LCMS (APCI⁺) 413 (MH⁺, 100%). Anal. Calcd for C₁₈H₁₆N₆O₄S: C, 52.42; H, 3.91; N, 20.38. Found C, 52.41; H, 3.93; N, 20.21.

### Example 33

### N′-((5-Cyanopyrazolo[1,5-a]pyridin-3-yl)methylene)-2-isopropyl-N-methyl-5-nitrobenzenesulfonohydrazide (E33)

Step 33.1: Reaction of 1-isopropyl-4-nitrobenzene (54: X=ⁱPr, Y=NO₂) (500 mg, 3.03 mmol) using the conditions of Step 23.1 gave 2-isopropyl-5-nitrobenzenesulfonyl chloride 53 (X=ⁱPr, Y=NO₂) as a pale yellow solid (127 mg, 16%). ¹H NMR δ (400 MHz, CDCl₃) 8.92 (d, J 2.4 Hz, 1H), 8.50 (dd, J 8.7, 2.4 Hz, 1H), 7.81 (d, J 8.7 Hz, 1H), 4.15 (septet, J 6.8 Hz, 1H), 1.40 (d, J 6.8 Hz, 6H). LCMS (APCI⁻) 244 (M-Cl+O, 100%).

Step 33.2: Reaction of 17 (30 mg, 0.18 mmol) and 53 (X=ⁱPr, Y=NO₂) (55 mg, 0.21 mmol) using the conditions of Example 1 gave N′-((5-cyanopyrazolo[1,5-a]pyridin-3-yl)methylene)-2-isopropyl-N-methyl-5-nitrobenzenesulfonohydrazide (E33) as a yellow solid (41 mg, 55%). ¹H NMR δ (400 MHz, CDCl₃) 8.91 (d, J 2.4 Hz, 1H), 8.51 (dd, J 7.2, 1.0 Hz, 1H), 8.44 (dd, J 8.7, 2.4 Hz, 1H), 8.17 (s, 1H), 8.05 (dd, J 1.8, 1.0 Hz, 1H), 7.87 (s, 1H), 7.72 (d, J 8.7 Hz, 1H), 6.97 (dd, J 7.2, 1.8 Hz, 1H), 4.09 (septet, J 6.8 Hz, 1H), 3.44 (s, 3H), 1.25 (d, J 6.8 Hz, 6H). LCMS (APCI⁺) 427 (MH⁺, 100%). Anal. Calcd for C₁₉H₁₈N₆O₄S: C, 53.51; H, 4.25; N, 19.71. Found C, 53.56; H, 4.42; N, 19.84.

### Example 34

### 2-Chloro-N′-((5-cyanopyrazolo[1,5-a]pyridin-3-yl)methylene)-N-methyl-5-nitrobenzenesulfonohydrazide (E34)

Reaction of 17 (30 mg, 0.18 mmol) and 2-chloro-5-nitrobenzenesulfonyl chloride (90 mg, 0.35 mmol) using the conditions of Example 1 gave 2-chloro-N′-((5-cyanopyrazolo[1,5-a]-pyridin-3-yl)methylene)-N-methyl-5-nitrobenzenesulfonohydrazide (E34) as a yellow solid (20 mg, 27%). ¹H NMR δ (400 MHz, CDCl₃) 9.16 (d, J 2.7 Hz, 1H), 8.50 (dd, J 7.2, 0.9 Hz, 1H), 8.40 (dd, J 8.7, 2.7 Hz, 1H), 8.16 (s, 1H), 8.00 (dd, J 1.8, 0.9 Hz, 1H), 7.86 (s, 1H), 7.72 (d, J 8.7 Hz, 1H), 6.96 (dd, J 7.2, 1.8 Hz, 1H), 3.57 (s, 3H). LCMS (APCI⁺) 419 (MH⁺, 100%). Anal. Calcd for C₁₆H₁₁ClN₆O₄S.0.33H₂O: C, 45.24; H, 2.77; N, 19.79. Found C, 45.33; H, 2.79; N, 20.08.

### Example 35

### N′-((5-Cyanopyrazolo[1,5-a]pyridin-3-yl)methylene)-2-methoxy-N-methyl-5-nitrobenzenesulfonohydrazide (E35)

Reaction of 17 (30 mg, 0.18 mmol) and 2-methoxy-5-nitrobenzenesulfonyl chloride (88 mg, 0.35 mmol) using the conditions of Example 1 gave N′-((5-cyanopyrazolo[1,5-a]pyridin-3-yl)methylene)-2-methoxy-N-methyl-5-nitrobenzenesulfonohydrazide (E35) as a yellow solid (56 mg, 77%). ¹H NMR δ (400 MHz, d₆-DMSO) 8.93 (dd, J 7.2, 0.9 Hz, 1H), 8.72 (d, J 2.9 Hz, 1H), 8.51 (dd, J 9.2, 2.9 Hz, 1H), 8.37 (s, 1H), 8.12 (s, 1H), 8.07 (dd, J 1.9, 0.9 Hz, 1H), 7.46 (d, J 9.2 Hz, 1H), 7.29 (dd, J 7.2, 1.9 Hz, 1H), 4.02 (s, 3H), 3.45 (s, 3H). LCMS (APCI⁺) 415 (MH⁺, 100%). Anal. Calcd for C₁₇H₁₄N₆O₅S: C, 49.27; H, 3.41; N, 20.28. Found C, 49.31; H, 3.41; N, 20.09.

### Example 36

### N′-((5-Cyanopyrazolo[1,5-a]pyridin-3-yl)methylene)-2-(dimethylamino)-N-methyl-5-nitrobenzenesulfonohydrazide (E36)

Reaction of 17 (30 mg, 0.18 mmol) and 2-(dimethylamino)-5-nitrobenzenesulfonyl chloride (93 mg, 0.35 mmol) [R. A. Abramovitch et al., J. Org. Chem. 1977, 42 (17), 2920] using the conditions of Example 1 gave N′-((5-cyanopyrazolo[1,5-a]pyridin-3-yl)methylene)-2-(dimethylamino)-N-methyl-5-nitrobenzenesulfonohydrazide (E36) as a yellow solid (22 mg, 29%). ¹H NMR δ (400 MHz, d₆-DMSO) 8.95 (dd, J 7.2, 1.0 Hz; 1H), 8.66 (d, J 2.8 Hz, 1H), 8.39 (s, 1H), 8.28 (dd, J 9.3, 2.8 Hz, 1H), 8.15 (s, 1H), 8.05 (dd, J 1.9, 1.0 Hz, 1H), 7.38 (d, J 9.3 Hz, 1H), 7.30 (dd, J 7.2, 1.9 Hz, 1H), 3.39 (s, 3H), 2.99 (s, 6H). LCMS (APCI⁺) 428 (MH⁺, 100%). Anal. Calcd for C₁₈H₁₇N₇O₄S.0.05 hexanes: C, 50.91; H, 4.13; N, 22.71. Found C, 51.06; H, 4.03; N, 22.91.

### Example 37

### N′-((5-Bromopyrazolo[1,5-a]pyridin-3-yl)methylene)-5-cyano-N,2-dimethylbenzenesulfonohydrazide (E37)

Reaction of 5-bromopyrazolo[1,5-a]pyridine-3-carbaldehyde (40) (30 mg, 0.13 mmol) and 5-cyano-2-methylbenzenesulfonyl chloride (57 mg, 0.26 mmol) using the conditions of Example 1 gave N′-((5-bromopyrazolo[1,5-a]pyridin-3-yl)methylene)-5-cyano-N,2-dimethylbenzenesulfonohydrazide (E37) as a yellow solid (47 mg, 81%). ¹H NMR δ (400 MHz, CDCl₃) 8.43 (d, J 1.7 Hz, 1H), 8.30 (dd, J 7.3, 0.7 Hz, 1H), 8.03 (s, 1H), 7.86 (s, 1H), 7.82 (dd, J 2.2, 0.7 Hz, 1H), 7.77 (dd, J 7.9, 1.7 Hz, 1H), 7.46 (d, J 7.9 Hz, 1H), 6.97 (dd, J 7.3, 2.2 Hz, 1H), 3.41 (s, 3H), 2.70 (s, 3H). LCMS (APCI⁺) 432 (MH⁺ with ⁷⁹Br, 90%), 434 (MH⁺ with ⁸¹Br, 100%). Anal. Calcd for C₁₇H₁₄BrN₅O₂S.H₂O: C, 45.34; H, 3.58; N, 15.22. Found C, 45.26; H, 3.56; N, 14.93.

### Example 38

### N′-((5-Cyanopyrazolo[1,5-a]pyridin-3-yl)methylene)-N-(2-hydroxyethyl)-2-methyl-5-nitrobenzenesulfonohydrazide (E38)

2-Hydroxyethylhydrazine (27 mg, 0.35 mmol) was added to a solution of 3-formylpyrazolo-[1,5-a]pyridine-5-carbonitrile (17) (30 mg, 0.18 mmol) in MeOH (10 mL). After 2 h, NaHCO₃ (59 mg, 0.70 mmol) and 2-methyl-5-nitrobenzenesulfonyl chloride (83 mg, 0.35 mmol) were added and the reaction mixture stirred for a further 3 h. The solvent was removed in vacuo and the residue taken up in CH₂Cl₂ and water. The layers were separated and the aqueous phase extracted with CH₂Cl₂, then the combined organic layers were dried (Na₂SO₄) and the solvent removed in vacuo. Chromatography (eluting with hexanes:EtOAc 2:1 to 1:1 to EtOAc) gave N′-((5-cyanopyrazolo[1,5-a]pyridin-3-yl)methylene)-N-(2-hydroxyethyl)-2-methyl-5-nitrobenzenesulfonohydrazide (E38) as a yellow solid (41 mg, 55%). ¹H NMR δ (400 MHz; d₆-DMSO) 8.96 (dd, J 7.2, 1.0 Hz, 1H), 8.73 (d, J 2.5 Hz, 1H), 8.45-8.38 (m, 3H), 8.08 (dd, J 1.9, 1.0 Hz, 1H), 7.77 (d, J 8.4 Hz, 1H), 7.32 (dd, J 7.2, 1.9 Hz, 1H), 5.05 (t, J 5.8 Hz, 1H), 3.99 (t, J 5.8 Hz, 2H), 3.68 (q, J 5.8 Hz, 2H), 2.69 (s, 3H). LCMS (APCI⁺) 429 (MH⁺, 100%). Anal. Calcd for C₁₈H₁₆N₆O₅S: C, 50.46; H, 3.76; N, 19.62. Found C, 50.09; H, 3.86; N, 19.27.

### Example 39

### N′-((5-Bromopyrazolo[1,5-a]pyridin-3-yl)methylene)-N-(2-hydroxyethyl)-2-methyl-5-nitrobenzenesulfonohydrazide (E39)

Reaction of 5-bromopyrazolo[1,5-a]pyridine-3-carbaldehyde (40) (30 mg, 0.13 mmol) and 2-methyl-5-nitrobenzenesulfonyl chloride (47 mg, 0.20 mmol) using the conditions of Example 38 gave N′-((5-bromopyrazolo[1,5-a]pyridin-3-yl)methylene)-N-(2-hydroxyethyl)-2-methyl-5-nitrobenzenesulfonohydrazide (E39) as a yellow solid (29 mg, 45%). ¹H NMR δ (400 MHz, CDCl₃) 8.91 (d, J 2.4 Hz, 1H), 8.46 (s, 1H), 8.37-8.32 (m, 2H), 8.11 (s, 1H), 7.89 (dd, J 2.1, 0.7 Hz, 1H), 7.54 (d, J 8.4 Hz, 1H), 7.01 (dd, J 7.2, 2.1 Hz, 1H), 3.90 (m, 4H), 2.74 (s, 3H). LCMS (APCI⁺) 482 (MH⁺ with ⁷⁹Br, 100%), 484 (MH⁺ with ⁸¹Br, 95%). Anal. Calcd for C₁₇H₁₆BrN₅O₅S.0.5 EtOAc: C, 43.36; H, 3.83; N, 13.30. Found C, 43.35; H, 3.79; N, 13.08.

### Example 40

### 5-Cyano-N′-((5-cyanopyrazolo[1,5-a]pyridin-3-yl)methylene)-N-(2-hydroxyethyl)-2-methylbenzenesulfonohydrazide (E40)

Reaction of 3-formylpyrazolo[1,5-a]pyridine-5-carbonitrile (17) (30 mg, 0.18 mmol) and 5-cyano-2-methylbenzenesulfonyl chloride (57 mg, 0.26 mmol) using the conditions of Example 38 gave 5-cyano-N′-((5-cyanopyrazolo[1,5-a]pyridin-3-yl)methylene)-N-(2-hydroxyethyl)-2-methylbenzenesulfonohydrazide (E40) as a yellow solid (18 mg, 25%). ¹H NMR δ (400 MHz, CDCl₃) 8.56 (dd, J 7.2, 0.9 Hz, 1H), 8.42 (s, 1H), 8.27 (d, J 1.7 Hz, 1H), 8.23 (s, 1H), 8.00 (dd, J 1.8, 0.9 Hz, 1H), 7.82 (dd, J 7.9, 1.7 Hz, 1H), 7.52 (d, J 7.9 Hz, 1H), 7.02 (dd, J 7.2, 1.8 Hz, 1H), 3.94 (m, 4H), 2.74 (s, 3H), 1.90 (m, 1H). LCMS (APCI⁺) 409 (MH⁺, 100%). Anal. Calcd for C₁₉H₁₆N₆O₃S.0.33 EtOAc: C, 55.78; H, 4.29; N, 19.21. Found C, 55.45; H, 4.05; N, 19.21.

### Example 41

### N-(2-Hydroxyethyl)-2-methyl-5-nitro-N′-(pyrazolo[1,5-a]pyridin-3-ylmethylene)benzenesulfonohydrazide (E41)

Reaction of pyrazolo[1,5-a]pyridine-3-carbaldehyde (13: X=Y=H) (30 mg, 0.21 mmol) and 2-methyl-5-nitrobenzenesulfonyl chloride (53 mg, 0.22 mmol) using the conditions of Example 38 gave N-(2-hydroxyethyl)-2-methyl-5-nitro-N′-(pyrazolo[1,5-a]pyridin-3-ylmethylene)benzenesulfonohydrazide (E41) as a yellow solid (41 mg, 49%). ¹H NMR δ (400 MHz, CDCl₃) 8.93 (d, J 2.4 Hz, 1H), 8.55 (s, 1H), 8.52 (d, J 6.9 Hz, 1H), 8.30 (dd, J 8.4, 2.4 Hz, 1H), 8.14 (s, 1H), 7.93 (d, J 8.8 Hz, 1H), 7.51 (d, J 8.4 Hz, 1H), 7.34 (ddd, J 8.8, 6.9, 1.0 Hz, 1H), 6.96 (td, J 6.9, 1.3 Hz, 1H), 3.93-3.80 (m, 4H), 2.74 (s, 3H), 1.98 (br s, 1H). LCMS (APCI⁺) 404 (MH⁺, 100%). Anal. Calcd for C₁₇H₁₇N₅O₅S: C, 50.61; H, 4.25; N, 17.36. Found C, 50.72; H, 4.52; N, 17.26.

### Example 42

### N′-((5-Cyanopyrazolo[1,5-a]pyridin-3-yl)methylene)-2-methyl-5-nitro-benzenesulfonohydrazide (E42)

3-Formylpyrazolo[1,5-a]pyridine-5-carbonitrile (17) (150 mg, 0.88 mmol) and 2-methyl-5-nitrobenzenesulfonohydrazide (45) (213 mg, 0.92 mmol) were stirred in MeOH (30 mL) for 18 h. The precipitate was filtered off and dried to leave N′-((5-cyanopyrazolo[1,5-a]pyridin-3-yl)methylene)-2-methyl-5-nitrobenzenesulfonohydrazide (E42) as a yellow solid (305 mg, 91%). ¹H NMR δ (400 MHz, d₆-DMSO) 11.99 (s, 1H), 8.94 (dd, J 7.2, 0.9 Hz, 1H), 8.72 (d, J 2.5 Hz, 1H), 8.42 (s, 1H), 8.38 (dd, J 8.4, 2.5 Hz, 1H), 8.20 (s, 1H), 8.10 (dd, J 1.9, 0.9 Hz, 1H), 7.73 (d, J 8.4 Hz, 1H), 7.31 (dd, J 7.2, 1.9 Hz, 1H), 2.77 (s, 3H). LCMS (APCI⁺) 385 (MH⁺, 100%). Anal. Calcd for C₁₆H₁₂N₆O₄S: C, 50.00; H, 3.15; N, 21.86. Found C, 50.21; H, 3.15; N, 21.80.

### Example 43

### N-Benzyl-N′-((5-cyanopyrazolo[1,5-a]pyridin-3-yl)methylene)-2-methyl-5-nitrobenzenesulfonohydrazide (E43)

NaH (6.9 mg, 60% in oil, 0.17 mmol) was added to a solution of E42 (60 mg, 0.16 mmol) in dry DMF (5 mL) at room temperature. After 1 h, benzyl bromide (27 mg, 0.16 mmol) in DMF (0.5 mL) was added. After a further 1 h, the reaction mixture was diluted with water and extracted twice with EtOAc. The combined extracts were washed twice with water then with brine, dried (Na₂SO₄), and the solvent removed in vacuo. Chromatography (eluting with hexanes:EtOAc 4:1 to 2:1) gave N-benzyl-N′-((5-cyanopyrazolo[1,5-a]pyridin-3-yl)methylene)-2-methyl-5-nitrobenzenesulfonohydrazide (E43) as a yellow solid (36 mg, 49%). ¹H NMR δ (400 MHz, CDCl₃) 8.92 (d, J 2.4 Hz, 1H), 8.49 (dd, J 7.2, 0.9 Hz, 1H), 8.38 (dd, J 8.4, 2.4 Hz, 1H), 8.07 (dd, J 1.8, 0.9 Hz, 1H), 8.04 (s, 1H), 7.89 (s, 1H), 7.59 (d, J 8.4 Hz, 1H), 7.38 (m, 4H), 7.31 (m, 1H), 6.97 (dd, J 7.2, 1.8 Hz, 1H), 5.09 (s, 2H), 2.84 (s, 3H). LCMS (APCI⁺) 475 (MH⁺, 100%). Anal. Calcd for C₂₃H₁₈N₆O₄S: C, 58.22; H, 3.82; N, 17.71. Found C, 58.15; H, 3.97; N, 17.45.

### Example 44

### N′-((5-Cyanopyrazolo[1,5-a]pyridin-3-yl)methylene)-N-ethyl-2-methyl-5-nitrobenzenesulfonohydrazide (E44)

Reaction of E42 (60 mg, 0.16 mmol) and iodoethane (25 mg, 0.16 mmol) using the conditions of Example 43 gave N′-((5-cyanopyrazolo[1,5-a]pyridin-3-yl)methylene)-N-ethyl-2-methyl-5-nitrobenzenesulfonohydrazide (E44) as a yellow solid (23 mg, 36%). ¹H NMR δ (400 MHz, CDCl₃) 8.85 (d, J 2.4 Hz, 1H), 8.55 (dd, J 7.2, 0.9 Hz, 1H), 8.36 (dd, J 8.4, 2.4 Hz, 1H), 8.23 (s, 1H), 8.21 (s, 1H), 8.13 (dd, J 1.8, 0.9 Hz, 1H), 7.56 (d, J 8.4 Hz, 1H), 7.01 (dd, J 7.2, 1.8 Hz, 1H), 3.89 (q, J 7.1 Hz, 2H), 2.76 (s, 3H), 1.34 (t, J 7.1 Hz, 3H). LCMS (APCI⁺) 413 (MH⁺, 100%). Anal. Calcd for C₁₈H₁₆N₆O₄S: C, 52.42; H, 3.91; N, 20.38. Found C, 52.66; H, 4.08; N, 20.10.

### Example 45

### N′-((5-Cyanopyrazolo[1,5-a]pyridin-3-yl)methylene)-N-(2-(diethylamino)-ethyl)-2-methyl-5-nitrobenzenesulfonohydrazide (E45)

A suspension of E42 (20 mg, 0.052 mmol), 2-bromo-N,N-diethylethylamine hydrobromide (27 mg, 0.10 mmol) and Cs₂CO₃ (85 mg, 0.26 mmol) in DMF (3 mL) was stirred at room temperature for 2 h. The solution was diluted with water, extracted twice with CH₂Cl₂, the combined extracts were dried (Na₂SO₄) and the solvents removed in vacuo. Chromatography (eluting with CH₂Cl₂: MeOH 99:1 to 98:2) gave N′-((5-cyanopyrazolo[1,5-a]pyridin-3-yl)methylene)-N-(2-(diethylamino)ethyl)-2-methyl-5-nitrobenzenesulfonohydrazide (E45) as a yellow solid (10 mg, 40%). ¹H NMR δ (400 MHz, CDCl₃) 8.88 (d, J 2.4 Hz, 1H), 8.53 (dd, J 7.2, 1.0 Hz, 1H), 8.38 (s, 1H), 8.35 (dd, J 8.4, 2.4 Hz, 1H), 8.21 (s, 1H), 8.10 (s, 1H), 7.56 (d, J 8.4 Hz, 1H), 6.99 (dd, J 7.2, 1.8 Hz, 1H), 3.93 (m, 2H), 2.78 (m, 5H), 2.64 (m, 4H), 1.07 (m, 6H). LCMS (APCI⁺) 484 (MH⁺, 100%). Anal. Calcd for C₂₂H₂₅N₇O₄S.0.5 MeOH: C, 53.99; H, 5.49; N, 19.50. Found C, 54.27; H, 5.51; N, 19.26.

### Example 46

### N′-((5-Cyanopyrazolo[1,5-a]pyridin-3-yl)methylene)-N-(2-(dimethylamino)ethyl)-2-methyl-5-nitrobenzenesulfonohydrazide (E46)

Reaction of E42 (40 mg, 0.10 mmol) and 2-bromo-N,N-dimethylethylamine hydrobromide (36 mg, 0.15 mmol) using the conditions of Example 45 gave N′-((5-cyanopyrazolo[1,5-a]-pyridin-3-yl)methylene)-N-(2-(dimethylamino)ethyl)-2-methyl-5-nitrobenzenesulfonohydrazide (E46) as a yellow solid (22 mg, 47%). ¹H NMR δ (400 MHz, CDCl₃) 8.88 (d, J 2.4 Hz, 1H), 8.54 (dd, J 7.2, 1.0 Hz, 1H), 8.35 (dd, J 8.4, 2.4 Hz, 1H), 8.33 (s, 1H), 8.23 (s, 1H), 8.12 (dd, J 1.9, 1.0 Hz, 1H), 7.56 (d, J 8.4 Hz, 1H), 7.00 (dd, J 7.2, 1.9 Hz, 1H), 3.92 (t, J 6.8 Hz, 2H), 2.78 (s, 3H), 2.61 (t, J 6.8 Hz, 2H), 2.33 (s, 6H). LCMS (APCI⁺) 456 (MH⁺, 100%). Anal. Calcd for C₂₀H₂₁N₇O₄S.0.67 MeOH: C, 52.06; H, 5.00; N, 20.57. Found C, 51.99; H, 4.76; N, 20.45.

### Example 47

### N′-((5-Cyanopyrazolo[1,5-a]pyridin-3-yl)methylene)-2-methyl-N-(2-morpholinoethyl)-5-nitrobenzenesulfonohydrazide (E47)

Reaction of E42 (40 mg, 0.10 mmol) and 4-(2-bromoethyl)morpholine hydrobromide (43 mg, 0.16 mmol) using the conditions of Example 45 gave N′-((5-cyanopyrazolo[1,5-a]pyridin-3-yl)methylene)-2-methyl-N-(2-morpholinoethyl)-5-nitrobenzenesulfonohydrazide (E47) as a yellow solid (41 mg, 79%). ¹H NMR δ (400 MHz, CDCl₃) 8.85 (d, J 2.4 Hz, 1H), 8.55 (dd, J 7.2, 1.0 Hz, 1H), 8.39-8.33 (m, 2H), 8.23 (s, 1H), 8.10 (dd, J 1.8, 1.0 Hz, 1H), 7.57 (d, J 8.5 Hz, 1H), 7.01 (dd, J 7.2, 1.8 Hz, 1H), 3.93 (t, J 6.6 Hz, 2H), 3.70 (m, 4H), 2.78 (s, 3H), 2.67 (t, J 6.6 Hz, 2H), 2.55 (m, 4H). LCMS (APCI⁺) 498 (MH⁺, 100%). Anal. Calcd for C₂₂H₂₃N₇O₅S.0.25H₂O: C, 52.63; H, 4.72; N, 19.53. Found C, 52.61; H, 4.71; N, 19.24.

### Example 48

### N′-((5-Cyanopyrazolo[1,5-a]pyridin-3-yl)methylene)-2-methyl-5-nitro-N-(2-(piperidin-1-yl)ethyl)benzenesulfonohydrazide (E48)

Reaction of E42 (40 mg, 0.10 mmol) and 1-(2-bromoethyl)piperidine hydrobromide (43 mg, 0.16 mmol) using the conditions of Example 45 gave N′-((5-cyanopyrazolo[1,5-a]pyridin-3-yl)methylene)-2-methyl-5-nitro-N-(2-(piperidin-1-yl)ethyl)benzenesulfonohydrazide (E48) as a yellow solid (43 mg, 83%). ¹H NMR δ (400 MHz, CDCl₃) 8.86 (d, J 2.4 Hz, 1H), 8.53 (dd, J 7.2, 0.9 Hz, 1H), 8.39-8.33 (m, 2H), 8.21 (s, 1H), 8.08 (m, 1H), 7.56 (d, J 8.4 Hz, 1H), 6.99 (dd, J 7.2, 1.9 Hz, 1H), 3.96 (t, J 6.5 Hz, 2H), 2.78 (s, 3H), 2.64 (t, J 6.5 Hz, 2H), 2.48 (m, 4H), 1.59 (m, 4H), 1.46 (m, 2H). LCMS (APCI⁺) 496 (MH⁺, 100%). Anal. Calcd for C₂₃H₂₅N₇O₄S: C, 55.74; H, 5.08; N, 19.79. Found C, 56.13; H, 5.39; N, 19.56.

### Example 49

### N′-((5-Cyanopyrazolo[1,5-a]pyridin-3-yl)methylene)-2-methyl-5-nitro-N-(2-(pyrrolidin-1-yl)ethyl)benzenesulfonohydrazide (E49)

Reaction of E42 (40 mg, 0.10 mmol) and 1-(2-bromoethyl)pyrrolidine hydrobromide (54 mg, 0.21 mmol) using the conditions of Example 45 gave N′-((5-cyanopyrazolo[1,5-a]pyridin-3-yl)methylene)-2-methyl-5-nitro-N-(2-(pyrrolidin-1-yl)ethyl)benzenesulfonohydrazide (E49) as a yellow solid (16 mg, 32%). ¹H NMR δ (400 MHz, CDCl₃) 8.88 (d, J 2.4 Hz, 1H), 8.54 (dd, J 7.2, 0.9 Hz, 1H), 8.37-8.32 (m, 2H), 8.22 (s, 1H), 8.11 (dd, J 1.8, 0.9 Hz, 1H), 7.56 (d, J 8.4 Hz, 1H), 6.99 (dd, J 7.2, 1.8 Hz, 1H), 3.98 (m, 2H), 2.83 (m, 2H), 2.78 (s, 3H), 2.64 (m, 4H), 1.82 (m, 4H). LCMS (APCI⁺) 482 (MH⁺, 100%). Anal. Calcd for C₂₂H₂₃N₇O₄S.0.3 EtOAc: C, 54.86; H, 5.06; N, 19.20. Found C, 54.84; H, 5.04; N, 19.25.

### Example 50

### N′-((5-Bromopyrazolo[1,5-a]pyridin-3-yl)methylene)-2-methyl-5-nitro-benzenesulfonohydrazide (E50)

Reaction of 5-bromopyrazolo[1,5-a]pyridine-3-carbaldehyde (40) (200 mg, 0.89 mmol) using the conditions of Example 42 gave N′-((5-bromopyrazolo[1,5-a]pyridin-3-yl)methylene)-2-methyl-5-nitrobenzenesulfonohydrazide (E50) as a yellow solid (333 mg, 86%). ¹H NMR δ (400 MHz, d₆-DMSO) 11.81 (s, 1H), 8.74 (d, J 2.5 Hz, 1H), 8.70 (d, J 7.3 Hz, 1H), 8.40 (dd, J 8.4, 2.5 Hz, 1H), 8.27 (s, 1H), 8.14 (s, 1H), 7.76 (dd, J 2.2, 0.7 Hz, 1H), 7.74 (d, J 8.4 Hz, 1H), 7.15 (dd, J 7.3, 2.2 Hz, 1H), 2.76 (s, 3H). LCMS (APCI⁺) 438 (MH⁺ with ⁷⁹Br, 90%), 440 (MH⁺ with ⁸¹Br, 100%). Anal. Calcd for C₁₅H₁₂BrN₅O₄S: C, 41.11; H, 2.76; N, 15.98. Found C, 41.41; H, 2.90; N, 15.72.

### Example 51

### N′-((5-Bromopyrazolo[1,5-a]pyridin-3-yl)methylene)-2-methyl-5-nitro-N-(2-(piperidin-1-yl)ethyl)benzenesulfonohydrazide hydrochloride (E51)

Reaction of E50 (40 mg, 0.091 mmol) and 1-(2-bromoethyl)piperidine hydrobromide (37 mg, 0.14 mmol) using the conditions of Example 45 gave N′-((5-bromopyrazolo[1,5-a]pyridin-3-yl)methylene)-2-methyl-5-nitro-N-(2-(piperidin-1-yl)ethyl)benzenesulfonohydrazide. This was taken up in CH₂Cl₂ (3 mL), and then HCl in MeOH (0.5 mL, 1.25 mol L⁻¹) was added. After standing for 30 mins the solvents were removed in vacuo to leave N′-((5-bromopyrazolo[1,5-a]pyridin-3-yl)methylene)-2-methyl-5-nitro-N-(2-(piperidin-1-yl)ethyl)benzenesulfonohydrazide hydrochloride (E51) as a yellow solid (46 mg, 87%). ¹H NMR δ (400 MHz, d₆-DMSO) 10.11 (br s, 1H), 8.79 (d, J 7.30 Hz, 1H), 8.72-8.60 (m, 2H), 8.49 (dd, J 8.4, 2.4 Hz, 1H), 8.37 (s, 1H), 7.94 (s, 1H), 7.81 (d, J 8.5 Hz, 1H), 7.25 (dd, J 7.3, 2.0 Hz, 1H), 4.16 (m, 2H), 3.57 (m, 2H), 3.30 (m, 2H), 2.98 (m, 2H), 2.68 (s, 3H), 1.88-1.67 (m, 5H), 1.38 (m, 1H). LCMS (APCI⁺) 549 (MH⁺ with ⁷⁹Br, 90%), 551 (MH⁺ with ⁸¹Br, 100%). Anal. Calcd for C₂₂H₂₆BrN₆O₄S.HCl.0.5 MeOH: C, 44.90; H, 4.69; N, 13.96. Found C, 44.87; H, 4.68; N, 13.79.

### Example 52

### N′-((5-Bromopyrazolo[1,5-a]pyridin-3-yl)methylene)-2-methyl-5-nitro-N-(3-(piperidin-1-yl)propyl)benzenesulfonohydrazide hydrochloride (E52)

Step 52.1: 1-Piperidinepropanol (1.00 mL, 6.59 mmol) was added to 48% HBr (5 mL) at 0° C., stood for 10 mins, then heated under a distillation apparatus until ca. 2 mL of water distilled off. The reaction was refluxed for a further 4 h, and then the remaining HBr distilled off until the residue started to foam. After cooling to 50° C., acetone was added. The resulting precipitate was stood at 0° C. for 1 h, then filtered off, washed with acetone and dried to leave 1-(3-bromopropyl)piperidine hydrobromide as a white solid (1.47 g, 78%). ¹H NMR δ (400 MHz, d₆-DMSO) 9.09 (br s, 1H), 3.59 (t, J 6.5 Hz, 2H), 3.44 (d, J 12.1 Hz, 2H), 3.13 (m, 2H), 2.89 (m, 2H), 2.23 (m, 2H), 1.81 (m, 2H), 1.75-1.57 (m, 3H), 1.38 (m, 1H). LCMS (APCI⁺) 206 (MH⁺ with ⁷⁹Br, 100%), 208 (MH⁺ with ⁸¹Br, 90%).

Step 52.2: Reaction of E50 (40 mg, 0.091 mmol) and 1-(3-bromopropyl)piperidine hydro-bromide (39 mg, 0.14 mmol) using the conditions of Example 45 gave N′-((5-bromopyrazolo[1,5-a]pyridin-3-yl)methylene)-2-methyl-5-nitro-N-(3-(piperidin-1-yl)propyl)benzenesulfonohydrazide hydrochloride (E52) as a yellow solid (28 mg, 51%). ¹H NMR δ (400 MHz, d₆-DMSO) 9.54 (br s, 1H), 8.77 (d, J 7.3 Hz, 1H), 8.66 (d, J 2.5 Hz, 1H), 8.51-8.44 (m, 2H), 8.34 (s, 1H), 7.86 (d, J 2.2 Hz, 1H), 7.80 (d, J 8.6 Hz, 1H), 7.22 (dd, J 7.3, 2.2 Hz, 1H), 3.86 (t, J 6.9 Hz, 2H), 3.43 (m, 2H), 3.16 (m, 2H), 2.88 (m, 2H), 2.67 (s, 3H), 2.05 (m, 2H), 1.86-1.58 (m, 5H), 1.40 (m, 1H). LCMS (APCI⁺) 563 (MH⁺ with ⁷⁹Br, 95%), 565 (MH⁺ with ⁸¹Br, 100%). Anal. Calcd for C₂₃H₂₇BrN₆O₄S.HCl: C, 46.05; H, 4.70; N, 14.01. Found C, 45.73; H, 4.54; N, 13.69.

### Example 53

### N′-((5-Bromopyrazolo[1,5-a]pyridin-3-yl)methylene)-2-methyl-N-(3-morpholinopropyl)-5-nitrobenzenesulfonohydrazide hydrochloride (E53)

Reaction of E50 (50 mg, 0.11 mmol) and 1-(3-bromopropyl)morpholine hydrochloride (42 mg, 0.17 mmol) using the conditions of Example 45 gave N′-((5-bromopyrazolo[1,5-a]pyridin-3-yl)methylene)-2-methyl-N-(3-morpholinopropyl)-5-nitrobenzenesulfonohydrazide hydrochloride (E53) as a yellow solid (28 mg, 41%). ¹H NMR δ (400 MHz, d₆-DMSO) 10.01 (br s, 1H), 8.78 (d, J 7.3 Hz, 1H), 8.66 (d, J 2.4 Hz, 1H), 8.49-8.43 (m, 2H), 8.34 (s, 1H), 7.86 (s, 1H), 7.80 (d, J 8.6 Hz, 1H), 7.23 (dd, J 7.3, 2.1 Hz, 1H), 3.97 (m, 2H), 3.86 (t, J 6.8 Hz, 2H), 3.67 (m, 2H), 3.51-3.01 (m, 6H), 2.67 (s, 3H), 2.04 (m, 2H). LCMS (APCI⁺) 565 (MH⁺ with ⁷⁹Br, 100%), 567 (MH⁺ with ⁸¹Br, 100%). Anal. Calcd for C₂₂H₂₆BrN₆O₆S.HCl: C, 43.90; H, 4.35; N, 13.96. Found C, 43.60; H, 4.57; N, 13.65.

### Example 54

### 2-Methyl-N′-((5-methylpyrazolo[1,5-a]pyridin-3-yl)methylene)-5-nitro-benzenesulfonohydrazide (E54)

Reaction of 5-methylpyrazolo[1,5-a]pyridine-3-carbaldehyde (13: X=Me, Y=H) (200 mg, 1.25 mmol) using the conditions of Example 42 gave 2-methyl-N′-((5-methylpyrazolo[1,5-a]pyridin-3-yl)methylene)-5-nitrobenzenesulfonohydrazide (E54) as a yellow solid (415 mg, 89%). ¹H NMR δ (400 MHz, d₆-DMSO) 11.60 (s, 1H), 8.76 (d, J 2.5 Hz, 1H), 8.62 (d, J 7.1 Hz, 1H), 8.38 (dd, J 8.5, 2.5 Hz, 1H), 8.17 (s, 1H), 8.13 (s; 1H), 7.73 (d, J 8.5 Hz, 1H), 7.44 (m, 1H), 6.87 (dd, J 7.1, 1.9 Hz, 1H), 2.77 (s, 3H), 2.30 (s, 3H). LCMS (APCI⁺) 374 (MH⁺, 100%). Anal. Calcd for C₁₆H₁₆N₆O₄S: C, 51.47; H, 4.05; N, 18.76. Found C, 51.59; H, 4.27; N, 18.73.

### Example 55

### N′-((5-Cyanopyrazolo[1,5-a]pyridin-3-yl)methylene)-2-fluoro-N-methyl-5-nitrobenzenesulfonohydrazide (E55)

A suspension of 3-formylpyrazolo[1,5-a]pyridine-5-carbonitrile (17) (250 mg, 1.46 mmol), methylhydrazine sulfate (200 mg, 1.75 mmol) and 2,6-lutidine (1.02 mL, 8.76 mmol) in MeOH (30 mL) was stirred for 1 h until all solid had dissolved. A solution of 2-fluoro-5-nitrobenzenesulfonyl chloride (455 mg, 1.90 mmol) [A. Courtin, Helv. Chim. Acta 1982, 65 (2), 546] in CH₂Cl₂ (2 mL) was added, and the reaction stirred for a further 2 h. The precipitate was filtered off, washed with a little MeOH and dried to leave N′-((5-cyanopyrazolo[1,5-a]pyridin-3-yl)methylene)-2-fluoro-N-methyl-5-nitrobenzenesulfonohydrazide (E55) as a yellow solid (485 mg, 82%). ¹H NMR δ (400 MHz, CDCl₃) 8.93 (dd, J 5.8, 2.9 Hz, 1H), 8.53 (dd, J 7.2, 1.0 Hz, 1H), 8.49 (ddd, J 9.0, 4.0, 2.9 Hz, 1H), 8.30 (dd, J 1.8, 1.0 Hz, 1H), 8.19 (s, 1H), 7.98 (s, 1H), 7.40 (t, J 8.9 Hz, 1H), 7.01 (dd, J 7.2, 1.8 Hz, 1H), 3.46 (d, J 1.6 Hz, 3H). LCMS (APCI⁺) 403 (MH⁺, 100%). Anal. Calcd for C₁₆H₁₁FN₆O₄S: C, 47.76; H, 2.76; N, 20.89. Found C, 48.04; H, 2.97; N, 20.69.

### Example 56

### N′-((5-Cyanopyrazolo[1,5-a]pyridin-3-yl)methylene)-2-((2-(dimethylamino)ethyl)(methyl)amino)-N-methyl-5-nitrobenzenesulfonohydrazide (E56)

A solution of E55 (36 mg, 0.090 mmol) and N,N,N′-trimethylethylenediamine (46 mg, 0.45 mmol) in THF (10 mL) was stirred for 3 h. The solvent was removed in vacuo. Chromatography (eluting with CH₂Cl₂: MeOH: concentrated aqueous NH₃ 99:1:0.1 to 98:2:0.2) gave N′-((5-cyanopyrazolo[1,5-a]pyridin-3-yl)methylene)-2-((2-(dimethylamino)-ethyl)(methyl)amino)-N-methyl-5-nitrobenzenesulfonohydrazide (E56) as a yellow solid (39 mg, 91%). ¹H NMR δ (400 MHz, CDCl₃) 8.70 (d, J 2.7 Hz, 1H), 8.50 (dd, J 7.2, 0.9 Hz, 1H), 8.24 (dd, J 9.2, 2.7 Hz, 1H), 8.16 (s, 1H), 8.03 (dd, J 1.8, 0.9 Hz, 1H), 7.86 (s, 1H), 7.25 (d, J 9.2 Hz, 1H), 6.96 (dd, J 7.2, 1.8 Hz, 1H), 3.57 (t, J 6.8 Hz, 2H), 3.47 (s, 3H), 3.11 (s, 3H), 2.54 (t, J 6.8 Hz, 2H), 2.17 (s, 6H). LCMS (APCI⁺) 485 (MH⁺, 100%). Anal. Calcd for C₂₁H₂₄N₆O₄S: C, 52.06; H, 4.99; N, 23.13. Found C, 52.02; H, 5.15; N, 22.85.

### Example 57

### N′-((5-Cyanopyrazolo[1,5-a]pyridin-3-yl)methylene)-2-(2-(dimethylamino)ethylamino)-N-methyl-5-nitrobenzenesulfonohydrazide hydrochloride (E57)

Reaction of E55 (50 mg, 0.12 mmol) and N,N-dimethylethylenediamine (68 μL, 0.62 mmol) using the conditions of Example 56 gave N′-((5-cyanopyrazolo[1,5-a]pyridin-3-yl)methylene)-2-(2-(dimethylamino)ethylamino)-N-methyl-5-nitrobenzenesulfonohydrazide. This was taken up in CH₂Cl₂ (3 mL), and then HCl in MeOH (0.5 mL, 1.25 mol L⁻¹) was added. After standing for 30 mins the solvents were removed in vacuo to leave N′-((5-cyanopyrazolo[1,5-a]pyridin-3-yl)methylene)-2-(2-(dimethylamino)ethylamino)-N-methyl-5-nitrobenzenesulfonohydrazide hydrochloride (E57) as a yellow solid (49 mg, 78%). ¹H NMR δ (400 MHz, d₆-DMSO) 9.76 (br s, 1H), 8.98 (dd, J 7.2, 0.9 Hz, 1H), 8.52-8.47 (m, 3H), 8.27-8.20 (m, 2H), 7.55 (m, 1H), 7.36 (dd, J 7.2, 1.9 Hz, 1H), 7.11 (d, J 9.5 Hz, 1H), 3.80 (m, 2H), 3.38-3.20 (m, 5H), 2.82 (m, 6H). LCMS (APCI⁺) 471 (MH⁺, 100%). Anal. Calcd for C₂₀H₂₂N₆O₄S.HCl.0.5H₂O: C, 46.56; H, 4.69; N, 21.72. Found C, 46.58; H, 4.45; N, 21.48.

### Example 58

### N′-((5-Cyanopyrazolo[1,5-a]pyridin-3-yl)methylene)-N-methyl-2-(2-morpholinoethylamino)-5-nitrobenzenesulfonohydrazide hydrochloride (E58)

Reaction of E55 (50 mg, 0.12 mmol) and 4-(2-aminoethyl)morpholine (82 μL, 0.62 mmol) using the conditions of Example 57 gave N′-((5-cyanopyrazolo[1,5-a]pyridin-3-yl)methylene)-N-methyl-2-(2-morpholinoethylamino)-5-nitrobenzenesulfonohydrazide hydrochloride (E58) as a yellow solid (46 mg, 68%). ¹H NMR δ (400 MHz, d₆-DMSO) 10.26 (br s, 1H), 8.97 (dd, J 7.2, 0.9 Hz, 1H), 8.51-8.43 (m, 3H), 8.27-8.21 (m, 2H), 7.55 (m, 1H), 7.35 (dd, J 7.2, 1.9 Hz, 1H), 7.10 (m, 1H), 4.10-3.05 (m, 15H). LCMS (APCI⁺) 513 (MH⁺, 100%). Anal. Calcd for C₂₂H₂₄N₈O₆S.HCl.MeOH: C, 47.54; H, 5.03; N, 19.28. Found C, 47.27; H, 4.74; N, 19.30.

### Example 59

### N′-((5-Cyanopyrazolo[1,5-a]pyridin-3-yl)methylene)-N-methyl-2-(methyl(2-morpholinoethyl)amino)-5-nitrobenzenesulfonohydrazide hydrochloride (E59)

Reaction of E55 (50 mg, 0.12 mmol) and N-methyl-2-morpholinoethylamine (90 mg, 0.63 mmol) using the conditions of Example 57 gave N′-((5-cyanopyrazolo[1,5-a]pyridin-3-yl)-methylene)-N-methyl-2-(methyl(2-morpholinoethyl)amino)-5-nitrobenzene-sulfonohydrazide hydrochloride (E59) as a yellow solid (49 mg, 70%). ¹H NMR δ (400 MHz, d₆-DMSO) 10.08 (br s, 1H), 8.96 (dd, J 7.2, 0.8 Hz, 1H), 8.70 (d, J 2.8 Hz, 1H), 8.45-8.38 (m, 2H), 8.18 (s, 1H), 8.13 (m, 1H), 7.64 (m, 1H), 7.32 (dd, J 7.2, 1.7 Hz, 1H), 4.00-3.92 (m, 2H), 3.76-3.53 (m, 4H), 3.52-3.04 (m, 9H), 2.90 (s, 3H). LCMS (APCI⁺) 527 (MH⁺, 100%). Anal. Calcd for C₂₃H₂₆N₈O₅S.HCl.0.5H₂O: C, 48.29; H, 4.93; N, 19.59. Found C, 48.31; H, 4.91; N, 19.27.

### Example 60

### N′-((5-Cyanopyrazolo[1,5-a]pyridin-3-yl)methylene)-N-methyl-2-(methyl(2-(piperidin-1-yl)ethyl)amino)-5-nitrobenzenesulfonohydrazide hydrochloride (E60)

Reaction of E55 (50 mg, 0.12 mmol) and N-methyl-2-(1-piperidine)ethylamine (88 mg, 0.62 mmol) using the conditions of Example 57 gave N′-((5-cyanopyrazolo[1,5-a]pyridin-3-yl)methylene)-N-methyl-2-(methyl(2-(piperidin-1-yl)ethyl)amino)-5-nitrobenzenesulfonohydrazide hydrochloride (E60) as a yellow solid (67 mg, 96%). ¹H NMR δ (400 MHz, d₆-DMSO) 9.86 (br s, 1H), 8.96 (dd, J 7.2, 0.9 Hz, 1H), 8.70 (d, J 2.8 Hz, 1H), 8.42-8.37 (m, 2H), 8.19 (s, 1H), 8.13 (dd, J 1.9, 0.9 Hz, 1H), 7.65 (d, J 9.0 Hz, 1H), 7.32 (dd, J 7.2, 1.9 Hz, 1H), 3.62 (m, 2H), 3.48-3.15 (m, 7H), 2.94-2.78 (m, 5H), 1.84-1.58 (m, 5H), 1.35 (m, 1H). LCMS (APCI⁺) 525 (MH⁺, 100%). Anal. Calcd for C₂₄H₂₈N₈O₄S.HCl.0.67H₂O: C, 50.30; H, 5.34; N, 19.55. Found C, 50.34; H, 5.49; N, 19.27.

### Example 61

### N′-((5-Cyanopyrazolo[1,5-a]pyridin-3-yl)methylene)-N-methyl-2-(3-morpholinopropylamino)-5-nitrobenzenesulfonohydrazide hydrochloride (E61)

Reaction of E55 (50 mg, 0.12 mmol) and 3-morpholinopropylamine (90 mg, 0.62 mmol) using the conditions of Example 57 gave N′-((5-cyanopyrazolo[1,5-a]pyridin-3-yl)methylene)-N-methyl-2-(3-morpholinopropylamino)-5-nitrobenzenesulfonohydrazide hydrochloride (E61) as a yellow solid (68 mg, 94%). ¹H NMR δ (400 MHz, d₆-DMSO) 10.51 (br s, 1H), 8.98 (dd, J 7.2, 1.0 Hz, 1H), 8.51 (s, 1H), 8.50 (dd, J 1.9, 1.0 Hz, 1H), 8.48 (d, J 2.7 Hz, 1H), 8.25 (s, 1H), 8.21 (dd, J 9.5, 2.7 Hz, 1H), 7.49 (t, J 6.1 Hz, 1H), 7.35 (dd, J 7.2, 1.9 Hz, 1H), 7.06 (d, J 9.5 Hz, 1H), 3.94 (d, J 10.6 Hz, 2H), 3.73 (t, J 11.6 Hz, 2H), 3.51 (q, J 6.8 Hz, 2H), 3.38-3.31 (m, 5H), 3.14-2.92 (m, 4H), 1.95 (m, 2H). LCMS (APCI⁺) 527 (MH⁺, 100%). Anal. Calcd for C₂₃H₂₆N₈O₅S.HCl.1.5H₂O: C, 46.82; H, 5.12; N, 18.99. Found C, 46.81; H, 4.93; N, 18.89.

### Example 62

### N′-((5-Cyanopyrazolo[1,5-a]pyridin-3-yl)methylene)-N-methyl-2-(methyl(2-(methylamino)ethyl)amino)-5-nitrobenzenesulfonohydrazide hydrochloride (E62)

Reaction of E55 (79 mg, 0.20 mmol) and N,N′-dimethylethylenediamine (0.42 mL, 3.9 mmol) using the conditions of Example 57 gave N′-((5-cyanopyrazolo[1,5-a]pyridin-3-yl)-methylene)-N-methyl-2-(methyl(2-(methylamino)ethyl)amino)-5-nitrobenzenesulfonohydrazide hydrochloride (E62) as a yellow solid (97 mg, 97%). ¹H NMR δ (400 MHz, d₆-DMSO) 8.96 (dd, J 7.2, 0.9 Hz, 1H), 8.69 (d, J 2.8 Hz, 1H), 8.54 (br s, 2H), 8.41-8:35 (m, 2H), 8.18 (s, 1H), 8.13 (dd, J 1.8, 0.9 Hz, 1H), 7.63 (d, J 9.1 Hz, 1H), 7.32 (dd, J 7.2, 1.8 Hz, 1H), 3.50 (m, 2H), 3.40 (s, 3H), 3.09 (m, 2H), 2.89 (s, 3H), 2.52 (s, 3H). LCMS (APCI⁺) 471 (MH⁺, 100%). Anal. Calcd for C₂₀H₂₂N₈O₄S.HCl.0.5H₂O: C, 46.56; H, 4.69; N, 21.72. Found C, 46.71; H, 4.65; N, 21.72.

### Example 63

### 2-(2-(1H-Imidazol-4-yl)-ethylamino)-N′-((5-cyanopyrazolo[1,5-a]pyridin-3-yl)methylene)-N-methyl-5-nitrobenzenesulfonohydrazide hydrochloride (E63)

Reaction of E55 (50 mg, 0.12 mmol) and histamine dihydrochloride (114 mg, 0.62 mmol) using the conditions of Example 57 with the addition of NEt₃ (0.35 mL, 2.5 mmol) gave 2-(2-(1H-imidazol-4-yl)ethylamino)-N′-((5-cyanopyrazolo[1,5-a]pyridin-3-yl)methylene)-N-methyl-5-nitrobenzenesulfonohydrazide hydrochloride (E63) as a yellow solid (37 mg, 56%). ¹H NMR δ (400 MHz, d₆-DMSO) 14.21 (br s, 2H), 8.99-8.93 (m, 2H), 8.49-8.45 (m, 3H), 8.44 (dd, J 1.8, 1.0 Hz, 1H), 8.24-8.19 (m, 2H), 7.46-7.38 (m, 2H), 7.35 (dd, J 7.2, 1.8 Hz, 1H), 7.12 (d, J 9.5 Hz, 1H), 3.73 (q, J 6.9 Hz, 2H), 3.27 (s, 3H), 2.94 (t, J 6.9 Hz, 2H). LCMS (APCI⁺) 494 (MH⁺, 100%). Anal. Calcd for C₂₁H₁₉N₉O₄S.2HCl: C, 44.53; H, 3.74; N, 22.26. Found C, 44.78; H, 3.89; N, 21.95.

### Example 64

### N′-((5-Cyanopyrazolo[1,5-a]pyridin-3-yl)methylene)-N-methyl-5-nitro-2-(pyridin-2-ylmethylamino)benzenesulfonohydrazide hydrochloride (E64)

Reaction of E55 (50 mg, 0.12 mmol) and 2-(aminomethyl)pyridine (64 μL, 0.62 mmol) using the conditions of Example 57 gave N′-((5-cyanopyrazolo[1,5-a]pyridin-3-yl)methylene)-N-methyl-5-nitro-2-(pyridin-2-ylmethylamino)benzenesulfonohydrazide hydrochloride (E64) as a yellow solid (39 mg, 64%). ¹H NMR δ (400 MHz, d₆-DMSO) 8.90 (dd, J 7.2, 1.0 Hz, 1H), 8.54-8.49 (m, 2H), 8.44 (dd, J 1.8, 1.0 Hz, 1H), 8.39 (s, 1H), 8.28-8.23 (m, 2H), 8.20 (dd, J 9.4, 2.7 Hz, 1H), 7.67 (td, J 7.7, 1.6 Hz, 1H), 7.34-7.22 (m, 3H), 6.93 (d, J 9.4 Hz, 1H), 4.75 (d, J 5.4 Hz, 2H), 3.36 (s, 3H). LCMS (APCI⁺) 491 (MH⁺, 100%). Anal. Calcd for C₂₂H₁₈N₈O₄S.1.3HCl: C, 49.13; H, 3.62; N, 20.83. Found C, 49.27; H, 3.88; N, 20.89.

### Example 65

### N′-((5-Cyanopyrazolo[1,5-a]pyridin-3-yl)methylene)-N-methyl-5-nitro-2-(pyridin-3-ylmethylamino)benzenesulfonohydrazide hydrochloride (E65)

Reaction of E55 (50 mg, 0.12 mmol) and 3-(aminomethyl)pyridine (67 mg, 0.62 mmol) using the conditions of Example 57 gave N′-((5-cyanopyrazolo[1,5-a]pyridin-3-yl)methylene)-N-methyl-5-nitro-2-(pyridin-3-ylmethylamino)benzenesulfonohydrazide hydrochloride (E65) as a yellow solid (54 mg, 83%). ¹H NMR δ (400 MHz, d₆-DMSO) 8.92 (dd, J 7.2, 1.0 Hz, 1H), 8.55 (s, 1H), 8.50 (d, J 2.7 Hz, 1H), 8.48-8.42 (m, 3H), 8.25 (s, 1H), 8.14 (dd, J 9.4, 2.7 Hz, 1H), 8.00 (t, J 6.3 Hz, 1H), 7.82 (d, J 7.6 Hz, 1H), 7.35 (m, 1H), 7.29 (dd, J 7.2, 1.9 Hz, 1H), 6.89 (d, J 9.4 Hz, 1H), 4.80 (d, J 6.3 Hz, 2H), 2.54 (s, 3H). LCMS (APCI⁺) 491 (MH⁺, 100%). Anal. Calcd for C₂₂H₁₈N₈O₄S.HCl.0.33H₂O: C, 49.59; H, 3.72; N, 21.03. Found C, 49.55; H, 3.82; N, 21.05.

### Example 66

### N′-((5-Cyanopyrazolo[1,5-a]pyridin-3-yl)methylene)-N-methyl-5-nitro-2-(pyridin-4-ylmethylamino)benzenesulfonohydrazide hydrochloride (E66)

Reaction of E55 (66 mg, 0.16 mmol) and 4-(aminomethyl)pyridine (89 mg, 0.82 mmol) using the conditions of Example 57 gave N′-(5-cyanopyrazolo[1,5-a]pyridin-3-yl)methylene)-N-methyl-5-nitro-2-(pyridin-4-ylmethylamino)benzenesulfonohydrazide hydrochloride (E66) as a yellow solid (61 mg, 71%). ¹H NMR δ (400 MHz, d₆-DMSO) 8.91 (dd, J 7.2, 0.9 Hz, 1H), 8.54-8.47 (m, 5H), 8.27 (s, 1H), 8.13 (dd, J 9.4, 2.7 Hz, 1H), 8.09 (t, J 6.3 Hz, 1H), 7.54 (d, J 5.8 Hz, 2H), 7.30 (dd, J 7.2, 1.9 Hz, 1H), 6.76 (d, J 9.47 Hz, 1H), 4.94 (d, J 6.3 Hz, 2H), 3.39 (s, 3H). LCMS (APCI⁺) 491 (MH⁺, 100%). Anal. Calcd for C₂₂H₁₅N₈O₄S.HCl.0.5H₂O: C, 49.30; H, 3.76; N, 20.91. Found C, 49.05; H, 3.68; N, 20.76.

### Example 67

### N′-((5-Cyanopyrazolo[1,5-a]pyridin-3-yl)methylene)-N-methyl-2-(methyl-(pyridin-3-ylmethyl)amino)-5-nitrobenzenesulfonohydrazide hydrochloride (E67)

Reaction of E55 (50 mg, 0.12 mmol) and N-methyl-N-(3-pyridylmethyl)amine (84 mg, 0.62 mmol) using the conditions of Example 57 gave N′-((5-cyanopyrazolo[1,5-a]pyridin-3-yl)-methylene)-N-methyl-2-(methyl(pyridin-3-ylmethyl)amino)-5-nitrobenzenesulfonohydrazide hydrochloride (E67) as a yellow solid (66 mg, 99%). ¹H NMR δ (400 MHz, d₆-DMSO) 8.96 (dd, J 7.2, 1.0 Hz, 1H), 8.72-8.63 (m, 3H), 8.40 (s, 1H), 8.34 (dd, J 9.1, 2.8 Hz, 1H), 8.19 (s, 1H), 8.10 (d, J 8.2 Hz, 1H), 8.08 (dd, J 1.9, 1.0 Hz, 1H), 7.71 (dd, J 7.7, 5.4 Hz, 1H), 7.48 (d, J 9.1 Hz, 1H), 7.31 (dd, J 7.2, 1.9 Hz, 1H), 4.67 (s, 2H), 3.40 (s, 3H), 2.85 (s, 3H). LCMS (APCI⁺) 505 (MH⁺, 100%). Anal. Calcd for C₂₃H₂₀N₈O₄S.HCl.H₂O: C, 49.42; H, 4.15; N, 20.05. Found C, 49.36; H, 4.20; N, 19.97.

### Example 68

### N′-((5-Cyanopyrazolo[1,5-a]pyridin-3-yl)methylene)-2-(2-(dimethylamino)ethoxy)-N-methyl-5-nitrobenzenesulfonohydrazide hydrochloride (E68)

N,N-Dimethylethanolamine (25 μL, 0.25 mmol) was added to a suspension of NaH (10 mg, 60% in oil, 0.25 mmol) in dry THF (10 mL) at room temperature. After 30 mins, E55 (50 mg, 0.12 mmol) was added, and the reaction stirred for a further 1 h. The solvent was removed in vacuo. Chromatography (eluting with CH₂Cl₂: MeOH: concentrated aqueous NH₃ 98:2:0.2 to 97:3:0.3) gave N′-((5-cyanopyrazolo[1,5-a]pyridin-3-yl)methylene)-2-(2-(dimethylamino)ethoxy)-N-methyl-5-nitrobenzenesulfonohydrazide. This was taken up in CH₂Cl₂ (3 mL), and then HCl in MeOH (0.5 mL, 1.25 mol L⁻¹) was added. After standing for 30 mins the solvents were removed in vacuo to leave N′-((5-cyanopyrazolo[1,5-a]pyridin-3-yl)methylene)-2-(2-(dimethylamino)ethoxy)-N-methyl-5-nitrobenzenesulfonohydrazide hydrochloride (E68) as a yellow solid (37 mg, 59%). ¹H NMR δ (400 MHz, d₆-DMSO) 10.49 (br s, 1H), 8.95 (dd, J 7.2, 1.0 Hz, 1H), 8.72 (d, J 2.9 Hz, 1H), 8.55 (dd, J 9.3, 2.9 Hz, 1H), 8.39 (s, 1H), 8.16-8.12 (m, 2H), 7.56 (d, J 9.3 Hz, 1H), 7.32 (dd, J 7.2, 1.9 Hz, 1H), 4.70 (t, J 5.1 Hz, 2H), 3.57 (m, 2H), 3.49 (s, 3H), 2.89 (d, J 4.7 Hz, 6H). LCMS (APCI⁺) 472 (MH⁺, 100%). Anal. Calcd for C₂₀H₂₁N₇O₅S.HCl.1.2H₂O: C, 45.36; H, 4.64; N, 18.51. Found C, 45.34; H, 4.52; N, 18.33.

### Example 69

### N′-((5-Cyanopyrazolo[1,5-a]pyridin-3-yl)methylene)-N-methyl-2-(2-morpholinoethoxy)-5-nitrobenzenesulfonohydrazide hydrochloride (E69)

Reaction of E55 (65 mg, 0.16 mmol) and 4-(2-hydroxyethyl)morpholine (42 mg, 0.32 mmol) using the conditions of Example 68 gave N′-((5-cyanopyrazolo[1,5-a]pyridin-3-yl)methylene)-N-methyl-2-(2-morpholinoethoxy)-5-nitrobenzenesulfonohydrazide hydrochloride (E69) as a yellow solid (31 mg, 35%). ¹H NMR δ (400 MHz, d₆-DMSO) 10.44 (br s, 1H), 8.95 (d, J 7.2 Hz, 1H), 8.72 (d, J 2.9 Hz, 1H), 8.54 (m, 1H), 8.39 (s, 1H), 8.17-8.10 (m, 2H), 7.54 (d, J 9.2 Hz, 1H), 7.32 (dd, J 7.2, 1.5 Hz, 1H), 4.70 (m, 2H), 3.98 (m, 2H), 3.77-3.45 (m, 11H). LCMS (APCI⁺) 514 (MH⁺, 100%). Anal. Calcd for C₂₂H₂₃N₇O₆S.HCl.0.33H₂O: C, 47.53; H, 4.47; N, 17.64. Found C, 47.71; H, 4.41; N, 17.37.

### Example 70

### N′-((5-Cyanopyrazolo[1,5-a]pyridin-3-yl)methylene)-N-methyl-5-nitro-2-(2-(pyrrolidin-1-yl)ethoxy)benzenesulfonohydrazide hydrochloride (E70)

Reaction of E55 (50 mg, 0.12 mmol) and 1-(2-hydroxyethyl)pyrrolidine (21 mg, 0.18 mmol) using the conditions of Example 68 gave N′-((5-cyanopyrazolo[1,5-a]pyridin-3-yl)methylene)-N-methyl-5-nitro-2-(2-(pyrrolidin-1-yl)ethoxy)benzenesulfonohydrazide hydrochloride (E70) as a yellow solid (45 mg, 68%). ¹H NMR δ (400 MHz, d₆-DMSO) 10.68 (br s, 1H), 8.95 (dd, J 7.2, 1.0 Hz, 1H), 8.72 (d, J 2.9 Hz, 1H), 8.55 (dd, J 9.3, 2.9 Hz, 1H), 8.39 (s, 1H), 8.15 (s, 1H), 8.14 (dd, J 1.9, 1.0 Hz, 1H), 7.57 (d, J 9.3 Hz, 1H), 7.32 (dd, J 7.2, 1.9 Hz, 1H), 4.68 (t, J 5.1 Hz, 2H), 3.69-3.58 (m, 4H), 3.48 (s, 3H), 3.19-3.10 (m, 2H), 2.05-1.85 (m, 4H). LCMS (APCI⁺) 498 (MH⁺, 100%). Anal. Calcd for C₂₂H₂₃N₇O₅S.HCl.MeOH: C, 48.03; H, 4.95; N, 17.05. Found C, 48.06; H, 4.98; N, 17.02.

### Example 71

### N′-((5-Cyanopyrazolo[1,5-a]pyridin-3-yl)methylene)-N-methyl-5-nitro-2-(pyridin-2-ylmethoxy)benzenesulfonohydrazide hydrochloride (E71)

Reaction of E55 (50 mg, 0.12 mmol) and 2-pyridinemethanol (20 mg, 0.18 mmol) using the conditions of Example 68 gave N′-((5-cyanopyrazolo[1,5-a]pyridin-3-yl)methylene)-N-methyl-5-nitro-2-(pyridin-2-ylmethoxy)benzenesulfonohydrazide hydrochloride (E71) as a yellow solid (23 mg, 35%). ¹H NMR δ (400 MHz, d₆-DMSO) 8.93 (dd, J 7.2, 1.0 Hz, 1H), 8.75 (d, J 2.9 Hz, 1H), 8.53-8.46 (m, 2H), 8.36 (s, 1H), 8.07 (s, 1H), 8.04 (dd, J 1.8, 1.0 Hz, 1H), 7.76 (td, J 7.7, 1.8 Hz, 1H), 7.58-7.52 (m, 2H), 7.33-7.26 (m, 2H), 5.52 (s, 2H), 3.33 (s, 3H). LCMS (APCI⁺) 492 (MH⁺, 100%). Anal. Calcd for C₂₂H₁₇N₇O₆S.HCl.0.33H₂O: C, 49.49; H, 3.52; N, 18.36. Found C, 49.52; H, 3.79; N, 18.01.

### Example 72

### M-((5-Bromopyrazolo[1,5-a]pyridin-3-yl)methylene)-2-fluoro-N-methyl-5-nitrobenzenesulfonohydrazide (E72)

Reaction of 5-bromopyrazolo[1,5-a]pyridine-3-carbaldehyde (40) (149 mg, 0.66 mmol) and 2-fluoro-5-nitrobenzenesulfonyl chloride (317 mg, 1.32 mmol) using the conditions of Example 1 gave N′-((5-bromopyrazolo[1,5-a]pyridin-3-yl)methylene)-2-fluoro-N-methyl-5-nitrobenzenesulfonohydrazide (E72) as a yellow solid (194 mg, 64%). ¹FI NMR δ (400 MHz, CDCl₃) 8.98 (dd, J 5.8, 2.9 Hz, 1H), 8.48 (ddd, J 8.9, 4.0, 2.9 Hz, 1H), 8.30 (dd, J 7.3, 0.7 Hz, 1H), 8.04 (s, 1H), 7.97 (s, 1H), 7.95 (dd, J 2.2, 0.7 Hz, 1H), 7.38 (t, J 8.9 Hz, 1H), 6.97 (dd, J 7.3, 2.2 Hz, 1H), 3.45 (d, J 2.0 Hz, 3H). LCMS (APCI⁺) 456 (MH⁺ with ⁷⁹Br, 95%), 458 (MH⁺ with ⁸¹Br, 100%). Anal. Calcd for C₁₅H₁₁BrFN₅O₄S: C, 39.49; H, 2.43; N, 15.35. Found C, 39.44; H, 2.50; N, 15.29.

### Example 73

### N′-((5-Bromopyrazolo[1,5-a]pyridin-3-yl)methylene)-2-(dimethylamino)-N-methyl-5-nitrobenzenesulfonohydrazide (E73)

Reaction of E72 (50 mg, 0.11 mmol) and dimethylamine (0.55 mL, 2.0 mol L⁻¹ in MeOH, 1.1 mmol) using the conditions of Example 56 gave N′-((5-bromopyrazolo[1,5-a]pyridin-3-yl)methylene)-2-(dimethylamino)-N-methyl-5-nitrobenzenesulfonohydrazide (E73) as a yellow solid (50 mg, 94%). ¹H NMR δ (400 MHz, CDCl₃) 8.95 (d, J 2.7 Hz, 1H), 8.30-8.25 (m, 2H), 8.02 (s, 1H), 7.87 (s, 1H), 7.77 (dd, J 2.2, 0.7 Hz, 1H), 7.16 (d, J 9.1 Hz, 1H), 6.93 (dd, J 7.3, 2.2 Hz, 1H), 3.43 (s, 3H), 3.02 (s, 6H). LCMS (APCI⁺) 481 (MH⁺ with ⁷⁹Br, 100%), 483 (MH⁺ with ⁸¹Br, 90%). Anal. Calcd for C₁₇H₁₇BrN₆O₄S: C, 42.42; H, 3.56; N, 17.46. Found C, 42.71; H, 3.62; N, 17.16.

### Example 74

### N′-((5-Bromopyrazolo[1,5-a]pyridin-3-yl)methylene)-N-methyl-2-(2-morpholinoethoxy)-5-nitrobenzenesulfonohydrazide hydrochloride (E74)

Reaction of E72 (67 mg, 0.15 mmol) and 4-(2-hydroxyethyl)morpholine (29 mg, 0.22 mmol) using the conditions of Example 57 gave N′-((5-bromopyrazolo[1,5-a]pyridin-3-yl)methylene)-N-methyl-2-(2-morpholinoethoxy)-5-nitrobenzenesulfonohydrazide hydrochloride (E74) as a yellow solid (73 mg, 82%). ¹H NMR δ (400 MHz, d₆-DMSO) 10.57 (br s, 1H), 8.75 (d, J 2.9 Hz, 1H), 8.71 (dd, J 7.3, 0.7 Hz, 1H), 8.59 (dd, J 9.2, 2.9 Hz, 1H), 8.25 (s, 1H), 8.10 (s, 1H), 7.77 (m, 1H), 7.55 (d, J 9.2 Hz, 1H), 7.16 (dd, J 7.3, 2.2 Hz, 1H), 4.72 (m, 2H), 3.99 (m, 2H), 3.78-3.45 (m, 11H). LCMS (APCI⁺) 567 (MH⁺ with ⁷⁹Br, 100%), 569 (MH⁺ with ⁸¹Br, 100%). Anal. Calcd for C₂₁H₂₃BrN₆O₆S.HCl.1.5H₂O: C, 39.98; H, 4.31; N, 13.32. Found C, 40.03; H, 4.34; N, 13.07.

### Example 75

### 5-Cyano-N′-((5-cyanopyrazolo[1,5-a]pyridin-3-yl)methylene)-2-fluoro-N-methylbenzenesulfonohydrazide (E75)

Step 75.1: Reaction of 3-amino-4-fluorobenzonitrile (52: X=F, Y=CN) (500 mg, 3.67 mmol) using the conditions of Step 21.1, except with isolation of the product by extraction twice with CH₂Cl₂. The combined extracts were dried (Na₂SO₄), and the solvent removed in vacuo. Chromatography (eluting with hexanes:EtOAc 19:1 to 9:1) gave 5-cyano-2-fluorobenzenesulfonyl chloride (53: X=F, Y=CN) as a yellow oil (569 mg, 71%). ¹H NMR δ (400 MHz, CDCl₃) 8.31 (dd, J 6.2, 2.1 Hz, 1H), 8.04 (ddd, J 8.7, 4.3, 2.1 Hz, 1H), 7.51 (t, J 8.7 Hz, 1H). LCMS (APCI⁻) 200 (M-Cl+O, 100%).

Step 75.2: Reaction of 3-formylpyrazolo[1,5-a]pyridine-5-carbonitrile (17) (250 mg, 1.46 mmol) and 53 (X=F, Y=CN) (417 mg, 1.90 mmol) using the conditions of Example 31 gave 5-cyano-N′-((5-cyanopyrazolo[1,5-a]pyridin-3-yl)methylene)-2-fluoro-N-methylbenzenesulfonohydrazide (E75) as a yellow solid (300 mg, 54%). ¹H NMR δ (400 MHz, d₆-DMSO) 8.98 (dd, J 7.2, 0.9 Hz, 1H), 8.46-8.38 (m, 3H), 8.27 (ddd, J 8.7, 4.4, 2.2 Hz, 1H), 8.22 (s, 1H), 7.72 (dd, J 10.0, 8.7 Hz, 1H), 7.36 (dd, J 7.2, 1.9 Hz, 1H), 3.38 (d, J 1.3 Hz, 3H). LCMS (APCI⁺) 383 (MH⁺, 100%). Anal. Calcd for C₁₇H₁₁FN₆O₂S: C, 53.40; H, 2.90; N, 21.98. Found C, 53.39; H, 3.04; N, 22.19.

### Example 76

### 5-Cyano-N′-((5-cyanopyrazolo[1,5-a]pyridin-3-yl)methylene)-2-(dimethylamino)-N-methylbenzenesulfonohydrazide (E76)

Reaction of E75 (50 mg, 0.13 mmol) and dimethylamine (0.65 mL, 2.0 mol L⁻¹ in MeOH, 1.3 mmol) using the conditions of Example 56 gave 5-cyano-N′-((5-cyanopyrazolo[1,5-a]pyridin-3-yl)methylene)-2-(dimethylamino)-N-methylbenzenesulfonohydrazide (E76) as a yellow solid (46 mg, 87%). ¹H NMR δ (400 MHz, d₆-DMSO) 8.94 (dd, J 7.2, 1.0 Hz, 1H), 8.37 (s, 1H), 8.20 (d, J 2.1 Hz, 1H), 8.08 (s, 1H), 7.96 (dd, J 1.9, 1.0 Hz, 1H), 7.93 (dd, J 8.6, 2.1 Hz, 1H), 7.43 (d, J 8.6 Hz, 1H), 7.30 (dd, J 7.2, 1.9 Hz, 1H), 3.41 (s, 3H), 2.83 (s, 6H). LCMS (APCI⁺) 408 (MH⁺, 100%). Anal. Calcd for C₁₉H₁₇N₇O₂S: C, 56.01; H, 4.21; N, 24.06. Found C, 55.75; H, 4.32; N, 23.75.

### Example 77

### 5-Cyano-N′-((5-cyanopyrazolo[1,5-a]pyridin-3-yl)methylene)-2-((2-(dimethylamino)ethyl)(methyl)amino)-N-methylbenzenesulfonohydrazide hydro-chloride (E77)

Reaction of E75 (64 mg, 0.17 mmol) and N,N,N′-trimethylethylenediamine (86 mg, 0.84 mmol) using the conditions of Example 57 gave 5-cyano-N′-((5-cyanopyrazolo[1,5-a]pyridin-3-yl)methylene)-2-((2-(dimethylamino)ethyl)(methyl)amino)-N-methylbenzenesulfonohydrazide hydrochloride (E77) as a yellow solid (76 mg, 90%). ¹H NMR δ (400 MHz, d₆-DMSO) 10.05 (br s, 1H), 8.96 (dd, J 7.2, 0.9 Hz, 1H), 8.39 (s, 1H), 8.26 (d, J 2.0 Hz, 1H), 8.14 (s, 1H), 8.11-8.07 (m, 2H), 7.67 (d, J 8.5 Hz, 1H), 7.33 (dd, J 7.2, 1.9 Hz, 1H), 3.47-3.40 (m, 5H), 3.21 (m, 2H), 2.78 (s, 3H), 2.73 (d, J 4.6 Hz, 6H). LCMS (APCI⁺) 465 (MH⁺, 100%). Anal. Calcd for C₂₂H₂₄N₈O₂S.HCl.2H₂O: C, 49.20; H, 5.44; N, 20.86. Found C, 49.18; H, 5.23; N, 20.70.

### Example 78

### 5-Cyano-N′-((5-cyanopyrazolo[1,5-a]pyridin-3-yl)methylene)-N-methyl-2-(2-morpholinoethylamino)benzenesulfonohydrazide hydrochloride (E78)

Reaction of E75 (50 mg, 0.13 mmol) and 4-(2-aminoethyl)morpholine (86 μL, 0.66 mmol) using the conditions of Example 57 gave 5-cyano-N′-((5-cyanopyrazolo[1,5-a]pyridin-3-yl)-methylene)-N-methyl-2-(2-morpholinoethylamino)benzenesulfonohydrazide hydrochloride (E78) as a yellow solid (67 mg, 97%). ¹H NMR δ (400 MHz, d₆-DMSO) 10.31 (1×s, 1H), 8.98 (dd, J 7.2, 1.0 Hz, 1H), 8.53 (s, 1H), 8.49 (s, 1H), 8.21 (s, 1H), 8.02 (d, J 1.9 Hz, 1H), 7.83 (dd, J 8.8, 1.0 Hz, 1H), 7.35 (dd, J 7.2, 1.9 Hz, 1H), 7.31 (t, J 6.1 Hz, 1H), 7.08 (d, J 8.8 Hz, 1H), 4.07-3.90 (m, 2H), 3.88-3.62 (m, 4H), 3.50-3.05 (m, 9H). LCMS (APCI⁺) 493 (MH⁺, 100%). Anal. Calcd for C₂₃H₂₄N₈O₃S.HCl.H₂O: C, 50.50; H, 4.97; N, 20.48. Found C, 50.52; H, 4.99; N, 20.23.

### Example 79

### 5-Cyano-N′-((5-cyanopyrazolo[1,5-a]pyridin-3-yl)methylene)-N-methyl-2-(2-morpholinoethoxy)benzenesulfonohydrazide hydrochloride (E79)

Reaction of E75 (50 mg, 0.13 mmol) and 4-(2-hydroxyethyl)morpholine (26 mg, 0.20 mmol) using the conditions of Example 68 gave 5-cyano-N′-((5-cyanopyrazolo[1,5-a]-pyridin-3-yl)methylene)-N-methyl-2-(2-morpholinoethoxy)benzenesulfonohydrazide hydrochloride (E79) as a yellow solid (49 mg, 71%). ¹H NMR δ (400 MHz, d₆-DMSO) 10.44 (br s, 1H), 8.96 (dd, J 7.2, 0.9 Hz, 1H), 8.40 (s, 1H), 8.28 (d, J 2.2 Hz, 1H), 8.21-8.15 (m, 2H), 8.13 (s, 1H), 7.51 (d, J 8.9 Hz, 1H), 7.33 (dd, J 7.2, 1.9 Hz, 1H), 4.81-4.53 (m, 2H), 4.05-3.85 (m, 2H), 3.80-3.25 (m, 11H). LCMS (APCI⁺) 494 (MH⁺, 100%). Anal. Calcd for C₂₃H₂₃N₇O₄S.HCl.1.5H₂O: C, 49.59; H, 4.89; N, 17.60. Found C, 49.78; H, 4.80; N, 17.52.

### Example 80

### 2-Chloro-N′-((5-cyanopyrazolo[1,5-a]pyridin-3-yl)methylene)-N-methyl-5-nitropyridine-3-sulfonohydrazide (E80)

Reaction of 3-formylpyrazolo[1,5-a]pyridine-5-carbonitrile (17) (70 mg, 0.41 mmol) and 2-chloro-5-nitropyridine-3-sulfonyl chloride (126 mg, 0.49 mmol) [Etablissements Kuhlmann, NL 6510350 (1966)] using the conditions of Example 1 gave 2-chloro-N′-((5-cyanopyrazolo[1,5-a]pyridin-3-yl)methylene)-N-methyl-5-nitropyridine-3-sulfonohydrazide (E80) as a yellow solid (23 mg, 13%). ¹H NMR δ (400 MHz, d₆-DMSO) 9.46 (d, J 2.6 Hz, 1H), 9.02 (d, J 2.6 Hz, 1H), 8.96 (dd, J 7.2, 0.9 Hz, 1H), 8.41 (s, 1H), 8.35 (dd, J 1.9, 0.9 Hz, 1H), 8.25 (s, 1H), 7.33 (dd, J 7.2, 1.9 Hz, 1H), 3.55 (s, 3H). LCMS (APCI⁺) 420 (MH⁺ with ³⁵Cl, 100%), 422 (MH⁺ with ³⁷Clr, 30%). Anal. Calcd for C₁₅H₁₀ClN₇O₄S: C, 42.92; H, 2.40; N, 23.36. Found C, 42.71; H, 2.62; N, 23.07.

### Example 81

### N′-((5-Cyanopyrazolo[1,5-a]pyridin-3-yl)methylene)-2-((2-(dimethylamino)ethyl)(methyl)amino)-N-methyl-5-nitropyridine-3-sulfonohydrazide hydrochloride (E81)

Reaction of E80 (19 mg, 0.045 mmol) and N,N,N′-trimethylethylenediamine (29 μL, 0.22 mmol) using the conditions of Example 57 gave N′-((5-cyanopyrazolo[1,5-a]pyridin-3-yl)-methylene)-2-((2-(dimethylamino)ethyl)(methyl)amino)-N-methyl-5-nitropyridine-3-sulfonohydrazide hydrochloride (E81) as a yellow solid (18 mg, 75%). ¹H NMR δ (400 MHz, d₆-DMSO) 9.48 (br s, 1H), 9.09 (d, J 2.5 Hz, 1H), 9.01 (dd, J 7.2, 0.9 Hz, 1H), 8.68 (d, J 2.5 Hz, 1H), 8.47 (s, 1H), 8.31-8.27 (m, 2H), 7.36 (dd, J 7.2, 1.9 Hz, 1H), 4.07 (m, 2H), 3.47-3.34 (m, 8H), 2.86 (s, 6H). LCMS (APCI⁺) 486 (MH⁺, 100%). Anal. Calcd for C₂₀H₂₃H₆O₄S.HCl.1.5H₂O: C, 43.76; H, 4.96; N, 22.96. Found C, 43.78; H, 4.96; N, 22.58.

### Example 82

### N′-((5-Cyanopyrazolo[1,5-a]pyridin-3-yl)methylene)-N-methyl-3-nitro-benzohydrazide (E82)

Reaction of 3-formylpyrazolo[1,5-a]pyridine-5-carbonitrile (17) (30 mg, 0.18 mmol) and 3-nitrobenzoyl chloride (65 mg, 0.35 mmol) using the conditions of Example 1 gave N′-((5-cyanopyrazolo[1,5-a]pyridin-3-yl)methylene)-N-methyl-3-nitrobenzohydrazide (E82) as a yellow solid (55 mg, 90%). ¹H NMR δ (400 MHz, d₆-DMSO) 8.93 (dd, J 7.2, 0.9 Hz, 1H), 8.46-8.41 (m, 2H), 8.37 (ddd, J 8.2, 2.4, 1.1 Hz, 1H), 8.30 (s, 1H), 8.06 (dt, J 7.7, 1.3 Hz, 1H), 7.85 (m, 1H), 7.59 (s, 1H), 7.26 (dd, J 7.2, 1.9 Hz, 1H), 3.54 (s, 3H). LCMS (APCI⁺) 349 (MH⁺, 100%). Anal. Calcd for C₁₇H₁₂N₆O₃: C, 58.62; H, 3.47; N, 24.13. Found C, 58.46; H, 3.54; N, 24.38.

### Example 83

### N′-((5-Cyanopyrazolo[1,5-a]pyridin-3-yl)methylene)-N,2-dimethyl-5-nitrobenzohydrazide (E83)

Reaction of 17 (30 mg, 0.18 mmol) and 2-methyl-5-nitrobenzoyl chloride (70 mg, 0.35 mmol) using the conditions of Example 1 gave N′-((5-cyanopyrazolo[1,5-a]pyridin-3-yl)methylene)-N,2-dimethyl-5-nitrobenzohydrazide (E83) as a yellow solid (40 mg, 63%). ¹H NMR δ (400 MHz, d₆-DMSO) 8.91 (dd, J 7.2, 0.9 Hz, 1H), 8.42 (s, 1H), 8.28 (s, 1H), 8.24 (dd, J 8.4, 2.5 Hz, 1H), 8.19 (d, J 2.5 Hz, 1H), 7.68 (d, J 8.4 Hz, 1H), 7.23 (dd, J 7.2, 1.9 Hz, 1H), 7.09 (dd, J 1.9, 0.9 Hz, 1H), 3.54 (s, 3H), 2.30 (s, 3H). LCMS (APCI⁺) 363 (MH⁺, 100%). Anal. Calcd for C₁₈H₁₄N₆O₃: C, 59.67; H, 3.89; N, 23.19. Found C, 59.46; H, 3.94; N, 23.11.

### Example 84

### 3-((2-Methyl-2-(2-methyl-5-nitrobenzyl)hydrazono)methyl)pyrazolo[1,5-a]pyridine-5-carbonitrile (E84)

Step 84.1: A solution of 2-(chloromethyl)-1-methyl-4-nitrobenzene (63: Z=2-Me, 5-NO₂) (200 mg, 1.08 mmol) [D. R. Maulding et al., J. Org. Chem. 1983, 48 (17), 2938] and methyl-hydrazine (0.28 mL, 5.3 mmol) in EtOH (6 mL) was refluxed for 1 h. The solvent was removed in vacuo, then taken up in CH₂Cl₂, washed with 1M aqueous NaOH, dried (Na₂SO₄) and the solvent removed in vacuo to leave 1-methyl-1-(2-methyl-5-nitrobenzyl)hydrazine (64: Z=2-Me, 5-NO₂) as a yellow oil (210 mg, 100%). ¹H NMR δ (400 MHz, CDCl₃) 8.20 (d, J 2.5 Hz, 1H), 8.04 (dd, J 8.3, 2.5 Hz, 1H), 7.31 (d, J 8.3 Hz, 1H), 3.65 (s, 2H), 3.00 (br s, 2H), 2.57 (s, 3H), 2.47 (s, 3H). LCMS (APCI⁺) 196 (MH⁺, 100%).

Step 84.2: A suspension of 17 (30 mg, 0.18 mmol) and 64 (Z=2-Me, 5-NO₂) (35 mg, 0.18 mmol) was stirred in MeOH (10 mL) for 3 days. The precipitate was filtered off and dried to leave 3-((2-methyl-2-(2-methyl-5-nitrobenzyl)hydrazono)methyl)pyrazolo[1,5-a]pyridine-5-carbonitrile (E84) as a yellow solid (40 mg, 66%). ¹H NMR δ (400 MHz, CDCl₃) 8.45 (dd, J 7.2, 0.9 Hz, 1H), 8.27 (dd, J 1.8, 0.9 Hz, 1H), 8.18 (d, J 2.4 Hz, 1H), 8.12 (dd, J 8.3, 2.4 Hz, 1H), 8.07 (s, 1H), 7.49 (s, 1H), 7.41 (d, J 8.3 Hz, 1H), 6.86 (dd, J 7.2, 1.8 Hz, 1H), 4.50 (s, 2H), 2.95 (s, 3H), 2.51 (s, 3H). LCMS (APCI⁺) 349 (MH⁺, 100%). Anal. Calcd for C₁₈H₁₆N₆O₂: C, 62.06; H, 4.63; N, 24.12. Found C, 61.76; H, 4.57; N, 24.06.

### Example 85

### 3-(1-(2-Methyl-5-nitrophenylsulfonyl)-1H-pyrazol-3-yl)pyrazolo[1,5-a]-pyridine (E85)

Step 85.1: A solution of N,N-dimethylformamide dimethyl acetal (1.60 mL, 12.0 mmol) and 1-(pyrazolo[1,5-a]pyridin-3-yl)ethanone (69: X=H) (380 mg, 2.38 mmol) [T. Irikura, DE 2546196 (1976)] in dry DMF (10 mL) was heated to 90° C. for 2 days. The solution was then diluted with water and extracted three times with EtOAc. The combined organic phases were washed three times with water then with brine, dried (Na₂SO₄) and the solvent removed in vacuo. The solid residue was taken up in EtOH (15 mL), then N₂H₄.H₂O (0.58 mL, 11.9 mmol) added, and the solution refluxed for 2 h. After removal of the solvent in vacuo, chromatography (eluting with hexanes:EtOAc 3:1 to 2:1 to 1:1) gave 3-(1H-pyrazol-3-yl)pyrazolo[1,5-a]pyridine (70: X=H) as a pale yellow oil (269 mg, 62%). ¹H NMR δ (400 MHz, CDCl₃) 8.48 (dt, J 6.9, 1.0 Hz, 1H), 8.22 (s, 1H), 8.02 (d, J 8.9 Hz, 1H), 7.65 (d, J 2.3 Hz, 1H), 7.18 (ddd, J 8.9, 6.9, 1.1 Hz, 1H), 6.80 (td, J 6.9, 1.3 Hz, 1H), 6.56 (d, J 2.3 Hz, 1H). LCMS (APCI⁺) 185 (MH⁺, 100%).

Step 85.2: A solution of 70 (X=H) (50 mg, 0.27 mmol), 2-methyl-5-nitrobenzenesulfonyl chloride (70 mg, 0.30 mmol) and NEt₃ (57 μL, 0.41 mmol) in CH₂Cl₂ (5 mL) was stirred at room temperature for 3 days. The solution was diluted with water, the layers separated, the aqueous phase extracted with CH₂Cl₂, then the combined organic layers were dried (Na₂SO₄) and the solvent removed in vacuo. Chromatography (eluting with hexanes:EtOAc 4:1 to 3:1 to 2:1) gave 3-(1-(2-methyl-5-nitrophenylsulfonyl)-1H-pyrazol-3-yl)pyrazolo[1,5-a]pyridine (E85) as a yellow solid (45 mg, 43%). ¹H NMR δ (400 MHz, CDCl₃) 9.00 (d, J 2.4 Hz, 1H), 8.45 (dt, J 6.9, 1.0 Hz, 1H), 8.33 (dd, J 8.4, 2.4 Hz, 1H), 8.21 (d, J 2.8 Hz, 1H), 8.20 (s, 1H), 8.10 (dt, J 8.9, 1.3 Hz, 1H), 7.50 (d, J 8.4 Hz, 1H), 7.29 (ddd, J 8.9, 6.9, 1.1 Hz, 1H), 6.87 (td, J 6.9, 1.3 Hz, 1H), 6.72 (d, J 2.8 Hz, 1H), 2.86 (s, 3H). LCMS (APCI⁺) 384 (MH⁺, 100%). Anal. Calcd for C₁₇H₁₃N₅O₄S.0.2 EtOAc: C, 53.32; H, 3.67; N, 17.46. Found C, 53.60; H, 3.73; N, 17.48.

### Example 86

### 3-(1-(3-Nitrophenylsulfonyl)-1H-pyrazol-3-yl)pyrazolo[1,5-a]pyridine (E86)

Reaction of 70 (X=H) (108 mg, 0.59 mmol) and 3-nitrobenzenesulfonyl chloride (156 mg, 0.70 mmol) using the conditions of Step 85.2 gave 3-(1-(3-nitrophenylsulfonyl)-1H-pyrazol-3-yl)pyrazolo[1,5-a]pyridine (E86) as a yellow solid (132 mg, 61%). ¹H NMR δ (400 MHz, CDCl₃) 8.97 (t, J 2.0 Hz, 1H), 8.37-8.49 (m, 3H), 8.18-8.23 (m, 2H), 8.15 (d, J 2.9 Hz, 1H), 7.76 (t, J 8.1 Hz, 1H), 7.35 (ddd, J 8.9, 6.9, 1.1 Hz, 1H), 6.89 (td, J 6.9, 1.3 Hz, 1H), 6.70 (d, J 2.9 Hz, 1H). LCMS (APCI⁺) 370 (MH⁺, 100%). Anal. Calcd for C₁₆H₁₁N₅O₄S: C, 52.03; H, 3.00; N, 18.96. Found C, 52.19; H, 3.05; N, 18.94.

### Example 87

### 3-(3-(Pyrazolo[1,5-a]pyridin-3-yl)-1H-pyrazol-1-ylsulfonyl)benzonitrile (E87)

Reaction of 70 (X=H) (108 mg, 0.59 mmol) and 3-cyanobenzenesulfonyl chloride (142 mg, 0.70 mmol) using the conditions of Step 85.2 gave 3-(3-(pyrazolo[1,5-a]pyridin-3-yl)-1H-pyrazol-1-ylsulfonyl)benzonitrile (E87) as a yellow solid (146 mg, 71%). ¹H NMR δ (400 MHz, CDCl₃) 8.48 (d, J 6.9 Hz, 1H), 8.38 (t, J 1.5 Hz, 1H), 8.29 (dt, J 7.9, 1.5 Hz, 1H), 8.20 (s, 1H), 8.17 (d, J 8.9 Hz, 1H), 8.13 (d, J 2.8 Hz, 1H), 7.89 (dt, J 7.9, 1.3 Hz, 1H), 7.68 (t, J 7.9 Hz, 1H), 7.34 (ddd, J 8.9, 6.9, 1.0 Hz, 1H), 6.89 (td, J 6.9, 1.3 Hz, 1H), 6.70 (d, J 2.8 Hz, 1H). LCMS (APCI⁺) 350 (MH⁺, 100%). Anal. Calcd for C₁₇H₁₁N₅O₂S: C, 58.44; H, 3.17; N, 20.05. Found C, 58.33; H, 3.26; N, 20.02.

### Example 88

### 5-Bromo-3-(1-(2-methyl-5-nitrophenylsulfonyl)-1H-pyrazol-3-yl)pyrazolo[1,6-a]pyridine (E88)

Step 88.1: Reaction of tert-butyl pyridin-4-ylcarbamate (1: X=NHCO₂^{t}Bu) (0.99 g, 5.1 mmol) and 3-butyn-2-one (0.48 mL, 6.13 mmol) using the conditions of Step 2.1 gave tert-butyl 3-acetylpyrazolo[1,5-a]pyridin-5-ylcarbamate (66) as a yellow solid (81 mg, 6%). ¹H NMR δ (400 MHz, CDCl₃) 8.41 (d, J 7.6 Hz, 1H), 8.26 (s, 1H), 8.08 (d, J 2.3 Hz, 1H), 7.58 (m, 1H), 6.96 (s, 1H), 2.71 (s, 3H), 1.55 (s, 9H). LCMS (APCI⁺) 276 (MH⁺, 100%).

Step 88.2: Reaction of 66 (81 mg, 0.29 mmol) using the conditions of Step 12.2 gave the trifluoroacetate salt of 1-(5-aminopyrazolo[1,5-a]pyridin-3-yl)ethanone (67) as a brown solid (86 mg, 100%). ¹H NMR δ (400 MHz, d₆-DMSO) 8.33 (d, J 7.4 Hz, 1H), 8.22 (s, 1H), 7.51 (d, J 2.6 Hz, 1H), 6.46 (dd, J 7.4, 2.6 Hz, 1H), 2.52 (s, 3H). LCMS (APCI⁺) 176 (MH⁺, 100%).

Step 88.3: Reaction of 67 (86 mg, 0.29 mmol) using the conditions of Step 14.1 gave 1-(5-bromopyrazolo[1,5-a]pyridin-3-yl)ethanone (68) as a white solid (30 mg, 43%). ¹H NMR δ (400 MHz, CDCl₃) 8.61 (dd, J 2.2, 0.8 Hz, 1H), 8.37 (dd, J 7.3, 0.8 Hz, 1H), 8.32 (s, 1H), 7.10 (dd, J 7.3, 2.2 Hz, 1H), 2.55 (s, 3H). LCMS (APCI⁺) 239 (MH⁺ with ⁷⁹Br, 100%), 241 (MH⁺ with ⁸¹Br, 80%).

Step 88.4: Reaction of 68 (30 mg, 0.13 mmol) using the conditions of Step 85.1 gave 5-bromo-3-(1H-pyrazol-3-yl)pyrazolo[1,5-a]pyridine (70: X=Br) as a yellow solid (27 mg, 82%). ¹H NMR δ (400 MHz, CDCl₃) 8.28-8.34 (m, 2H), 8.20 (s, 1H), 7.65 (d, J 2.4 Hz, 1H), 6.88 (dd, J 7.4, 2.0 Hz, 1H), 6.57 (d, J 2.4 Hz, 1H). LCMS (APCI⁺) 263 (MH⁺ with ⁷⁹Br, 80%), 265 (MH⁺ with ⁸¹Br, 100%).

Step 88.5: Reaction of 70 (X=Br) (27 mg, 0.10 mmol) and 2-methyl-5-nitrobenzenesulfonyl chloride (36 mg, 0.15 mmol) using the conditions of Step 85.2 gave 5-bromo-3-(1-(2-methyl-5-nitrophenylsulfonyl)-1H-pyrazol-3-yl)pyrazolo[1,5-a]pyridine (E88) as a yellow solid (36 mg, 77%). ¹H NMR δ (400 MHz, CDCl₃) 8.99 (d, J 2.4 Hz, 1H), 8.36 (dd, J 8.4, 2.4 Hz, 1H), 8.31 (dd, J 7.3, 0.7 Hz, 1H), 8.22 (d, J 2.8 Hz, 1H), 8.19 (dd, J 2.1, 0.7 Hz, 1H), 8.17 (s, 1H), 7.55 (d, J 8.4 Hz, 1H), 6.93 (dd, J 7.3, 2.1 Hz, 1H), 6.70 (d, J 2.8 Hz, 1H), 2.90 (s, 3H). LCMS (APCI⁺) 462 (MH⁺ with ⁷⁹Br, 100%), 464 (MH⁺ with ⁸¹Br, 75%). Anal. Calcd for C₁₇H₁₂BrN₆O₄S.0.2 EtOAc: C, 44.55; H, 2.86; N, 14.59. Found C, 44.66; H, 2.80; N, 14.84.

### Example 89

### 4-(5-Bromopyrazolo[1,5-a]pyridin-3-0)-2-(3-nitrophenylsulfinyl)thiazole (E89)

Step 89.1: Bromine (805 mg, 5.04 mmol) in AcOH (5 mL) was added to a suspension of 1-(5-bromopyrazolo[1,5-a]pyridin-3-yl)ethanone (69: X=Br) (1.21 g, 5.04 mmol) in 30% HBr/AcOH (15 mL). The orange slurry was stirred at room temperature overnight to give a cream coloured precipitate, which was diluted with cold Et₂O (10 mL). The precipitate was collected by filtration, washed with ice cold EtOH (5 mL) and Et₂O (5 mL) to give 2-bromo-1-(5-bromopyrazolo[1,5-a]pyridin-3-yl)ethanone (72: X=Br) as a cream solid (1.52 g, 95%). ¹H NMR δ (400 MHz, CDCl₃) 8.60 (d, J 1.9 Hz, 1H), 8.40 (m, 2H), 7.16 (dd, J 7.2, 2.1 Hz, 1H), 4.28 (s, 2H). LCMS (APCI⁺) 317 (MH⁺ with ⁷⁹Br₂, 80%), 319 (MH⁺ with ⁷⁸Br⁸¹Br, 100%), 321 (MH⁺ with ⁸¹Br₂, 60%).

Step 89.2: Ammonium dithiocarbamate (208 mg, 1.89 mmol) and 72 (X=Br) (200 mg, 0.63 mmol) in anhydrous MeOH (10 mL) was stirred for 10 mins. The resulting cream precipitate was collected by filtration, resuspended in acetic acid (10 mL) and refluxed for 1 hr. The reaction was cooled to room temperature and diluted with ice cold water. The resulting green precipitate was collected by filtration, redissolved in (CH₂Cl₂:MeOH 95:5, 100 mL) and dried (Na₂SO₄). The pale green solution was concentrated in vacuo to give 4-(5-bromopyrazolo[1,5-a]pyridin-3-yl)thiazole-2 (3H)-thione (73: X=Br) (136 mg, 70%) that is stored in the freezer. ¹H NMR δ (400 MHz, d₆-DMSO) 13.59 (br s, 1H), 8.74 (d, J 7.3 Hz, 1H), 8.51 (s, 1H), 8.22 (d, J 1.8 Hz, 1H), 7.27 (s, 1H), 7.19 (dd, J 7.3, 2.1 Hz, 1H). LCMS (APCI⁺) 312 (MH⁺ with ⁷⁹Br, 100%), 314 (MH⁺ with ⁸¹Br, 100%).

Step 89.3: 73 (X=Br) (50 mg, 0.149 mmol), Cu(OAc)₂, (29 mg, 0.149 mmol), 1,10-phenanthroline (57.6 mg, 0.298 mmol) and 3-nitrobenzeneboronic acid (107 mg, 0.640 mmol) in 1,2-dichloroethane (10 mL) was stirred vigorously at room temperature for 30 mins. The red solution was refluxed for 48 h to give a blue precipitate which was concentrated in vacuo. Chromatography (eluting with CH₂Cl₂) gave 4-(5-bromopyrazolo[1,5-a]pyridin-3-yl)-2-(3-nitrophenylthio)thiazole (74: X=Br, Z=3-NO₂) (46 mg, 72%) as a yellow solid. ¹H NMR δ (400 MHz, CDCl₃) 8.54 (t, J 1.96 Hz, 1H), 8.29 (dd, J 7.3, 0.7 Hz, 1H), 8.28 (ddd, J 8.2, 2.2, 1.0 Hz, 1H), 8.23 (s, 1H), 8.19 (dd, J 2.1, 0.7 Hz, 1H), 7.98 (ddd, J 7.8, 1.7, 1.0 Hz, 1H), 7.64 (t, J 8.0 Hz, 1H), 7.30 (s, 1H), 6.87 (dd, J 7.3, 2.1 Hz, 1H). LCMS (APCI⁺) 433 (MH⁺ with ⁷⁹Br, 100%), 435 (MH⁺ with ⁸¹Br, 85%).

Step 89.4: 74 (X=Br, Z=3-NO₂) (22 mg, 0.05 mmol) was added to a stirred solution of Oxone® (8 mg, 0.25 mmol) in (MeOH:water 1:1, 6 mL). The reaction was stirred at room temperature for 3 days and the methanol was removed in vacuo. The aqueous solution was diluted with water (20 mL), extracted twice with CH₂Cl₂ (20 mL). The combined extracts were dried (Na₂SO₄) and the solvent removed in vacuo. Chromatography (gradient elution with CH₂Cl₂:EtOAc 1:0 to 9:1) gave 4-(5-bromopyrazolo[1,5-a]pyridin-3-yl)-2-(3-nitrophenylsulfinyl)thiazole (E89) as a lime green solid (15 mg, 66%). ¹H NMR δ (400 MHz, d₅-DMSO) 8.71 (dd, J 7.3, 0.7 Hz, 1H), 8.68 (t, J 1.9 Hz, 1H), 8.58 (s, 1H), 8.46 (ddd, J 8.2, 2.3, 1.0 Hz, 1H), 8.36 (ddd, J 7.8, 1.6, 1.0 Hz, 1H), 8.34 (s, 1H), 8.26 (dd, J 2.2, 0.7 Hz, 1H), 7.96 (t, J 8.0 Hz, 1H), 7.13 (dd, J 7.3, 2.2 Hz, 1H). LCMS (APCI⁺) 449 (MH⁺ with ⁷⁹Br, 100%), 451 (MH⁺ with ⁸¹Br, 85%).

### Example 90

### 4-(5-Bromopyrazolo[1,5-a]pyridin-3-yl)-2-(3-nitrophenylsulfonyl)thiazole (E90)

A solution of magnesium monoperoxyphthalate hexahydrate (285 mg, 5 mmol) in EtOH (10 mL) was added dropwise to E89 (50 mg, 0.115 mmol) in CH₂Cl₂ (2 mL) and stirred at room temperature for 2 hr. The reaction was diluted with 5% NaHCO₃ (50 mL) and extracted three times with EtOAc (50 mL). The combined extracts were washed with dried (Na₂SO₄) and the solvent removed in vacuo. Chromatography (gradient elution with CH₂Cl₂: EtOAc 1:0 to 7:3) gave 4-(5-bromopyrazolo[1,5-a]pyridin-3-yl)-2-(3-nitrophenylsulfonyl)thiazole (E90) as a brown solid (7 mg, 14%). ¹H NMR (400 MHz, CDCl₃) 1H NMR 9.02 (t, J 1.8 Hz, 1H), 8.55 (dd, J 8.0, 2.0 Hz, 2H), 8.31 (dd, J 7.3, 0.7 Hz, 1H), 8.24 (s, 1H), 8.15 (dd, J 2.1, 0.7 Hz, 1H), 7.87 (t, J 8.0 Hz, 1H), 7.63 (s, 1H), 6.93 (dd, J 7.3, 2.1 Hz, 1H). LCMS (APCI⁺) 463.8 (MH⁺ with ⁷⁹Br, 95%), 465.8 (MH⁺ with ⁸¹Br, 100%).

### Example 91

### 2-(5-Bromopyrazolo[1,5-a]pyridin-3-yl)-5-(2-methyl-5-nitrophenylthio)-1,3,4-oxadiazole (E91)

Step 91.1: A solution of ethyl 5-bromopyrazolo[1,5-a]pyridine-3-carboxylate (38: X=Br) (300 mg, 1.12 mmol) and N₂H₄.H₂O (1.08 mL, 22.3 mmol) in EtOH (5 mL) was refluxed for 18 h. After cooling to room temperature, the reaction mixture was diluted with water, the precipitate was filtered off, washed with water and dried to leave 5-bromopyrazolo[1,5-a]-pyridine-3-carbohydrazide (77: X=Br) as a white solid (196 mg, 69%). ¹H NMR δ (400 MHz, d₆-DMSO) 9.52 (br s, 1H), 8.74 (dd, J 7.3, 0.7 Hz, 1H), 8.52 (s, 1H), 8.37 (d, J 2.2 Hz, 1H), 7.20 (dd, J 7.3, 2.2 Hz, 1H), 4.39 (br s, 2H). LCMS (APCI⁺) 255 (MH⁺ with ⁷⁹Br, 100%), 257 (MH⁺ with ⁸¹Br, 95%).

Step 91.2: A suspension of 77 (X=Br) (194 mg, 0.76 mmol), CS₂ (69 μL, 1.15 mmol) and KOH (43 mg, 0.77 mmol) in EtOH (5 mL) was refluxed for 28 h. The solvent was removed in vacuo. Water was added to the residue, it was acidified to pH 1 with 1M aqueous HCl, and then the solid filtered off, washed with water and dried to leave 5-(5-bromopyrazolo[1,5-a]pyridin-3-yl)-1,3,4-oxadiazole-2 (3H)-thione (78: X=Br) as a white solid (187 mg, 83%). ¹H NMR δ (400 MHz, d₆-DMSO) 8.89 (d, J 7.3 Hz, 1H), 8.62 (s, 1H), 8.12 (d, J 2.1 Hz, 1H), 7.36 (dd, J 7.3, 2.1 Hz, 1H). LCMS (APCI⁻) 295 (M-H⁺ with ⁷⁹Br, 100%), 297 (M-H⁺ with ⁸¹Br, 90%).

Step 91.3: Reaction of 78 (X=Br) (175 mg, 0.59 mmol) and 2-methyl-5-nitrophenyl-boronic acid (426 mg, 2.35 mmol) using the conditions of Step 89.3 gave 2-(5-bromo-pyrazolo[1,5-a]pyridin-3-yl)-5-(2-methyl-5-nitrophenylthio)-1,3,4-oxadiazole (E91) as a pale yellow solid (141 mg, 55%). ¹H NMR δ (400 MHz, CDCl₃) 8.58 (d, J 2.4 Hz, 1H), 8.40 (dd, J 7.3, 0.6 Hz, 1H), 8.36 (s, 1H), 8.33 (dd, J 2.1, 0.6 Hz, 1H), 8.23 (dd, J 8.4, 2.4 Hz, 1H), 7.54 (d, J 8.4 Hz, 1H), 7.08 (dd, J 7.3, 2.1 Hz, 1H), 2.66 (s, 3H). LCMS (APCI⁺) 432 (MH⁺ with ⁷⁹Br, 95%), 434 (MH⁺ with ⁸¹Br, 100%). Anal. Calcd for C₁₆H₁₀BrN₅O₃S: C, 44.46; H, 2.33; N, 16.20. Found C, 44.76; H, 2.52; N, 16.22.

### Example 92

### 2-(5-Bromopyrazolo[1,5-a]pyridin-3-yl)-5-(2-methyl-5-nitrophenyl-sulfinyl)-1,3,4-oxadiazole (E92)

H₂O₂ (39 μL, 70%, 0.80 mmol) was added to a solution of trifluoroacetic anhydride (0.11 mL, 0.79 mmol) in CH₂Cl₂ (5 mL) at 0° C. After 5 mins, the solution was warmed to room temperature for 15 mins. This was added to a solution of E91 (70 mg, 0.16 mmol) in CH₂Cl₂ (10 mL) at 0° C. After 1 h, 5% aqueous Na₂SO₃ was added, and the layers were separated. The aqueous layer was extracted with CH₂Cl₂, the combined extracts were dried (Na₂SO₄) and the solvent removed in vacuo. Chromatography (eluting with hexanes:EtOAc 3:1 to 2:1) gave 2-(5-bromopyrazolo[1,5-a]pyridin-3-yl)-5-(2-methyl-5-nitrophenylsulfinyl)-1,3,4-oxadiazole (E92) as a white solid (36 mg, 49%). ¹H NMR δ (400 MHz, CDCl₃) 9.10 (d, J 2.4 Hz, 1H), 8.43 (s, 1H), 8.41 (dd, J 7.3, 0.7 Hz, 1H), 8.37-8.32 (m, 2H), 7.49 (d, J 8.3 Hz, 1H), 7.12 (dd, J 7.3, 2.1 Hz, 1H), 2.57 (s, 3H). LCMS (APCI⁺) 448 (MH⁺ with ⁷⁹Br, 100%), 450 (MH⁺ with ⁸¹Br, 100%). Anal. Calcd for C₁₆H₁₀BrN₆O₄S.0.1 EtOAc: C, 43.10; H, 2.38; N, 15.32. Found C, 43.30; H, 2.44; N, 15.28.

### Example 93

### 2-(5-Bromo-2-methylphenylsulfonyl)-5-(5-bromopyrazolo[1,5-a]pyridin-3-yl)-1,3,4-thiadiazole (E93)

Step 93.1: Reaction of ethyl 5-bromopyrazolo[1,5-a]pyridine-3-carboxylate (38: X=Br) (583 mg, 2.17 mmol) using the conditions of Step 2.2 gave 5-bromopyrazolo[1,5-a]pyridine-3-carboxylic acid (5: X=Br) as a white solid (504 mg, 97%). ¹H NMR δ (400 MHz, d₆-DMSO) 12.61 (s, 1H), 8.82 (d, J 7.3 Hz, 1H), 8.42 (s, 1H), 8.22 (d, J 2.2 Hz, 1H), 7.29 (dd, J 7.3, 2.2 Hz, 1H). LCMS (APCI⁻) 239 (M-H⁺ with ⁷⁹Br, 90%), 241 (M-H⁺ with ⁸¹Br, 100%).

Step 93.2: 5 (X=Br) (100 mg, 0.41 mmol) was refluxed in SOCl₂ (3 mL) for 1 h. The solvent was removed in vacuo. The residue was taken up in CH₂Cl₂ (2 mL) and added dropwise to a solution of thiosemicarbazide (38 mg, 0.42 mmol) in pyridine (5 mL) at 0° C. over 5 mins. After 1 h, the solvents were removed in vacuo. The residue was taken up in concentrated H₂SO₄ (3 mL) and heated to 50° C. for 1 h. Ice was added to the solution, and then it was basified to pH 6 with 6M aqueous NaOH and stood for 1 h. The precipitate was filtered off, washed with water and dried to leave 5-(5-bromopyrazolo[1,5-a]pyridin-3-yl)-1,3,4-thiadiazol-2-amine (81: X=Br) as a pale yellow solid (120 mg, 98%). ¹H NMR δ (400 MHz, d₆-DMSO) 8.77 (dd, J 7.3, 0.7 Hz, 1H), 8.42 (s, 1H), 8.32 (dd, J 2.2, 0.7 Hz, 1H), 7.25 (br s, 2H), 7.22 (dd, J 7.3, 2.2 Hz, 1H). LCMS (APCI⁺) 296 (MH⁺ with ⁷⁹Br, 95%), 298 (MH⁺ with ⁸¹Br, 100%).

Step 93.3: ^{t}BuONO (83 μL, 0.63 mmol) was added to a suspension of 81 (X=Br) (93 mg, 0.31 mmol) and CuCl₂.2H₂O (64 mg, 0.38 mmol) in dry MeCN (5 mL). The reaction mixture was stirred at room temperature for 18 h, then diluted with water and extracted twice with CH₂Cl₂. The combined extracts were dried (Na₂SO₄) and the solvents removed in vacuo. Chromatography (eluting with CH₂Cl₂: MeOH 99.75:0.25) gave 2-(5-bromopyrazolo[1,5-a]pyridin-3-yl)-5-chloro-1,3,4-thiadiazole (82: X=Br) as a yellow solid (30 mg, 30%). ¹H NMR δ (400 MHz, CDCl₃) 8.59 (dd, J 2.1, 0.8 Hz, 1H), 8.39 (dd, J 7.3, 0.8 Hz, 1H), 8.23 (s, 1H), 7.09 (dd, J 7.3, 2.1 Hz, 1H). LCMS (APCI⁺) 315 (MH⁺ with ⁷⁹Br³⁵Cl, 70%), 317 (MH⁺ with ⁷⁹Br³⁷Cl and ⁸¹Br³⁸Cl, 100%), 517 (MH⁺ with ⁸¹Br³⁷Cl, 30%).

Step 93.4: A solution of 82 (X=Br) (30 mg, 0.095 mmol) and sodium 5-bromo-2-methyl-benzenesulfinate (122 mg, 0.47 mmol) [H.-W. Kleemann et al., DE 19832429 (2000)] in DMSO (1 mL) was heated to 120° C. for 18 h. The reaction mixture was then diluted with CH₂Cl₂, washed twice with water, dried (Na₂SO₄) and the solvent removed in vacuo. Chromatography (eluting with hexanes:EtOAc 9:1 to 85:15 to 4:1) gave 2-(5-bromo-2-methylphenylsulfonyl)-5-(5-bromopyrazolo[1,5-a]pyridin-3-yl)-1,3,4-thiadiazole (E93) as a yellow solid (7 mg, 14%). ¹H NMR δ (400 MHz, CDCl₃) 8.63 (dd, J 2.1, 0.7 Hz, 1H), 8.41 (dd, J 7.3, 0.7 Hz, 1H), 8.36 (d, J 2.1 Hz, 1H), 8.32 (s, 1H), 7.69 (dd, J 8.2, 2.1 Hz, 1H), 7.24 (d, J 8.2 Hz, 1H), 7.13 (dd, J 7.3, 2.1 Hz, 1H), 2.73 (s, 3H). LCMS (APCI⁺) 513 (MH⁺ with ⁷⁹Br₂, 50%), 515 (MH⁺ with ⁷⁹Br⁸¹Br, 100%), 517 (MH⁺ with ⁸¹Br₂, 50%). Anal. Calcd for C₁₆H₁₀Br₂N₄O₂S₂.0.1 EtOAc: C, 37.66; H, 2.08; N, 10.71. Found C, 37.64; H, 2.20; N, 10.71.

### Biological Activity of Compounds of the Invention

### A. Inhibition of Isolated Enzyme

Compounds were evaluated for their ability to inhibit the Class I PI 3-kinase enzymes p110δ/p85, p110α/p85 and p110β/p85. Reaction mixtures comprising 0.1 μg of recombinant enzyme, 10 μg of L-α-phosphatidylinositol, the compound (DMSO only or DMSO+compound to a final concentration of 1%), 2× Lipid Kinase Buffer (40 mM Tris-HCl pH 7.4, 200 mM NaCl, 1 mM EDTA) were activated by the addition of an ATP mix (5 mM MgCl₂, 100 μM ATP, 0.1 μL [γ³³P]ATP). Reactions were incubated at room temperature for 1 hour, then stopped by the addition of 1M HCl. The lipids were then extracted using a two step procedure. Firstly, 200 μL of chloroform/methanol (1:1) was added, the biphasic reactions mixed and centrifuged briefly, and the inorganic phase was removed and discarded. Following this 80 μL of methanol:HCl (1:1) was added and the same procedure followed. The organic phase (70 μL) was then transferred to a clean 1.6 mL tube and the reactions were dried using a Speedvac, with no heating, for 30 minutes. The reactions were spotted onto TLC plates (Merck Ltd) and developed for 1 hour in propanol-1:2 M acetic acid (13:7). The TLC plates were then dried at room temperature and quantified using a phosphorimager (StormImager, Amersham). Nine inhibitor concentrations were used to determine the IC₅₀. Each experiment was performed twice and the average IC₅₀ value used.

### B. Cellular Growth Inhibition

The compounds were evaluated against two early passage human cell lines NZB5 and NZOV9, whose development and culture have been described [Marshall et al, Oncol. Res. 2004, 14, 297]. The cells were grown in ITS medium (α-modified minimal essential medium supplemented insulin, transferrin, selenite and 5% fetal bovine serum) and grown on 96-well tissue culture plates under an atmosphere of 5% O₂, 5% CO₂ and 90% N₂. Individual wells contained 500-1000 cells (depending on the growth rate) in a volume of 150 μL. Compounds were added at 10-fold concentration steps to a maximum of 20 μM and plates were incubated for five days, with ³H-thymidine being added over the last 6 h. Cells were harvested and incorporated radioactivity measured. Duplicate samples were analyzed for each compound dose with multiple control samples. Data were fitted by a least-squares method to an exponential of the form y=y₀+ae^{-bx}, where y is the radioactivity (corrected for background and normalized to 100% of the control) x is the radiation dose, and y₀, a and b are variables, and the IC₅₀ value defined as the compound concentration reducing ³H-thymidine levels by 50%.

TABLE 2
Biological data for selected compounds of Table 1.

| | |
|---|---|
| IC₅₀ (nM) | IC₅₀ (μM) |

| No | p110α | p110β | p110δ | NZB5 | NZOV9 |
|---|---|---|---|---|---|
| E1 | 190 | >10000 | 3400 | 0.47 | 0.66 |
| E2 | 110 | >1000 | 640 | 0.05 | 0.07 |
| E4 | 870 | >1000 | >1000 | 15 | 20 |
| E6 | 260 | >1000 | 790 | 1.8 | 1.5 |
| E9 | 230 | >1000 | >1000 | 0.94 | 0.52 |
| E15 | 23 | 720 | 240 | 0.04 | 0.05 |
| E17 | 330 | >1000 | >1000 | 0.74 | 0.67 |
| E18 | 1.4 | 47 | 43 | 0.16 | 0.11 |
| E27 | 86 | 870 | 340 | >20 | 14 |
| E33 | 1.8 | 300 | 40 | 0.19 | 0.26 |
| E34 | 0.5 | 94 | 24 | 0.88 | 0.35 |
| E40 | 3.0 | 23 | 25 | 3.2 | 0.62 |
| E42 | 13 | 620 | 580 | 2.5 | 4.9 |
| E46 | 30 | 200 | 130 | 0.37 | 0.40 |
| E47 | 4.5 | 230 | 10 | 0.51 | 0.55 |
| E58 | 9.0 | >1000 | 26 | 9.4 | >20 |
| E63 | 55 | >1000 | 320 | 0.82 | 0.61 |
| E65 | 9.6 | 580 | 440 | 1.1 | 0.68 |
| E71 | 5.8 | 5600 | 200 | 1.3 | 0.66 |
| E73 | 3.4 | 35 | 210 | 0.11 | 0.31 |
| E74 | 32 | 2900 | 350 | 0.27 | 0.55 |
| E77 | 10 | 360 | 380 | 0.06 | 0.05 |
| E80 | 23 | 5800 | 230 | 7.9 | 3.4 |
| E82 | 21 | 72 | 19 | 1.8 | 6.0 |
| E84 | 41 | 48 | 39 | 1.7 | 1.9 |
| E88 | 250 | >1000 | 1450 | 0.08 | 0.07 |
| E90 | 790 | 5500 | 250 | 0.18 | 0.61 |
| E91 | 84 | >1000 | 120 | 0.73 | 2.8 |

The compounds described in Table 1 are all inhibitors of PI 3-kinase. In particular, they inhibit the PI 3-kinase p110α isoform with IC₅₀<1 μM. Some of these examples have IC₅₀<10 nM. In addition, they show inhibition of cellular growth in two early passage cell lines described above with IC₅₀<20 μM in at least one of the cell lines. Some of these examples have IC₅₀<0.1 μM.

Where in the foregoing description, reference has been made to specific components or integers of the invention having known equivalents then such equivalents are herein incorporated as if individually set forth.

Although this invention has been described by way of example and with reference to possible embodiments thereof, it is to be understood that modifications or improvements may be made thereto without departing from the scope or spirit of the invention as defined in the attached claims.

1. A compound of Formula (I), 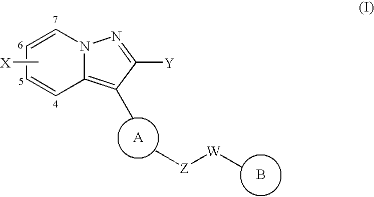
wherein;
X may represent up to two of R, F, Cl, Br, I, OR, OCOR, CONR₂, CO₂R, SO₂R, SO₂NR₂, CN, CF₃, OCF₃, NO₂, NR₂, NHCOR or optionally substituted aryl, placed at any of the available positions 4-, 5-, 6-, 7;
R may be H or C1-C6 saturated or unsaturated alkyl optionally substituted with halogen, OH, OR¹, NHR¹, NR¹₂, or optionally substituted aryl or heteroaryl, or in the case where R forms part of NR₂ this may form an optionally substituted 4-7 membered saturated ring optionally containing one additional heteroatom from the group O, S, NR²;
R¹ is H, C1-C6 saturated or unsaturated alkyl, or optionally substituted aryl or heteroaryl, or in the case when R¹ forms part of NR¹₂ this may form an optionally substituted 4-7 membered saturated ring optionally containing one additional heteroatom from the group O, S, NR²;
R² is H or C1-C6 saturated or unsaturated alkyl;
Y may be H or CH₃;
A represents [symbol] -CH═N—N(R)- [symbol] (where [symbol] is linked to the 3-position of the pyrazole ring of formula I and [symbol] is linked to Z), or any 5-membered heterocylic ring containing up to three of the atoms S, O or N in the ring, and optionally substituted with R, as defined above.
Z represents SOₓ (where x=0-2), CH₂ or CO;
W is absent or (CH₂)_{y} where y=1, 2 or 3;
B is phenyl, naphthyl, or 5- or 6-membered heterocycle or benzoheterocycle, where the heterocylic ring contains up to two of the atoms S, O or N, optionally substituted at any available position with T, which is up to two of F, Cl, Br, I, R, OR, CONR₂, CO₂R, SO₂R, SO₂NHR, CN, CF₃, OCF₃, NO₂, NR₂, NHCOR, where R is defined as above;
or a physiologically acceptable salt or phosphate prodrug or carboxylic acid or amino acid ester prodrug thereof.

2. A compound according to claim 1 wherein X is substituted at the 5-position with R, halogen, OR, OCOR, CONR₂, CO₂R, SO₂R, SO₂NR₂, CN, CF₃, OCF₃, NO₂, NR₂, NHCOR or substituted aryl.

3. A compound according to claim 1 where Z is SO₂ and W is absent.

4. A compound according to claim 1 where B is phenyl, optionally substituted at any position with T.

5. A compound according to claim 1 wherein A is selected from any one of formulae IIa-IIe, where [symbol] is linked to the 3-position of the pyrazole ring of formula (I) and [symbol] is linked to Z:

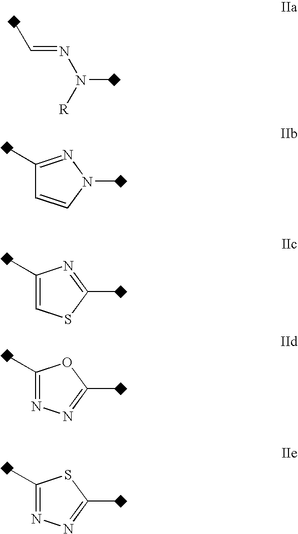

6. A compound as claimed in claim 5 wherein A is formula IIa.

7. A compound according to claim 1 selected from:
N,2-Dimethyl-5-nitro-N′-(pyrazolo[1,5-a]pyridin-3-ylmethylene)benzenesulfonohydrazide
N,2-Dimethyl-N′-((5-methylpyrazolo[1,5-a]pyridin-3-yl)methylene)-5-nitrobenzene-sulfonohydrazide
N,2-Dimethyl-5-nitro-N′-((5-(trifluoromethyl)pyrazolo[1,5-a]pyridin-3-yl)methylene)-benzenesulfonohydrazide
N′-((2,5-Dimethylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N,2-dimethyl-5-nitrobenzene-sulfonohydrazide
Methyl 3-((2-methyl-2-(2-methyl-5-nitrophenylsulfonyl)hydrazono)methyl)pyrazolo-[1,5-a]pyridine-5-carboxylate
3-((2-Methyl-2-(2-methyl-5-nitrophenylsulfonyl)hydrazono)methyl)pyrazolo[1,5-a]-pyridine-5-carboxamide
N′-((5-(2-Hydroxyethyl)pyrazolo[1,5-a]pyridin-3-yl)methylene)-N,2-dimethyl-5-nitro-benzenesulfonohydrazide
N′-((5-Methoxypyrazolo[1,5-a]pyridin-3-yl)methylene)-N,2-dimethyl-5-nitrobenzene-sulfonohydrazide
3-((2-Methyl-2-(2-methyl-5-nitrophenylsulfonyl)hydrazono)methyl)pyrazolo[1,5-a]-pyridin-5-yl acetate
N′-((5-Hydroxypyrazolo[1,5-a]pyridin-3-yl)methylene)-N,2-dimethyl-5-nitrobenzene-sulfonohydrazide
N′-((5-Aminopyrazolo[1,5-a]pyridin-3-yl)methylene)-N,2-dimethyl-5-nitrobenzene-sulfonohydrazide
N′-((5-Chloropyrazolo[1,5-a]pyridin-3-yl)methylene)-N,2-dimethyl-5-nitrobenzene-sulfonohydrazide
N′-((5-Bromopyrazolo[1,5-a]pyridin-3-yl)methylene)-N,2-dimethyl-5-nitrobenzene-sulfonohydrazide
N′-((5-Iodopyrazolo[1,5-a]pyridin-3-yl)methylene)-N,2-dimethyl-5-nitrobenzene-sulfonohydrazide
N,2-Dimethyl-5-nitro-N′-((5-vinylpyrazolo[1,5-a]pyridin-3-yl)methylene)benzene-sulfonohydrazide
N′-((5-Cyclopropylpyrazolo[1,5-a]pyridin-3-yl)methylene)-N,2-dimethyl-5-nitro-benzenesulfonohydrazide
N′-((5-Ethynylpyrazolo[1,5-a]pyridin-3-yl)methylene)-N,2-dimethyl-5-nitrobenzene-sulfonohydrazide
N′-((5-Cyanopyrazolo[1,5-a]pyridin-3-yl)methylene)-N-methyl-3-nitrobenzenesulfonohydrazide
3-Cyano-N′-((5-cyanopyrazolo[1,5-a]pyridin-3-yl)methylene)-N-methylbenzene-sulfonohydrazide
5-Cyano-N′-((5-cyanopyrazolo[1,5-a]pyridin-3-yl)methylene)-N,2-dimethylbenzene-sulfonohydrazide
N′-((5-Cyanopyrazolo[1,5-a]pyridin-3-yl)methylene)-N-methyl-3-(trifluoromethyl)-benzenesulfonohydrazide
N′-((5-Cyanopyrazolo[1,5-a]pyridin-3-yl)methylene)-N,2-dimethyl-5-(trifluoromethyl)-benzenesulfonohydrazide
N′-((5-Cyanopyrazolo[1,5-a]pyridin-3-yl)methylene)-N,2-dimethylbenzenesulfonohydrazide
N′-((5-Cyanopyrazolo[1,5-a]pyridin-3-yl)methylene)-N-methyl-4-nitrobenzenesulfonohydrazide
N′-((5-Cyanopyrazolo[1,5-a]pyridin-3-yl)methylene)-N,2-dimethyl-4-nitrobenzene-sulfonohydrazide
N′-((5-Cyanopyrazolo[1,5-a]pyridin-3-yl)methylene)-5-fluoro-N,2-dimethylbenzene-sulfonohydrazide
5-Bromo-N′-((5-cyanopyrazolo[1,5-a]pyridin-3-yl)methylene)-N,2-dimethyl-benzene-sulfonohydrazide
3-Bromo-N′-((5-cyanopyrazolo[1,5-a]pyridin-3-yl)methylene)-N-methylbenzene-sulfonohydrazide
Methyl 3-(2-((5-cyanopyrazolo[1,5-a]pyridin-3-yl)methylene)-1-methylhydrazinyl-sulfonyl)-4-methylbenzoate
N′-((5-Cyanopyrazolo[1,5-a]pyridin-3-yl)methylene)-N,2-dimethyl-5-(methylsulfonyl)-benzenesulfonohydrazide
N′-((5-Cyanopyrazolo[1,5-a]pyridin-3-yl)methylene)-2-ethyl-N-methyl-5-nitrobenzene-sulfonohydrazide
N′-((5-Cyanopyrazolo[1,5-a]pyridin-3-yl)methylene)-2-isopropyl-N-methyl-5-nitro-benzenesulfonohydrazide
2-Chloro-N′-((5-cyanopyrazolo[1,5-a]pyridin-3-yl)methylene)-N-methyl-5-nitro-benzenesulfonohydrazide
N′-((5-Cyanopyrazolo[1,5-a]pyridin-3-yl)methylene)-2-methoxy-N-methyl-5-nitro-benzenesulfonohydrazide
N′-((5-Cyanopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-5-cyano-N,2-dimethylbenzene-sulfonohydrazide
N′-((5-Cyanopyrazolo[1,5-a]pyridin-3-yl)methylene)-N-(2-hydroxyethyl)-2-methyl-5-nitrobenzenesulfonohydrazide
N′-((5-Bromopyrazolo[1,5-a]pyridin-3-yl)methylene)-N-(2-hydroxyethyl)-2-methyl-5-nitrobenzenesulfonohydrazide
5-Cyano-N′-((5-cyanopyrazolo[1,5-a]pyridin-3-yl)methylene)-N-(2-hydroxyethyl)-2-methylbenzenesulfonohydrazide
N-(2-Hydroxyethyl)-2-methyl-5-nitro-N′-(pyrazolo[1,5-a]pyridin-3-ylmethylene)-benzenesulfonohydrazide
N′-((5-Cyanopyrazolo[1,5-a]pyridin-3-yl)methylene)-2-methyl-5-nitrobenzenesulfonohydrazide
N-Benzyl-N′-((5-cyanopyrazolo[1,5-a]pyridin-3-yl)methylene)-2-methyl-5-nitro-benzenesulfonohydrazide
N′-((5-Cyanopyrazolo[1,5-a]pyridin-3-yl)methylene)-N-ethyl-2-methyl-5-nitrobenzene-sulfonohydrazide
N′-((5-Cyanopyrazolo[1,5-a]pyridin-3-yl)methylene)-N-(2-(diethylamino)ethyl)-2-methyl-5-nitrobenzenesulfonohydrazide
N′-((5-Cyanopyrazolo[1,5-a]pyridin-3-yl)methylene)-N-(2-(dimethylamino)ethyl)-2-methyl-5-nitrobenzenesulfonohydrazide
N′-((5-Cyanopyrazolo[1,5-a]pyridin-3-yl)methylene)-2-methyl-N-(2-morpholinoethyl)-5-nitrobenzenesulfonohydrazide
N′-((5-Cyanopyrazolo[1,5-a]pyridin-3-yl)methylene)-2-methyl-5-nitro-N-(2-(piperidin-1-yl)ethyl)benzenesulfonohydrazide
N′-((5-Cyanopyrazolo[1,5-a]pyridin-3-yl)methylene)-2-methyl-5-nitro-N-(2-(pyrrolidin-1-yl)ethyl)benzenesulfonohydrazide
N′-((5-Bromopyrazolo[1,5-a]pyridin-3-yl)methylene)-2-methyl-5-nitrobenzenesulfonohydrazide
N′-((5-Bromopyrazolo[1,5-a]pyridin-3-yl)methylene)-2-methyl-5-nitro-N-(2-(piperidin-1-yl)ethyl)benzenesulfonohydrazide hydrochloride
N′-((5-Bromopyrazolo[1,5-a]pyridin-3-yl)methylene)-2-methyl-5-nitro-N-(3-(piperidin-1-yl)propyl)benzenesulfonohydrazide hydrochloride
N′-((5-Bromopyrazolo[1,5-a]pyridin-3-yl)methylene)-2-methyl-N-(3-morpholinopropyl)-5-nitrobenzenesulfonohydrazide hydrochloride
2-Methyl-N′-((5-methylpyrazolo[1,5-a]pyridin-3-yl)methylene)-5-nitrobenzenesulfonohydrazide
N′-((5-Cyanopyrazolo[1,5-a]pyridin-3-yl)methylene)-2-fluoro-N-methyl-5-nitro-benzenesulfonohydrazide
N′-((5-Cyanopyrazolo[1,5-a]pyridin-3-yl)methylene)-2-((2-(dimethylamino)ethyl)-(methyl)amino)-N-methyl-5-nitrobenzenesulfonohydrazide
N′-((5-Cyanopyrazolo[1,5-a]pyridin-3-yl)methylene)-2-(2-(dimethylamino)ethylamino)-N-methyl-5-nitrobenzenesulfonohydrazide hydrochloride
N′-((5-Cyanopyrazolo[1,5-a]pyridin-3-yl)methylene)-N-methyl-2-(2-morpholinoethyl-amino)-5-nitrobenzenesulfonohydrazide hydrochloride
N′-((5-Cyanopyrazolo[1,5-a]pyridin-3-yl)methylene)-N-methyl-2-(methyl(2-morpholinoethyl)amino)-5-nitrobenzenesulfonohydrazide hydrochloride
N′-((5-Cyanopyrazolo[1,5-a]pyridin-3-yl)methylene)-N-methyl-2-(methyl(2-(piperidin-1-yl)ethyl)amino)-5-nitrobenzenesulfonohydrazide hydrochloride
N′-((5-Cyanopyrazolo[1,5-a]pyridin-3-yl)methylene)-N-methyl-2-(3-morpholinopropyl-amino)-5-nitrobenzenesulfonohydrazide hydrochloride
N′-((5-Cyanopyrazolo[1,5-a]pyridin-3-yl)methylene)-N-methyl-2-(methyl(2-(methyl-amino)ethyl)amino)-5-nitrobenzenesulfonohydrazide hydrochloride
2-(2-(1H-Imidazol-4-yl)ethylamino)-N′-((5-cyanopyrazolo[1,5-a]pyridin-3-yl)methyl-ene)-N-methyl-5-nitrobenzenesulfonohydrazide hydrochloride
N′-((5-Cyanopyrazolo[1,5-a]pyridin-3-yl)methylene)-N-methyl-5-nitro-2-(pyridin-2-yl-methylamino)benzenesulfonohydrazide hydrochloride
N′-((5-Cyanopyrazolo[1,5-a]pyridin-3-yl)methylene)-N-methyl-5-nitro-2-(pyridin-3-yl-methylamino)benzenesulfonohydrazide hydrochloride
N′-((5-Cyanopyrazolo[1,5-a]pyridin-3-yl)methylene)-N-methyl-5-nitro-2-(pyridin-4-yl-methylamino)benzenesulfonohydrazide hydrochloride
N′-((5-Cyanopyrazolo[1,5-a]pyridin-3-yl)methylene)-N-methyl-2-(methyl(pyridin-3-yl-methyl)amino)-5-nitrobenzenesulfonohydrazide hydrochloride
N′-((5-Cyanopyrazolo[1,5-a]pyridin-3-yl)methylene)-2-(2-(dimethylamino)ethoxy)-N-methyl-5-nitrobenzenesulfonohydrazide hydrochloride
N′-((5-Cyanopyrazolo[1,5-a]pyridin-3-yl)methylene)-N-methyl-2-(2-morpholinoethoxy)-5-nitrobenzenesulfonohydrazide hydrochloride
N′-((5-Cyanopyrazolo[1,5-a]pyridin-3-yl)methylene)-N-methyl-5-nitro-2-(2-(pyrrolidin-1-yl)ethoxy)benzenesulfonohydrazide hydrochloride
N′-((5-Cyanopyrazolo[1,5-a]pyridin-3-yl)methylene)-N-methyl-5-nitro-2-(pyridin-2-yl-methoxy)benzenesulfonohydrazide hydrochloride
N′-((5-Bromopyrazolo[1,5-a]pyridin-3-yl)methylene)-2-fluoro-N-methyl-5-nitro-benzenesulfonohydrazide
N′-((5-Bromopyrazolo[1,5-a]pyridin-3-yl)methylene)-2-(dimethylamino)-N-methyl-5-nitrobenzenesulfonohydrazide
N′-((5-Bromopyrazolo[1,5-a]pyridin-3-yl)methylene)-N-methyl-2-(2-morpholinoethoxy)-5-nitrobenzenesulfonohydrazide hydrochloride
5-Cyano-N′-((5-cyanopyrazolo[1,5-a]pyridin-3-yl)methylene)-2-fluoro-N-methyl-benzenesulfonohydrazide
5-Cyano-N′-((5-cyanopyrazolo[1,5-a]pyridin-3-yl)methylene)-2-(dimethylamino)-N-methylbenzenesulfonohydrazide
5-Cyano-N′-((5-cyanopyrazolo[1,5-a]pyridin-3-yl)methylene)-2-((2-(dimethylamino)-ethyl)(methyl)amino)-N-methylbenzenesulfonohydrazide hydrochloride
5-Cyano-N′-((5-cyanopyrazolo[1,5-a]pyridin-3-yl)methylene)-N-methyl-2-(2-morpholinoethylamino)benzenesulfonohydrazide hydrochloride
5-Cyano-N′-((5-cyanopyrazolo[1,5-a]pyridin-3-yl)methylene)-N-methyl-2-(2-morpholinoethoxy)benzenesulfonohydrazide hydrochloride
2-Chloro-N′-((5-cyanopyrazolo[1,5-a]pyridin-3-yl)methylene)-N-methyl-5-nitropyridine-3-sulfonohydrazide
N′-((5-Cyanopyrazolo[1,5-a]pyridin-3-yl)methylene)-2-((2-(dimethylamino)ethyl)-(methyl)amino)-N-methyl-5-nitropyridine-3-sulfonohydrazide hydrochloride
N′-((5-Cyanopyrazolo[1,5-a]pyridin-3-yl)methylene)-N-methyl-3-nitrobenzohydrazide
N′-((5-Cyanopyrazolo[1,5-a]pyridin-3-yl)methylene)-N,2-dimethyl-5-nitrobenzo-hydrazide
3-((2-Methyl-2-(2-methyl-5-nitrobenzyl)hydrazono)methyl)pyrazolo[1,5-a]pyridine-5-carbonitrile
3-(1-(2-Methyl-5-nitrophenylsulfonyl)-1H-pyrazol-3-yl)pyrazolo[1,5-a]pyridine
3-(1-(3-Nitrophenylsulfonyl)-1H-pyrazol-3-yl)pyrazolo[1,5-a]pyridine
3-(3-(Pyrazolo[1,5-a]pyridin-3-yl)-1H-pyrazol-1-ylsulfonyl)benzonitrile
5-Bromo-3-(1-(2-methyl-5-nitrophenylsulfonyl)-1H-pyrazol-3-yl)pyrazolo[1,5-a]pyridine
4-(5-Bromopyrazolo[1,5-a]pyridin-3-yl)-2-(3-nitrophenylsulfinyl)thiazole
4-(5-Bromopyrazolo[1,5-a]pyridin-3-yl)-2-(3-nitrophenylsulfonyl)thiazole
2-(5-Bromopyrazolo[1,5-a]pyridin-3-yl)-5-(2-methyl-5-nitrophenylthio)-1,3,4-oxa-diazole
2-(5-Bromopyrazolo[1,5-a]pyridin-3-yl)-5-(2-methyl-5-nitrophenylsulfinyl)-1,3,4-oxa-diazole
2-(5-Bromo-2-methylphenylsulfonyl)-5-(5-bromopyrazolo[1,5-a]pyridin-3-yl)-1,3,4-thiadiazole.

8. A compound according to claim 1 in an enantiomeric or diastereomeric form, or mixture of such forms.

9. A compound according to claim 1 including salts thereof, or phosphate or carboxylic acid or amino acid ester prodrugs thereof.

10. A method of cancer prevention or therapy for treating cancers including the step of administering a compound according to claim 1 to a subject in need thereof.

11. The method according to claim 10 further including administering to the subject one or more chemotherapeutic agents and/or therapies.

12. The method according to claim 11 wherein the chemotherapeutic agents are selected from any one or more of:
Alkylation agents (eg cisplatin, carboplatin)
Antimetabolites (eg methotrexate, 5-FU)
Antitumour antibiotics (eg adriamymycin, bleomycin)
Antitumour vegetable alkaloids (eg taxol, etoposide)
Antitumor hormones (eg dexamethasone, tamoxifen)
Antitumour immunological agents (eg. interferon α, β, γ).

13. The method according to claim 11 wherein the therapies are selected from any one or more of:
Radiation therapy or
Surgery.

14. The method according to claim 11 wherein the method includes the step of administering one or more chemotherapeutic agents or therapies to the subject before, during or after the administration of the compound according to claim 1 to the subject.

15. The method of claim 10 wherein the subject is human or other warm blooded animal.

16. A pharmaceutical composition including a compound of claim 1 together with a pharmaceutically acceptable excipient, adjuvant, carrier, buffer or stabiliser.

17. The pharmaceutical composition according to claim 16 in a tablet, capsule, powder, or liquid form.

18. The pharmaceutical composition according to claim 16 wherein the composition is suitable for oral or parenteral administration.

19. The pharmaceutical composition according to claim 16 further including one or more chemotherapeutic agents.

20. A method for the manufacture of a medicament for the treatment of cancer comprising combining a compound according to claim 1 with a pharmaceutically acceptable excipient, adjuvant, carrier, buffer or stabiliser.

21. The method according to claim 20 wherein the medicament is in tablet, capsule, powder or liquid form.

22. The method according to claim 20 wherein the medicament is suitable for oral or parenteral administration.

23. A method of making a compound according to claim 1, the method including the step of modifying a pyrazolo[1,5-a]pyridine-3-carbonyl compound of Formula III 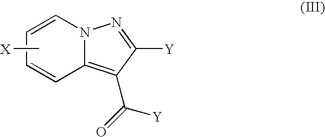
wherein variables X and Y are as defined in claim 1 and V is H, CH₃ or alkoxy.

24. The method according to claim 23 wherein the method proceeds via an intermediate of Formula IV 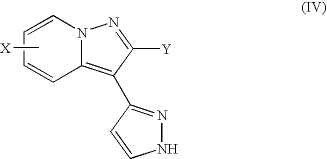
wherein variables X and Y are as defined in claim 1.

25. A method according to claim 23 wherein the compound according to claim 1 is compounds Ia to Id, in which A, R, T, X, Y and Z are as defined in claim 1, 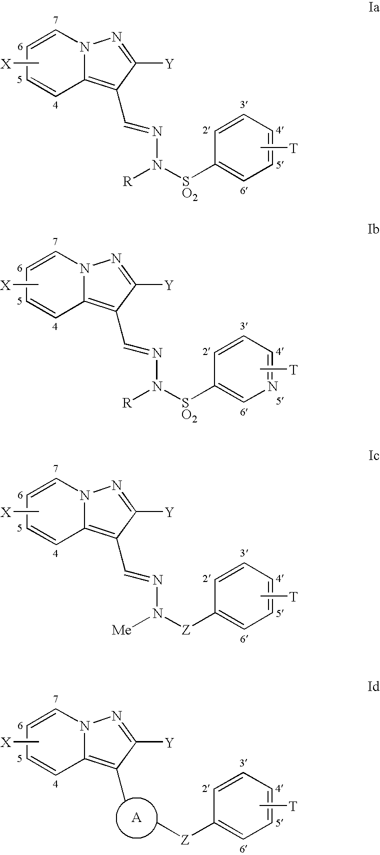
and the method includes:
(i) condensation with a hydrazine followed by sulfonylation for compounds of Formula Ia and Ib;
(ii) condensation with a sulfonohydrazide followed by acylation for compounds of Formula Ia;
(iii) reaction with methylhydrazine sulphate followed by acylation for compounds of Formula Ic where Z=CO;
(iv) reaction with an alkyl hydrazine for compounds of Formula Ic where Z=CH₂;
(v) condensation with methylhydrazine sulphate followed by sulfonylation and then nucleophilic substitution for compounds of Formula Ia and Ib;
(vi) reaction with DMFdma then cyclisation with hydrazine, followed by sulfonylation, for compounds of Formula Id where A is a pyrazole ring;
(vii) bromination then cyclisation with dithiocarbamate, coupling with a boronic acid followed by oxidation for compounds of Formula Id where A is a thiazole ring;
(viii) reaction with hydrazine then cyclisation with CS₂, coupling with a boronic acid followed by oxidation for compounds of Formula Id where A is a 1,3,4-oxadiazole ring; or
(ix) reaction with thiosemicarbazide then cyclisation with H₂SO₄, followed by diazotisation and chlorination, then substitution with a sulfinate salt for compounds of Formula Id where A is a 1,3,4-thiadiazole ring.

26. A compound according to claim 1 obtained by a method according claim 23.

27. A compound according to claim 7 prepared by a method according to claim 23.

28-46. (canceled)
